(19) United States
(12) Patent Application Publication
(10) Pub. No.: US 2026/0258160 A1
(43) Pub. Date: Sep. 3, 2026

(54) NOVEL PREPARATION OF OLIGONUCLEOTIDE CONJUGATES USING TRANSITION METALS OR SALTS THEREOF

(71) Applicant: Avidity Biosciences, Inc., San Diego, CA (US)

(72) Inventors: Joel Daniel ARIAS, San Diego, CA (US); Michael Caramian COCHRAN, Palo Alto, CA (US); Stephanie Elizabeth DESANTI, San Diego, CA (US); Venkata Ramana DOPPALAPUDI, San Diego, CA (US); Son Ngoc LAM, San Diego, CA (US); Noah Robert LUTHER, San Diego, CA (US); Matthew Norman McQUEEN, Poway, CA (US); Arvind BHATTACHARYA, San Diego, CA (US)

(21) Appl. No.: 19/425,930

(22) Filed: Dec. 18, 2025

Related U.S. Application Data
(60) Provisional application No. 63/736,484, filed on Dec. 19, 2024.

(51) Int. Cl. C07K 19/00; C07K 1/107

(52) U.S. Cl. CPC C07K 19/00; C07K 1/1077

(57) ABSTRACT

Disclosed herein is a novel preparation and efficient, large-scale manufacturing methods of oligonucleotide conjugates using transition metals or salts thereof.

### CROSS REFERENCE

This application claims the benefit of U.S. Provisional Application No. 63/736,484 filed Dec. 19, 2024, which is incorporated herein by reference in its entirety.

### SEQUENCE LISTING

The instant application contains a Sequence Listing which has been submitted electronically in XML format and is hereby incorporated by reference in its entirety. Said XML copy, created on May 11, 2026, is named 72904-712_201_SL.xml and is 187,450 bytes in size.

### BACKGROUND OF THE DISCLOSURE

Gene suppression by oligonucleotide-induced gene silencing provides several levels of control: transcription inactivation, small interfering RNA (siRNA)-induced mRNA degradation, and siRNA-induced transcriptional attenuation. In some instances, RNA interference (RNAi) provides long lasting effect over multiple cell divisions. As such, RNAi represents a viable method useful for drug target validation, gene function analysis, pathway analysis, and disease therapeutics. Although the in vivo delivery of RNAi therapeutics in the liver has been successful, the delivery of RNAi agent to other tissues beside liver remains difficult. Recent successful treatments of muscle diseases with RNAi agents conjugated to antibodies delivering RNAi therapeutics to muscle cells demonstrated the breakthrough of targeted delivery of RNAi agent to other tissues beside liver. RNAi agents, such as siRNA, conjugated to antibodies as therapeutics can be used to treat patients with unmet need and require the manufacturing of these therapeutics in large scale. However, current large-scale methods of manufacturing of RNAi agent conjugated to antibodies are costly and inefficient resulting in low yields and heterogenous mixtures of antibody-oligonucleotide conjugates.

Therefore, there is a need for new improved methods for efficient, cost effective, and scalable manufacturing of antibody-oligonucleotide conjugates.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

Provided herein are novel and improved methods of preparing and manufacturing binding moiety-oligonucleotide conjugates. The binding moiety can be, inter alia, an antibody, or fragment thereof. The novel methods of manufacturing binding moiety-oligonucleotide conjugates described herein are efficient, cost effective, and scalable. The methods of preparing these binding moiety-oligonucleotide conjugates comprise the step of conjugating antibodies to oligonucleotide in the presence of one or more transition metals (such as zinc (Zn) or zinc salts). In addition, the methods provide methods for preparing, separating, identifying, and characterizing antibody-oligonucleotide conjugates that include siRNA conjugates or antisense oligonucleotide (ASO) conjugates. In addition, the manufacturing methods provide methods of preparing, conjugating, separating identifying, and characterizing 1 oligonucleotide conjugated to an antibody at a drug to antibody ratio of 1 (DAR1) or 2 oligonucleotides conjugated to an antibody at a drug to antibody ratio of 2 (DAR2). In addition, the manufacturing methods provide methods of preparing, conjugating, separating identifying, and characterizing 1 siRNA conjugated to an antibody at a drug to antibody ratio of 1 (DAR1) or 2 siRNAs conjugated to an antibody at a drug to antibody ratio of 2 (DAR2). In addition, the manufacturing methods provide methods of preparing, conjugating, separating identifying, and characterizing 1 ASO conjugated to, e.g., an antibody (or fragment thereof) at a drug to antibody ratio of 1 (DAR1) or 2 ASOs conjugated to an antibody at a drug to antibody ratio of 2 (DAR2).

In some aspects, disclosed herein is a method of preparing a binding moiety-oligonucleotide conjugate comprising the steps of: a) contacting a binding moiety in an acidic buffer with a reducing agent and a transition metal or its salts thereof to generate a first mixture; b) contacting the first mixture with an oxidizing agent to generate a second mixture; c) adding an oligonucleotide molecule conjugated with a linker to the second mixture to generate a binding moiety-oligonucleotide conjugate comprising the binding moiety conjugated to the oligonucleotide molecule via the linker; and d) isolating the binding moiety-oligonucleotide conjugate, thereby preparing the binding moiety-oligonucleotide conjugate. In some aspects, the acidic buffer has a pH of less than 7.0. In some aspects, the acidic buffer has a pH of about 6.5. In some aspects, the acidic buffer comprises a citrate/sucrose buffer or a histidine buffer. In some aspects, the step a) comprises about 10 molar equivalents of the reducing agent. In some aspects, the reducing agent comprises tris(2-carboxyethyl)phosphine (TCEP) or dithiothreitol (DTT). In some aspects, the step a) comprises about 1-10 molar equivalents of the transition metal or its salts thereof. In some aspects, the transition metal or its salts thereof comprises zinc (Zn) or its salts thereof, silver (Ag) or its salts thereof, or gold (Au) or its salts thereof. In some aspects, the transition metal or its salts thereof comprises zinc citrate, zinc trifluoromethane sulfonate, or zinc acetylacetonate. In some aspects, the step a) further comprises purifying the first mixture using a phosphate buffer, HEPES buffer, a MES buffer. In some aspects, (i) the binding moiety contacts the reducing agent and the transition metal or its salts thereof concurrently, or (ii) the binding moiety contacts the reducing agent prior to contacting the transition metal or its salts thereof. In some aspects, the step b) comprises less than about 15, 14, 13, 12 or 11 molar equivalents of the oxidizing agent. In some aspects, the step b) comprises about 1-10 molar equivalents of the oxidizing agent. In some aspects, the oxidizing agent comprises dehydroascorbic acid (DHAA). In some aspects, the step c) comprises about 1-3 molar equivalents of the oligonucleotide molecule conjugated with the linker (oligonucleotide-linker). In some aspects, the step c) comprises about 1, 1.05, 1.1, 1.5, 2, 2.15, 2.25, 2.5 or 3 molar equivalents of the oligonucleotide-linker. In some aspects, the step d) comprises isolating the binding moiety-oligonucleotide conjugate via chromatography or filtration. In some aspects, the chromatography comprises strong anion chromatography (SAX). In some aspects, the filtration comprises tangential flow filtration (TFF). In some aspects, the method further comprises e) adding an additional oligonucleotide-linker to the isolated binding moiety-oligonucleotide conjugate, wherein the additional oligonucleotide-linker comprises an oligonucleotide molecule that is different from the oligonucleotide molecule in step c), thereby obtaining the binding moiety-oligonucleotide conjugate having different oligonucleotides. In some aspects, the binding moiety is an antibody or antigen binding fragment thereof. In some aspects, the binding moiety comprises a lipid. In some aspects, the lipid comprises a C16, C20, or C22 alkyl chain. In some aspects, the oligonucleotide comprises an siRNA, an ASO, or a PMO. In some aspects, the siRNA comprises a guide strand and a passenger strand, wherein the passenger strand is modified with a lipid moiety. In some aspects, the lipid moiety comprises a C16, C20, or C22 alkyl chain. In some aspects, the lipid moiety is conjugated to 3^{rd} or 6^{th} nucleotide from the 5′ end of the passenger strand. In some aspects, the linker comprises a maleimide group. In some aspects, the linker comprises a MCC linker, a MC linker, a MBS linker, or a bismaleimide (Bismal) linker. In some aspects, a yield of the isolated binding moiety-oligonucleotide conjugate having the DAR of about 1 or about 2 is greater than 50%, 60%, 70%, 80% or more.

Also disclosed herein, in some aspects, is an antibody-Zn complex comprising a zinc and an antibody or antigen binding fragment thereof: wherein the zinc is coordinated with a first histidine residue and a first cysteine residue on a first heavy chain of the antibody or antigen binding fragment thereof, a second histidine residue on a second heavy chain of the antibody or antigen binding fragment thereof, and a second cysteine residue on a first light chain of the antibody or antigen binding fragment thereof. In some aspects, the antibody comprises an IgG1 framework. In some aspects, the antibody is a human IgG1 antibody or a humanized IgG1 antibody.

Also disclosed herein, in some aspects, is a method of preparing an antibody-oligonucleotide conjugate or an antigen binding fragment-oligonucleotide conjugate comprising steps of: a) contacting an antibody or antigen binding fragment thereof with a reducing agent; b) reacting the antibody or antigen binding fragment thereof with a transition metal or its salts thereof; c) contacting the antibody or antigen binding fragment thereof from step b) with an oxidizing agent; d) quenching the oxidizing agent using a mineral or organic buffer; e) chelating the transition metal using a chelating agent; and f) contacting an oligonucleotide molecule conjugated with a linker (oligonucleotide-linker) to the antibody or antigen binding fragment thereof, thereby generating an antibody-oligonucleotide conjugate or antigen binding fragment-oligonucleotide conjugate. In some aspects, the organic or mineral buffer comprises a borate buffer. In some aspects, the step d) is performed for about 1-2 hours and the step e) is performed for about less than 1 hour. In some aspects, the step d) is performed for about 1 hour and the step e) is performed for about 15-30 minutes. In some aspects, the method further comprises purifying the antibody or antigen binding fragment thereof obtained from the step (a). In some aspects, the method further comprises capping the antibody or antigen binding fragment thereof in the antibody-oligonucleotide conjugate or the antigen binding fragment-oligonucleotide conjugate generated from the step f) using an alkylating reagent. In some aspects, the alkylating agent comprises N-ethylmaleimide (NEM). In some aspects, the step g) comprises about 1-1.5 molar equivalents of the alkylating agent. In some aspects, the chelating agent comprises ethylenediaminetetraacetic acid (EDTA). In some aspects, the method further comprises isolating the antibody-oligonucleotide conjugate or antigen binding fragment-oligonucleotide conjugate via chromatography or filtration. In some aspects, the chromatography comprises strong anion chromatography (SAX). In some aspects, the filtration comprises tangential flow filtration (TFF).

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below:

FIGS. 1A-1B illustrate flow charts for the preparation of antibody-oligonucleotide (e.g., siRNA) conjugates without Zn or with Zn. FIG. 1A illustrates a flow chart for the preparation of antibody-oligonucleotide (e.g., siRNA) conjugates without Zn. FIG. 1B illustrates a flow chart for the preparation of antibody-oligonucleotide (e.g., siRNA) conjugates with Zn.

FIG. 2 illustrates a diagram showing the reaction steps for preparation of antibody-siRNA conjugates using Zn.

FIGS. 3A-3B illustrate strong anion exchange (SAX) chromatograms for the DAR distribution of antibody-siRNA conjugate reaction mixture. FIG. 3A illustrates the strong anion exchange chromatogram for the DAR distribution of anti-TfR1 antibody-siRNA conjugate reaction mixture prepared with a method without Zn. FIG. 3B illustrates the strong anion exchange chromatogram for the DAR distribution of anti-TfR1 antibody-siRNA conjugate reaction mixture prepared with Zn.

FIGS. 4A-4D illustrate non-reduced capillary gel electrophoresis chromatograms of DAR1 antibody-siRNA conjugates. FIG. 4A shows a non-reduced capillary gel electrophoresis chromatogram of DAR1 antibody-siRNA conjugates prepared without Zn. FIG. 4B illustrates a diagram showing the location of the siRNA and NEM on the antibody of the antibody-siRNA conjugates prepared without Zn. FIG. 4C shows non-reduced capillary gel electrophoresis chromatograms of DAR1 antibody-siRNA conjugates prepared with Zn. FIG. 4D illustrates a diagram showing the location of the siRNA and NEM on the antibody of the antibody-siRNA conjugates prepared with Zn.

FIGS. 5A-5B illustrate reduced capillary gel electrophoresis chromatograms of DAR1 antibody-siRNA conjugates. FIG. 5A shows a reduced capillary gel electrophoresis chromatogram of DAR1 antibody-siRNA conjugates without Zn. FIG. 5B shows a reduced capillary gel electrophoresis chromatogram of DAR1 antibody-siRNA conjugates with Zn.

FIGS. 6A-6D illustrate mass chromatograms of non-reduced DAR1 antibody-siRNA conjugates. FIG. 6A shows a mass chromatogram of the non-reduced DAR1 antibody-siRNA conjugates prepared without Zn. FIG. 6B shows an intact mass chromatogram of non-reduced DAR1 antibody-siRNA conjugates prepared with Zn. FIG. 6C illustrates a diagram showing the location of N-ethylmaleimide (NEM) capping distribution on the antibody of the antibody-siRNA conjugates prepared without Zn. FIG. 6D illustrates a diagram showing the location of the N-ethylmaleimide (NEM) capping distribution of the antibody-siRNA conjugates prepared with Zn.

FIGS. 7A-7B illustrate mass chromatograms of the light chain (LC) component from reduced DAR1 antibody-siRNA conjugates. FIG. 7A shows a mass chromatogram of the light chain component from reduced DAR1 antibody-siRNA conjugates prepared without Zn.

FIG. 7B shows a mass chromatogram of the light chain component from reduced DAR1 antibody-siRNA conjugates prepared with Zn.

FIGS. 8A-8C illustrate a diagram illustrating the FabALACTICA® enzyme digestion of antibody-siRNA conjugates and size exclusion chromatograms of conjugates digested with FabALACTICA. FIG. 8A shows a diagram illustrating the FabALACTICA enzyme digestion of antibody-siRNA conjugates at one specific site at CH1 domain of the antibody and the purification scheme of the Fab and Fc components. FIG. 8B shows a size exclusion chromatogram of DAR1 antibody-siRNA conjugates prepared without Zn and treated with FabALACTICA. FIG. 8C shows a size exclusion chromatogram of DAR1 antibody-siRNA conjugates prepared with Zn and treated with FabALACTICA.

FIGS. 9A-9B show reduced capillary gel electrophoresis chromatograms of DAR2 antibody-siRNA conjugates. FIG. 9A illustrates a reduced capillary gel electrophoresis chromatogram of DAR2 antibody-siRNA conjugates prepared without Zn. FIG. 9B illustrates a reduced capillary gel electrophoresis chromatogram of DAR2 antibody-siRNA conjugates prepared with Zn.

FIGS. 10A-10B illustrate strong cation exchange chromatograms of DAR1 antibody-siRNA conjugates. FIG. 10A illustrates a strong cation exchange chromatogram of DAR1 antibody-siRNA conjugates prepared without Zn. FIG. 10B illustrates a strong cation exchange chromatogram of DAR1 antibody-siRNA conjugates prepared with Zn.

FIG. 11 illustrates a strong anion exchange chromatogram of an antibody-siRNA conjugate with a bismal linker prepared with Zn.

FIGS. 12A-12B illustrate an intact mass chromatogram of a non-reduced antibody-NEM conjugate and a representation of the location of N-ethylmaleimide (NEM) capping distribution on the antibody. FIG. 12A shows an intact mass chromatogram of a non-reduced antibody-NEM conjugate. FIG. 12B illustrates a diagram showing the location of N-ethylmaleimide (NEM) capping distribution on the antibody.

FIGS. 13A-13B illustrate flow charts for the synthesis of DAR1 antibody-siRNA conjugate with Zn and with or without borate. FIG. 13A illustrates a flow chart for the synthesis of DAR1 antibody-siRNA conjugate with Zn. FIG. 13B illustrates a flow chart for the synthesis of DAR1 antibody-siRNA conjugate with Zn and borate.

FIGS. 14A-14B illustrate strong anion exchange chromatograms of antibody-siRNA conjugate reaction mixture prepared with Zn. FIG. 14A illustrates a strong anion exchange chromatograms of antibody-siRNA conjugate reaction mixture prepared with condition b (see Table 9). FIG. 14B illustrates a strong anion exchange chromatograms of antibody-siRNA conjugate reaction mixture prepared with condition g (see Table 9).

FIG. 15 illustrates DAR distribution of antibody-siRNA conjugates prepared by the Zn conjugation method described herein with varying amounts of siRNAs used in the reaction.

FIG. 16 illustrates DAR distribution of antibody-lipid-siRNA conjugates prepared by the Zn conjugation method described herein with different lipid-siRNAs used in the reaction.

FIG. 17 illustrates DAR distribution of antibody-siRNA conjugates prepared by the Zn conjugation method described herein with various linkers used in the reaction

FIG. 18 shows capillary gel electrophoresis chromatograms of antibody-siRNA conjugates with a bismal linker.

FIG. 19 illustrates DAR distribution of antibody-siRNA conjugates prepared by the Zn conjugation method described herein with borate buffer and EDTA used at various amount and reaction times.

### DETAILED DESCRIPTION OF THE DISCLOSURE

Antibody-oligonucleotide conjugation can be achieved through cysteine-based conjugation methods. Site-specific conjugation refers to the process of attaching one or more drugs, e.g., an oligonucleotide, to a specific location on an antibody. Many antibodies contain four interchain disulfide bonds (two disulfide bonds between heavy chains and two disulfide bonds between heavy and light chains) that can be chemically reduced to expose free thiol groups for further conjugation. This conjugation process generates a mixture of antibody-oligonucleotide and/or drug conjugates.

Provided herein is a method of preparing antibody-oligonucleotide conjugates. Further provided herein is a method of preparing conjugates comprising an antibody fragment and an oligonucleotide. Such antibody fragments may include, inter alia, Fab-oligonucleotide conjugates. Such method employs use of a transition metal, such as zinc, or its salts thereof to enhance the selectivity of antibody-oligonucleotide conjugation and to improve the production yield of the antibody-oligonucleotide conjugates.

### Method of Preparation

Described herein is a method of preparing a binding moiety-oligonucleotide conjugate using a metalloid or transition metal. In some aspects, the method comprises steps of: a) contacting a binding moiety to a reducing agent and a transition metal or its salts thereof to generate a first mixture; b) contacting the first mixture to an oxidizing agent to generate a second mixture; c) adding the second mixture to an oligonucleotide molecule conjugated with a linker to generate a binding moiety-oligonucleotide conjugate comprising the binding moiety conjugated to the oligonucleotide molecule via the linker; and d) isolating the binding moiety-oligonucleotide conjugate, thereby preparing the binding moiety-oligonucleotide conjugate. In some instances, the binding moiety is an antibody or antigen binding fragment thereof.

Described herein, in some aspects, is a method of preparing an antibody-moiety conjugate or an antigen binding fragment-moiety conjugate using a transition metal. In some aspects, the method comprises steps of: a) contacting an antibody or antigen binding fragment thereof to a reducing agent and a transition metal or its salts thereof to generate a first mixture; b) contacting the first mixture to an oxidizing agent to generate a second mixture; c) adding the second mixture to a moiety conjugated with a linker to generate an antibody-moiety conjugate or an antigen binding fragment-moiety conjugate comprising the antibody or antigen binding fragment thereof conjugated to the moiety via the linker; and d) isolating the antibody-moiety conjugate or antigen binding fragment-moiety conjugate, thereby preparing antibody-moiety conjugates or antigen binding fragment-moiety conjugate.

Described herein, in some aspects, is a method of preparing an antibody-oligonucleotide conjugate or antigen binding fragment-oligonucleotide conjugate using a transition metal. In some aspects, the method comprises steps of: a) contacting an antibody or antigen binding fragment thereof to a reducing agent and a transition metal or its salts thereof to generate a first mixture; b) contacting the first mixture to an oxidizing agent to generate a second mixture; c) adding the second mixture to an oligonucleotide molecule conjugated with a linker to generate an antibody-oligonucleotide conjugate antigen binding fragment-oligonucleotide conjugate comprising the antibody or antigen binding fragment thereof conjugated to the oligonucleotide via the linker; and d) isolating the antibody-oligonucleotide conjugate or antigen binding fragment-oligonucleotide conjugate, thereby preparing antibody-oligonucleotide conjugate or antigen binding fragment-oligonucleotide conjugate.

Described herein, in some aspects, is a method of preparing an anti-transferrin receptor 1 (TfR1) monoclonal antibody-oligonucleotide conjugate. In some aspects, the method comprises: a) reacting an anti-TfR1 monoclonal antibody with Zn or its salts thereof to form an anti-TfR1 monoclonal antibody-Zn complex; b) reacting the anti-TfR1 monoclonal antibody-Zn complex with a linker attached to oligonucleotide to form an anti-TfR1 monoclonal antibody-oligonucleotide conjugate, thereby preparing an anti-TfR1 monoclonal antibody-oligonucleotide conjugate.

Described herein, in some aspects, is a method of preparing an anti-transferrin receptor 1 (TfR1) monoclonal antibody-Zn complex. In some aspects, the method comprises: reacting an anti-TfR1 monoclonal antibody with Zn or its salts thereof to form an anti-TfR1 monoclonal antibody-Zn complex, wherein Zn forms a Zn-amino acid complex with cysteine residues and histidine residues of heavy and light chains of the antibody; thereby preparing an anti-TfR1 monoclonal antibody-Zn complex.

Further described herein, in some aspects, is a method of preparing an anti-TfR1 monoclonal antibody-oligonucleotide conjugate. In some aspects, the method comprises steps of: a) contacting an anti-TfR1 monoclonal antibody with a reducing agent; b) purifying the reduced anti-TfR1 monoclonal antibody; c) reacting the purified anti-TfR1 monoclonal antibodies with a transition metal or its salts thereof; d) contacting the anti-TfR1 monoclonal antibody from step c) with an oxidizing agent; e) removing the oxidizing agent from the anti-TfR1 monoclonal antibody of step d); f) chelating the transition metal using a chelating agent; g) contacting an oligonucleotide molecule conjugated with a linker (oligonucleotide-linker) to the anti-TfR1 monoclonal antibody, thereby generating an anti-TfR1 monoclonal antibody-oligonucleotide conjugate; h) capping the anti-TfR1 monoclonal antibody using an alkylating reagent; and i) isolating the antibody-oligonucleotide conjugate, thereby preparing an anti-TfR1 monoclonal antibody-oligonucleotide conjugate.

### Reduction Step

In some aspects, the preparation method described herein comprises a reduction step. In some aspects, the reduction step involves a reducing agent. In some aspects, the reducing agent refers to a reductant that breaks disulfide bonds. Exemplary reducing agents include, but are not limited to, Tris(2-carboxyethyl)phosphine (TCEP), tris(2-carboxyethyl)phosphine hydrochloride (TCEP-HCl), dithiothreitol (DTT), Dithioerythritol (DTE), Glutathione, or beta-mercaptoethanol (BME). In some aspects, the reducing agent is a thiol-free reductant for protein and peptide disulfide bonds. In some aspects, the reducing agent is TCEP or TCEP-HCL. In some aspects, the reducing agent is TCEP. In some aspects, the reducing agent is dithiothreitol (DTT). In some aspects, the amount of reducing agent molar equivalents used during the reduction step controls the drug-to-antibody ratio. In some aspects, the amount of reducing agent ranges from about 0.05 to about 10 molar equivalents relative to the antibody. In some aspects, the amount of reducing agent ranges from about 0.1 to about 10 molar equivalents. In some aspects, the amount of reducing agent ranges from about 0.5 to about 10 molar equivalents. In some aspects, the amount of reducing agent ranges from about 1 to about 10 molar equivalents. In some aspects, the amount of reducing agent ranges from about 2 to about 10 molar equivalents. In some aspects, the amount of reducing agent ranges from about 3 to about 10 molar equivalents. In some aspects, the amount of reducing agent ranges from about 4 to about 10 molar equivalents. In some aspects, the amount of reducing agent ranges from about 5 to about 10 molar equivalents. In some aspects, the amount of reducing agent ranges from about 6 to about 10 molar equivalents. In some aspects, the amount of reducing agent ranges from about 7 to about 10 molar equivalents. In some aspects, the amount of reducing agent ranges from about 8 to about 10 molar equivalents. In some aspects, the amount of reducing agent ranges from about 9 to about 10 molar equivalents. In some aspects, the amount of reducing agent ranges from about 10 to about 100 molar equivalents. In some aspects, the amount of reducing agent ranges from about 10 to about 90 molar equivalents. In some aspects, the amount of reducing agent ranges from about 10 to about 80 molar equivalents. In some aspects, the amount of reducing agent ranges from about 10 to about 70 molar equivalents. In some aspects, the amount of reducing agent ranges from about 10 to about 60 molar equivalents. In some aspects, the amount of reducing agent ranges from about 10 to about 50 molar equivalents. In some aspects, the amount of reducing agent ranges from about 10 to about 40 molar equivalents. In some aspects, the amount of reducing agent ranges from about 10 to about 30 molar equivalents. In some aspects, the amount of reducing agent ranges from about 10 to about 20 molar equivalents. In some aspects, the amount of reducing agent ranges from about 10 to about 15 molar equivalents. In some aspects, the amount of reducing agent ranges from about 5 to about 15 molar equivalents. In some aspects, the amount of reducing agent ranges from about 10 to about 15 molar equivalents. In some aspects, the amount of reducing agent ranges from about 8 to about 12 molar equivalents. In some aspects, the amount of reducing agent is about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more molar equivalents. In some aspects, the amount of reducing agent is at least about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more molar equivalents. In some aspects, the amount of reducing agent is at most about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 molar equivalents. In some aspects, the amount of reducing agent molar equivalents is about 2 molar equivalents. In some aspects, the amount of reducing agent is about 5 molar equivalents. In some aspects, the amount of reducing agent is about 10 molar equivalents. In some aspects, the amount of reducing agent is about 15 molar equivalents. In some aspects, the concentration of reducing agent ranges from about 0.04 mM to about 20 mM. In some aspects, the concentration of reducing agent ranges from about 0.1 mM to about 10 mM. In some aspects, the concentration of reducing agent ranges from about 0.1 mM to about 5 mM. In some aspects, the concentration of reducing agent ranges from about 0.1 mM to about 2.5 mM. In some aspects, the concentration of reducing agent ranges from about 0.1 mM to about 2.0 mM. In some aspects, the concentration of reducing agent ranges from about 0.1 mM to about 1.5 mM.

In some aspects, the reducing agent is in the buffer. In some aspects, the buffer comprises citrate/sucrose buffer, PBS buffer, HEPES buffer, histidine buffer, TBS buffer, MES buffer, acetate buffer, or any suitable buffer. In some aspects, the buffer comprises citrate buffer. In some aspects, the buffer comprises sucrose excipient. In some aspects, the buffer comprises citrate/sucrose buffer. In some aspects, the buffer comprises a histidine buffer.

In some aspects, pH in the reduction step ranges from about 5.5 to about 8. In some aspects, the optimal pH ranges from about 6.5 to about 7.5. In some aspects, pH in the reduction step is less than about 7.0. In some aspects, pH in the reduction step is about 6.5. In some aspects, pH in reduction step is about 6.0. In some aspects, a citrate buffer, a sucrose buffer, a histidine buffer, or any combination thereof is used to maintain an acidic pH (e.g., less than about 7.0) in the reduction step. In some aspects, citrate is used at about 10 mM to about 100 mM at pH about 5.0 to about 6.5 to maintain a mildly acidic pH. In one aspect, the mixture contains 50 mM citrate and 300 mM sucrose at pH 6.5.

In some aspects, the reduction step is performed at a temperature range from about 4° C. to about 37° C. In some aspects, the reduction step is performed at a temperature range from about 4° C. and about 20° C. In some aspects, the reduction step is performed at room temperature. In some aspects, the reduction time is about 0.5, 1, 1.5, 2, 2.5, 3, 4, or 5 hours or more. In some aspects, the reduction time is less than about 0.5, 1, 1.5, 2, 2.5, 3, 4, or 5 hours. In some aspects, the reduction time is more than about 0.5, 1, 1.5, 2, 2.5, 3, 4, or 5 hours. In some aspects, the reduction time is between about 0.5 hour and about 2 hours. In some aspects, the reduction time is between about 1 hour and about 3 hours. In some aspects, the reduction time is between about 1 hour and about 2 hours. In some aspects, the reduction time is between about 1.5 hours and about 2.5 hours. In some aspects, the reduction time is about 1 hour. In some aspects, the reduction time is about 2 hours.

In some instances, after reducing disulfide bonds, filtration is performed to remove the reducing agent and any small molecule byproducts while retaining the antibody molecule. In some aspects, removing the reducing agent from antibody mixture comprises diafiltration. In some aspects, the molecular weight cut-off (MWCO) of the diafiltration membrane is about 10 kDa to about 60 kDa. In one aspect, the molecular weight cut-off (MWCO) of the diafiltration membrane is about 50 kDa. In some aspects, the buffer for diafiltration comprises PBS buffer, phosphate buffer, HEPES buffer, or MES buffer. In some aspects, the buffer for diafiltration is a phosphate buffer. In some aspects, the buffer for diafiltration is a HEPES buffer. In some aspects, the buffer for diafiltration is a MES buffer. In some aspects, the buffer used during diafiltration ranges from about 1 to about 20 diavolumes, about 1 to about 19 diavolumes, about 1 to about 18 diavolumes, about 1 to about 17 diavolumes, about 1 to about 16 diavolumes, about 1 to about 15 diavolumes, about 1 to about 14 diavolumes, about 1 to about 13 diavolumes, about 1 to about 12 diavolumes, about 1 to about 11 diavolumes, or about 1 to about 10 diavolumes. In some aspects, about 1, 2, 3, 4, 5, 6, 7, 8, 9, 10, 11, 12, 13, 14, 15 or more diavolumes of buffer is used during diafiltration. In some aspects, at least about 1, 2, 3, 4, 5, 6, 7, 8, 9, 10, 11, 12, 13, 14, 15, or more diavolumes of buffer is used during diafiltration. In some aspects, about 5 to about 10 diavolumes of buffer is used during diafiltration. In some aspects, the pH of the buffer ranges from about 6 to about 8. In some aspects, the pH of the buffer is about 7.5.

### Metalloid and Transition Metal

In some aspects, the reduction step described herein involves a metalloid or transition metal. In some aspects, the reduction step described herein is performed in the presence of a metalloid. Exemplary metalloids include, but are not limited to arsenic (As) and antimony (Sb). In some aspects, the reduction step described herein is performed in the presence of a transition metal. Exemplary transition metals include, but are not limited to, zinc, cadmium, gold, silver, copper, iron, nickel, cobalt, mercury, lead, and platinum. In some aspects, the transition metal can be present in a salt form in solution to be added to the mixture. In some aspects, the transition metal ion comprises Zn²⁺, Cd²⁺, Hg²⁺, Au³⁺, Ag⁺, Cu²⁺, Fe²⁺, Fe³⁺, Ni²⁺ or Co²⁺. In some aspects, the transition metal ion is Cd²⁺. Exemplary transition metal salts comprising Cd²⁺ include, but not limited to, CdCl₂, CdN(NO₃)₂, CdSO₄, Cd(CH₃COO)₂, CdI₂, CdBr₂, cadmium formate, or cadmium tetrafluoroborate. In some aspects, the transition ion is Hg²⁺. Exemplary transition metal salts comprising Hg²⁺ include, but not limited to, HgCl₂, Hg(NO₃)₂, HgSO₄, Hg(CH₃COO)₂, HgBr₂, mercury(II) formate, or mercury(II) tetrafluoroborate. Exemplary transition metal salts comprising Ag⁺ include, but not limited to, AgCl, AgNO₂, AgSO₄, C₆H₄(OH)COOAg, AgBrO₃. Exemplary transition metal salts comprising Au³⁺ include, but not limited to AuCl₃ or C₄H₆AuNa₂O₄S. In some aspects, the transition metal ion is Zn²⁺. Exemplary transition metal salts comprising Zn²⁺ include, but not limited to, ZnCl₂, ZnN(NO₃)₂, ZnSO₄, Zn(CH₃COO)₂, ZnI₂, ZnBr₂, zinc formate, or zinc tetrafluoroborate. In some aspects, transition metal salts comprises zinc citrate, zinc trifluoromethane sulfonate, or zinc acetylacetonate. In some aspects, the transition metal salt is ZnCl₂. In certain aspects, the transition metal Zn or its salt, ZnCl₂, is added to the mixture to improve the selectivity in disulfide reduction.

In some aspects, the amount of metalloid or transition metal or its salts thereof added to the reaction ranges from about 0.1 to about 20 molar equivalents relative to the antibody. In some aspects, the amount of transition metal or its salts thereof added to the reaction ranges from about 0.1 to about 19 molar equivalents. In some aspects, the amount of transition metal or its salts thereof added to the reaction ranges from about 0.1 to about 18 molar equivalents. In some aspects, the transition metal or its salts thereof added to the reaction ranges from about 0.1 to about 17 molar equivalents. In some aspects, the transition metal or its salts thereof added to the reaction ranges from about 0.1 to about 16 molar equivalents. In some aspects, the transition metal or its salts thereof added to the reaction ranges from about 0.1 to about molar 15 molar equivalents. In some aspects, the transition metal or its salts thereof added to the reaction ranges from about 0.1 to about 14 molar equivalents. In some aspects, the transition metal or its salts thereof added to the reaction ranges from about 0.1 to about molar 13 molar equivalents. In some aspects, the transition metal or its salts thereof added to the reaction ranges from about 0.1 to about 12 molar equivalents. In some aspects, the transition metal or its salts thereof added to the reaction ranges from about 0.1 to about 11 molar equivalents. In some aspects, the transition metal or its salts thereof added to the reaction ranges from about 0.1 to about molar 10 molar equivalents. In some aspects, the transition metal or its salts thereof added to the reaction ranges from about 0.1 to about 9 molar equivalents. In some aspects, the transition metal or its salts thereof added to the reaction ranges from about 0.1 to about 8 molar equivalents. In some aspects, the transition metal or its salts thereof added to the reaction ranges from about 0.1 to about 7 molar equivalents. In some aspects, the transition metal or its salts thereof added to the reaction ranges from about 0.1 to about 6 molar equivalents. In some aspects, the transition metal or its salts thereof added to the reaction ranges from about 0.1 to about 5 molar equivalents. In some aspects, the transition metal or its salts thereof added to the reaction ranges from about 0.1 to about 4 molar equivalents. In some aspects, the transition metal or its salts thereof added to the reaction ranges from about 0.1 to about 3 molar equivalents. In some aspects, the transition metal or its salts thereof added to the reaction ranges from about 0.1 to about 2 molar equivalents. In some aspects, the transition metal or its salts thereof added to the reaction ranges from about 0.5 to about 2 molar equivalents. In some aspects, the transition metal or its salts thereof added to the reaction ranges from about 0.5 to about 1.5 molar equivalents. In some aspects, the transition metal or its salts thereof added to the reaction ranges from about 1 to about 10 molar equivalents. In some aspects, the transition metal or its salts thereof added to the reaction ranges from about 1 to 5 molar equivalents. In some aspects, the transition metal or its salts thereof added to the reaction ranges from about 1 to about 2.5 molar equivalents. In some aspects, the transition metal or its salts thereof added to the reaction ranges from about 1 to 1.5 molar equivalents.

In some aspects, the amount of metalloid or transition metal or its salts thereof added to the reaction is about 0.1, 0.5, 1, 1.1, 1.2, 1.3, 1.4, 1.5, 1.6, 1.7, 1.8, 1.9, 2, 2.5, 3, 4, 5, 6, 7, 8, 9, 10, 11, 12, 13, 14, 15, 16, 17, 18, 19, 20, or more molar equivalents. In some aspects, the amount of transition metal or its salts thereof added to the reaction is at least about 0.1, 0.5, 1, 1.1, 1.2, 1.3, 1.4, 1.5, 1.6, 1.7, 1.8, 1.9, 2, 2.5, 3, 4, 5, 6, 7, 8, 9, 10, 11, 12, 13, 14, 15, 16, 17, 18, 19, 20, or more molar equivalents. In some aspects, the amount of transition metal or its salts thereof added to the reaction is at most about 0.1, 0.5, 1, 1.1, 1.2, 1.3, 1.4, 1.5, 1.6, 1.7, 1.8, 1.9, 2, 2.5, 3, 4, 5, 6, 7, 8, 9, 10, 11, 12, 13, 14, 15, 16, 17, 18, 19, or 20 molar equivalents. In some aspects, the transition metal or its salts thereof added to the reaction is about 1.5 molar equivalents. In some aspects, the transition metal or its salts thereof added to the reaction is 1.5 molar equivalents. In some aspects, the concentration of the transition metal ion ranges from about 10 μM to about 1000 μM. In some aspects, the concentration of the transition metal ion ranges from about 10 μM to about 900 μM. In some aspects, the concentration of the transition metal ion ranges from about 10 μM to about 800 μM. In some aspects, the concentration of the transition metal ion ranges from about 10 μM to about 700 μM. In some aspects, the concentration of the transition metal ion ranges from about 10 μM to about 600 μM. In some aspects, the concentration of the transition metal ion ranges from about 10 μM to about 500 μM. In some aspects, the concentration of the transition metal ion ranges from about 10 μM to about 400 μM. In some aspects, the concentration of the transition metal ion ranges from about 10 μM to about 300 μM. In some aspects, the concentration of the transition metal ion ranges from about 10 μM to about 200 μM. In some aspects, the concentration of the transition metal ion ranges from about 10 μM to about 1000 μM. In some aspects, the concentration of the transition metal ion ranges from about 10 μM to about 500 μM. In some aspects, the concentration of the transition metal ion ranges from about 10 μM to about 250 μM.

In some aspects, the pH for adding a metalloid or transition metal or its salts thereof in the preparation of antibody-oligonucleotide conjugate ranges from about 5.5 to about 8. In some aspects, the pH for adding a transition metal or its salts thereof ranges from about 6 to about 8. In some aspects, the pH for adding a transition metal or its salts thereof ranges from about 6.5 to about 7.5. In some aspects, the transition metal or its salts thereof incubated at a temperature ranges from about 4° C. to about 37° C. In some aspects, the transition metal or its salts thereof incubated at a temperature ranges from about 4° C. and about 20° C. In some aspects, the transition metal or its salts thereof is incubated at room temperature. In some aspects, the transition metal or its salts thereof incubation time is about 0.5 hours, 1 hour, 2 hours, 3 hours, 4 hours, 5 hours, 6 hours, 7 hours, 8 hours, 9 hours, 10 hours, 11 hours, 12 hours or more. In some aspects, the transition metal or its salts thereof incubation time is at least about 0.5 hours, 1 hour, 2 hours, 3 hours, 4 hours, 5 hours, 6 hours, 7 hours, 8 hours, 9 hours, 10 hours, 11 hours, 12 hours or more. In some aspects, the transition metal or its salts thereof incubation time is at most about 0.5 hours, 1 hour, 2 hours, 3 hours, 4 hours, 5 hours, 6 hours, 7 hours, 8 hours, 9 hours, 10 hours, 11 hours, 12 hours, 13 hour, 14 hours, 15 hours, 16 hours, 17 hours, 18 hours, 19 hours, or 20 hours. In some aspects, the transition metal or its salts thereof incubation time is overnight. In some aspects, the transition metal or its salts thereof incubation time is less than 0.5 hours. In some aspects, the transition metal or its salts thereof incubation time is about 0.5 hours. In some aspects, the transition metal or its salts thereof incubation time is between about 0.1 hour and about 0.5 hours. In some aspects, the transition metal or its salts thereof incubation time is between about 0.5 hours and about 1 hour. In some aspects, the transition metal or its salts thereof incubation time is between about 1 hour and about 1.5 hours. In some aspects, the transition metal or its salts thereof incubation time is between about 0.5 hours and about 1.5 hours.

### Oxidation Step

In some aspects, the preparation method described herein comprises an oxidation step. In some aspects, the oxidation step involves an oxidizing agent. In some aspects, the oxidizing agent is utilized for the reformation of the native, inter-chain disulfide bonds in the antibody prior to antibody-oligonucleotide conjugation. Exemplary oxidizing agents include, but not limited to, iodine, hydrogen peroxide (H₂O₂), diamide, copper (II) sulfate (CuSO4), potassium ferricyanide (K₃[Fe₉(CN)₆]) and dehydroascorbic acid (DHAA). In some aspects, the oxidizing agent comprises dehydroascorbic acid (DHAA). In some aspects, the oxidizing agent is dehydroascorbic acid (DHAA).

In some aspects, the amount of the oxidizing agent added to the reaction ranges from about 0.1 to about 20 molar equivalents relative to the antibody. In some aspects, the amount of the oxidizing agent added to the reaction ranges from about 0.5 to about 20 molar equivalents, about 1 to about 20 molar equivalents, about 2 to about 20 molar equivalents, about 3 to about 20 molar equivalents, about 4 to about 20 molar equivalents, about 5 to about 20 molar equivalents, about 6 to about 20 molar equivalents, about 7 to about 20 molar equivalents, about 8 to about 20 molar equivalents, about 9 to about 20 molar equivalents, about 9 to about 15 molar equivalents, about 9 to about 14 molar equivalents, about 9 to about 13 molar equivalents, about 9 to about 12 molar equivalents, or about 9 to about 10 molar equivalents. In some aspects, the amount of the oxidizing agent added to the reaction is less than about 15 molar equivalents, about 14 molar equivalents, about 13 molar equivalents, about 12 molar equivalents, about 11 molar equivalents or about 10 molar equivalents. In some aspects, the amount of the oxidizing agent added to the reaction is about 1-10 molar equivalents.

In some aspects, the oxidizing agent added to the reaction is about 0.5, 1, 2, 3, 4, 5, 6, 7, 8, 9, 10, 11, 12, 13, 14, 15, 16, 17, 18, 19, 20 or more molar equivalents. In some aspects, the oxidizing agent added to the reaction is at least about 0.5, 1, 2, 3, 4, 5, 6, 7, 8, 9, 10, 11, 12, 13, 14, 15, 16, 17, 18, 19, 20 or more molar equivalents. In some aspects, the oxidizing agent added to the reaction is at most about 0.5, 1, 2, 3, 4, 5, 6, 7, 8, 9, 10, 11, 12, 13, 14, 15, 16, 17, 18, 19, 20 molar equivalents. In some aspects, about 10 molar equivalents of oxidizing agent is added to the reaction. In some aspects, about 9 molar equivalents of oxidizing agent is added to the reaction. In some aspects, the concentration of the oxidizing agent ranges from about 0.1 mM to about 20 mM. In some aspects, the concentration of the oxidizing agent ranges from about 0.1 mM to about 10 mM. In some aspects, the concentration of the oxidizing agent ranges from about 0.1 mM to about 5 mM. In some aspects, the concentration of the oxidizing agent ranges from about 0.1 mM to about 2.5 mM.

In some instances, filtration is performed after the oxidation step to remove any oxidizing agents or oxidation by products. In some aspects, removing the oxidizing agent from antibody mixture comprises diafiltration. In some aspects, the molecular weight cut-off (MWCO) of the diafiltration membrane is about 10 kDa to about 60 kDa. In one aspect, the molecular weight cut-off (MWCO) of the diafiltration membrane is about 50 kDa. In some aspects, the buffer for diafiltration comprises PBS buffer, phosphate buffer, or MES buffer. In some aspects, the buffer for diafiltration is phosphate buffer. In some aspects, the buffer used during diafiltration ranges from about 1 to about 20 diavolumes, about 1 to about 19 diavolumes, about 1 to about 18 diavolumes, about 1 to about 17 diavolumes, about 1 to about 16 diavolumes, about 1 to about 15 diavolumes, about 1 to about 14 diavolumes, about 1 to about 13 diavolumes, about 1 to about 12 diavolumes, about 1 to about 11 diavolumes, or about 1 to about 10 diavolumes. In some aspects, about 1, 2, 3, 4, 5, 6, 7, 8, 9, 10, 11, 12, 13, 14, 15 or more diavolumes of buffer is used during diafiltration. In some aspects, at least about 1, 2, 3, 4, 5, 6, 7, 8, 9, 10, 11, 12, 13, 14, 15, or more diavolumes of buffer is used during diafiltration. In some aspects, about 5 to about 10 diavolumes of buffer is used during diafiltration. In some aspects, the pH of the buffer ranges from about 6 to about 8. In some aspects, the pH of the buffer is about 7.5.

In some aspects, the filtration step can be replaced by quenching of the oxidizing agents. This can save 5-24 hours of manufacturing time. In some aspects, the oxidizing agent can be quenched (inactivated) using a mineral or organic buffer. In some aspects, the mineral buffer or the organic buffer used for quenching the oxidizing agent comprises phosphate, borate, HEPES, MOPS, Tris, or citrate buffer. In some aspects, the mineral buffer or the organic buffer used for quenching the oxidizing agent is the borate buffer. In some aspects, sodium tetraborate decahydrate is used in the preparation of the borate buffer. In some aspects, the concentration of the resultant borate buffer ranges from about 10 mM to about 200 mM. In one aspect, the concentration of the borate buffer is about 50 mM. In one aspect, the concentration of the borate buffer is about 10 mM to about 50 mM. In one aspect, the concentration of the borate buffer is about 10 mM. In one aspect, the concentration of the borate buffer is about 20 mM. In one aspect, the concentration of the borate buffer ranges from about 10 mM to about 20 mM. In some aspects, the pH of the borate buffer ranges from about 7 to about 9. In some aspects, the pH of the borate buffer is adjusted to about 7.5. In some aspects, the quenching time is from about 0.5 hours to about 2 hours. In some aspects, the quenching time is from about 0.5 hours to about 1 hour. In some aspects, the quenching time is from about 1 hour to about 2 hours. In some aspects, the quenching time is about 1 hour or about 2 hours.

Metalloid or transition metal or its salts thereof is removed after filtration. In some aspects, a chelating agent is used to remove the transition metal. In some aspects, the chelating agent used for this purpose comprises ethylenediaminetetraacetic acid (EDTA), ethylene glycol tetraacetic acid (EGTA), diethylenetriaminepentaacetic acid (DTPA), ethyl alcohol, polyethylene glycol, propylene glycol, sodium hydroxide, hydrochloric acid, citric acid, tannic acid, or lactic acid. In some aspects, the chelating agent is EDTA. In some aspects, the concentration of the chelating agent in the reaction mixture ranges from about 1 to about 10 mM. In one aspect, the concentration of the chelating agent in the reaction mixture is about 0.5, 1, 2, 3, 4, 5, 6, 7, 8, 9, 10 mM or more. In one aspect, the concentration of the chelating agent in the reaction mixture is at least about 0.5, 1, 2, 3, 4, 5, 6, 7, 8, 9, 10 mM or more. In one aspect, the concentration of the chelating agent in the reaction mixture is at most about 0.5, 1, 2, 3, 4, 5, 6, 7, 8, 9, or 10 mM. In one aspect, the concentration of the chelating agent in the reaction mixture is about 2 mM. In one aspect, the concentration of the chelating agent in the reaction mixture is about 5 mM. In one aspect, the concentration of the chelating agent in the reaction mixture is about 10 mM. In one aspect, the concentration of the chelating agent in the reaction mixture is from about 5 mM to about 10 mM. In one aspect, the concentration of the chelating agent in the reaction mixture is from about 5 mM to about 20 mM.

In some aspects, the quenching step is performed for about 1 to about 2 hours and the chelating step is performed for about 1 hour. In some aspects, the quenching step is performed for about 1 to about 2 hours and the chelating step is performed for less than about 1 hour. In some aspects, the quenching step is performed for about 1 to about 2 hours and the chelating step is performed for 10, 15, 20, 25, 30, 35, 40, 45, 50 or 55 minutes. In some aspects, the quenching step is performed for about 1 hour and the chelating step is performed for about 10-30 minutes. In some aspects, the quenching step is performed for about 2 hours and the chelating step is performed for about 10-30 minutes. In some aspects, the quenching step is performed for about 1 hour and the chelating step is performed for about 15 or 30 minutes. In some aspects, the quenching step is performed for about 2 hours and the chelating step is performed for about 15 or 30 minutes. In some aspects, the quenching step is performed for about 1 hour and the chelating step is performed for about 15 minutes. In some aspects, the quenching step is performed for about 2 hour and the chelating step is performed for about 15 minutes. In some aspects, the quenching step is performed for about 1 hour and the chelating step is performed for about 30 minutes. In some aspects, the quenching step is performed for about 2 hours and the chelating step is performed for about 30 minutes.

### Conjugation Step

In some aspects, the preparation method described herein comprises a conjugation step. In some aspects, the conjugation step involves an addition of an oligonucleotide-linker. As used herein, an “oligonucleotide-linker” refers to a molecule comprising an oligonucleotide molecule coupled to a linker, wherein the linker is suitable to couple to a drug moiety (e.g., oligonucleotide) and/or a binding moiety (e.g., an antibody). In some aspects, the conjugation step involves an addition of an oligonucleotide-linker. In some aspects, the conjugation step involves an addition of an siRNA-linker. In some aspects, the conjugation step involves an addition of an ASO-linker. In some aspects, the conjugation step involves an addition of a PMO-linker. In some aspects, the oligonucleotide is modified with a lipid moiety (e.g., C16, C20, or C22 alkyl chain). In some aspects, the conjugation step involves an addition of a lipid-siRNA-linker. In some aspects, the oligonucleotide-linker (e.g., siRNA-linker, ASO-linker, PMO-linker, lipid-siRNA-linker, etc.) is added to the reaction following the steps comprising reducing step and the oxidizing step. In some aspects, the oligonucleotide-linker added to the reaction ranges from about 0.5 to about 10 molar equivalents. In some aspects, the oligonucleotide-linker added to the reaction ranges from about 0.5 to about 9 molar equivalents. In some aspects, the oligonucleotide-linker added to the reaction ranges from about 0.5 to about 8 molar equivalents. In some aspects, the oligonucleotide-linker added to the reaction ranges from about 0.5 to about 7 molar equivalents. In some aspects, the oligonucleotide-linker added to the reaction ranges from about 0.5 to about 6 molar equivalents. In some aspects, the oligonucleotide-linker added to the reaction ranges from about 0.5 to about 5 molar equivalents. In some aspects, the oligonucleotide-linker added to the reaction ranges from about 0.5 to about 4 molar equivalents. In some aspects, the oligonucleotide-linker added to the reaction ranges from about 0.5 to about 3 molar equivalents. In some aspects, the oligonucleotide-linker added to the reaction ranges from about 0.5 to about 2 molar equivalents. In some aspects, the oligonucleotide-linker added to the reaction ranges from about 0.5 to about 1.5 molar equivalent.

In some aspects, about 0.1, 0.5, 1, 1.5, 2, 2.5, 3, 3.5, 4, 4.5, 5, 5.5, 6, 6.5, 7, 7.5, 8, 8.5, 9, 9.5, 10 or more molar equivalents of the oligonucleotide-linker is added to the reaction. In some aspects, at least about 0.1, 0.5, 1, 1.5, 2, 2.5, 3, 3.5, 4, 4.5, 5, 5.5, 6, 6.5, 7, 7.5, 8, 8.5, 9, 9.5, 10 or more molar equivalents of the oligonucleotide-linker is added to the reaction. In some aspects, at most about 0.1, 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15 molar equivalents of the oligonucleotide-linker is added to the reaction. In some aspects, the oligonucleotide-linker added to the reaction is about 1 molar equivalent. In one aspect, the oligonucleotide-linker added to the reaction is about 1.1 molar equivalent. In some aspects, the oligonucleotide-linker added to the reaction is from about 1 to about 2 molar equivalents. In some aspects, the oligonucleotide-linker added to the reaction is about 1, 1.05, 1.1, 1.2, 1.3, 1.4, or 1.5 molar equivalents. In some aspects, the oligonucleotide-linker added to the reaction is from about 2 to about 4 molar equivalents. In some aspects, the oligonucleotide-linker added to the reaction is from about 2 to about 3 molar equivalents. In some aspects, the oligonucleotide-linker added to the reaction is about 2, 2.15, 2.25, 2.35, 2.45, 2.5, 2.6, 2.7, 2.8, 2.9, or 3 molar equivalents. In some aspects, the oligonucleotide-linker added to the reaction is from about 2.0 to about 2.5 molar equivalents. In some aspects, the oligonucleotide-linker added to the reaction is from about 2.5 to about 3 molar equivalents. In some embodiments, about 1 to about 2 molar equivalents of the oligonucleotide-linker are added to obtain DAR1 antibody-oligonucleotide conjugates. In some embodiments, about 2 to about 3 molar equivalents of the oligonucleotide-linker are added to obtain DAR2 antibody-oligonucleotide conjugates.

In some aspects, two or more oligonucleotide-linkers are added to the reaction. In some embodiments, the two or more oligonucleotide-linkers are same. In some embodiments, the two or more oligonucleotide-linkers are different. In some embodiments, the two or more oligonucleotide-linkers comprises the same oligonucleotide but conjugated to different linkers. In some embodiments, the two or more oligonucleotide-linkers comprises different oligonucleotides conjugated with the same linker.

In some aspects, the concentration of the oligonucleotide-linker ranges from about 0.1 mM to about 1 mM.

In some aspects, the conjugation step further comprise a step of capping the antibody using a capping agent. Exemplary capping agents include, but are not limited to, N-Ethylmaleimide (NEM), iodoacetamide, methyl methanethiosulfonate (MMTS), dimethylmaleic anhydride (DMA), acetic anhydride, and 2-Iminothiolane. In some aspects, the capping agent comprises an alkylating reagent. In some aspects, the alkylating reagent comprises iodoacetamide, N-Ethylmaleimide (NEM), chloroacetamide, bromoacetamide, ethyl methanethiosulfonate (EMTS), 2-Iodoacetamidobenzyl (IAB), vinyl sulfone derivatives, or dimethylsulfate. In some aspects, the alkylating agent comprises NEM.

In some aspects, the alkylating agent added to the reaction ranges from about 0.1 molar equivalent to about 20 molar equivalents. In some aspects, the alkylating agent added to the reaction ranges from about 0.1 molar equivalent to about 19 molar equivalents. In some aspects, the alkylating agent added to the reaction ranges from about 0.1 molar equivalent to about 18 molar equivalents. In some aspects, the alkylating agent added to the reaction ranges from about 0.1 molar equivalent to about 17 molar equivalents. In some aspects, the alkylating agent added to the reaction ranges from about 0.1 molar equivalent to about 16 molar equivalents. In some aspects, the alkylating agent added to the reaction ranges from about 0.1 molar equivalent to about 15 molar equivalents. In some aspects, the alkylating agent added to the reaction ranges from about 0.1 molar equivalent to about 14 molar equivalents. In some aspects, the alkylating agent added to the reaction ranges from about 0.1 molar equivalent to about 13 molar equivalents. In some aspects, the alkylating agent added to the reaction ranges from about 0.1 molar equivalent to about 12 molar equivalents. In some aspects, the alkylating agent added to the reaction ranges from about 0.1 molar equivalent to about 11 molar equivalents. In some aspects, the alkylating agent added to the reaction ranges from about 0.1 molar equivalent to about 10 molar equivalents. In some aspects, the alkylating agent added to the reaction ranges from about 1 molar equivalent to about 10 molar equivalents. In some aspects, the alkylating agent added to the reaction ranges from about 5 molar equivalent to about 10 molar equivalents. In some aspects, the alkylating agent added to the reaction ranges from about 1 molar equivalent to about 3 molar equivalents. In some aspects, the alkylating agent added to the reaction ranges from about 1 molar equivalent to about 2 molar equivalents. In some aspects, the alkylating agent added to the reaction ranges from about 1 molar equivalent to about 1.5 molar equivalents.

In some aspects, about 0.1, 0.5, 1, 2, 3, 4, 5, 6, 7, 8, 9, 10, 11, 12, 13, 14, 15, 16, 17, 18, 19, 20 molar equivalents or more alkylating agent is added to the reaction mixture. In some aspects, at least about 0.1, 0.5, 1, 2, 3, 4, 5, 6, 7, 8, 9, 10, 11, 12, 13, 14, 15, 16, 17, 18, 19, 20 molar equivalents or more alkylating agent is added to the reaction mixture. In some aspects, at most about 0.1, 0.5, 1, 2, 3, 4, 5, 6, 7, 8, 9, 10, 11, 12, 13, 14, 15, 16, 17, 18, 19, or 20 molar equivalents alkylating agent is added to the reaction mixture. In some aspect, about 10 molar equivalents alkylating agent is added to the reaction. In some aspect, about 5 molar equivalents alkylating agent is added to the reaction.

In some aspects, the method of preparing antibody-oligonucleotide conjugates is achieved on random sites of the antibody. In some aspects, the method of preparing antibody-oligonucleotide conjugates is achieved on one or more specific sites. In some aspects, antibody comprising free cysteine residues is capable of conjugation. In some aspects, the antibody comprises 1, 2, 3, 4, 5, 6, 7, 8, or more reactive cysteine residue sites. In some aspects, the antibody comprises one or more conjugation sites at the CH1, hinge region, or CH2 region of the heavy chain. In some aspects, the antibody comprises one or more conjugation sites at the CL region of the light chain.

After zinc coordinates with cysteine and histidine residues (forming a zinc finger motif) located in the heavy chain and/or light chain in the antibody to form an antibody-zinc complex (or Zn-amino acid complex), followed by the steps of oxidation and zinc removal, the oligonucleotide-linker can attach to free cysteine residues that become available once the zinc is removed. In some aspects, the cysteines previously bound to zinc are now free and retain reactive thiol groups. In some aspects, the additional cysteines outside of the zinc finger motif are free and retain reactive thiol groups. In some aspects, the one or more cysteine residues on the heavy chains or light chains of the antibody are free to react with the linker attached to the oligonucleotide (e.g., siRNA, ASO, etc.). In some aspects, one cysteine residue previously bound to zinc on the light chain of the antibody and one cysteine residue previously bound to zinc on the heavy chain of the antibody are free to react with the linker attached to the oligonucleotide. In some aspects, the cysteine residue previously bound to zinc on the light chain of the antibody is free to react with the linker attached to the oligonucleotide. In some aspects, the cysteine residue previously bound to zinc on the heavy chain of the antibody is free to react with the linker attached to the oligonucleotide.

In some aspects, the cysteine residue on the CH1 domain or hinge region of the heavy chain of an antibody is free to react with the linker attached to the oligonucleotide. In some aspects, the cysteine residue on the CL domain or of the light chain of an antibody is free to react with the linker attached to the oligonucleotide. In some aspects, the antibody is any antibody or antigen binding fragment thereof. In some embodiments, the antibody is a monoclonal antibody. In some embodiments, the antibody is an anti-transferrin receptor antibody or antigen binding fragment thereof, an anti-HER2 antibody or antigen binding fragment thereof, or an anti-RSV antibody or antigen binding fragment thereof.

In some aspects, the cysteine residues on the light chain and on the heavy chain of an antibody are free to react with the linker attached to the oligonucleotide (e.g., siRNA, ASO, etc.). In some aspects, the cysteine residues on the light chain and on the heavy chain of an anti-TfR1 antibody are free to react with the linker attached to the oligonucleotide. In some aspects, the cysteine residue on the heavy chain of anti-TfR1 monoclonal antibody is free to react with the linker attached to the oligonucleotide. In some aspects, the cysteine residue on the light chain of anti-TfR1 monoclonal antibody is free to react with the linker attached to the oligonucleotide. In some aspects, the cysteine residue on the CH1 domain or hinge region of the heavy chain of anti-TfR1 monoclonal antibody is free to react with the linker attached to the oligonucleotide. In some aspects, the cysteine residue on the CL domain or of the light chain of anti-TfR1 monoclonal antibody is free to react with the linker attached to the oligonucleotide. In some embodiments, the antibody is an anti-TfR1 monoclonal antibody, wherein the light chain comprises an amino acid sequence selected from SEQ ID NOs: 327-330 and the first and second heavy chain comprise an amino acid sequence selected from SEQ ID NOs: 303-326.

In some aspects, the oligonucleotide-linker is conjugated to one or more cysteine residues of the antibody. In some aspects, the oligonucleotide-linker is conjugated to the cysteine residue on heavy chain of the antibody.

### Isolation Step

In some aspects, the preparation method described herein comprises an isolation step. In some aspects, the antibody-moiety conjugates are isolated and purified after the conjugation. In some aspects, the antibody-oligonucleotide conjugates are isolated and purified after the conjugation. In some aspects, the DAR1 antibody-oligonucleotide conjugate is isolated and purified after the conjugation. In some aspects, the DAR2 antibody-oligonucleotide conjugate is isolated and purified after the conjugation. For example, the chromatography is used for isolating antibody-oligonucleotide conjugates. In some aspects, the chromatography comprises size exclusion chromatography (SEC), affinity chromatography, anion exchange chromatography (IEX), dialysis, ultrafiltration, density gradient, or a combination thereof. In some aspects, the purification step comprises strong anion exchange chromatography SAX. In some aspects, isolating antibody-oligonucleotide conjugates comprise filtration. In some aspects, the filtration comprises tangential flow filtration (TFF).

In some aspects, the isolated antibody-oligonucleotide conjugate has at least one oligonucleotide (e.g., siRNA, ASO, PMO, lipid-siRNA, etc.) attached to the cysteine residues on the heavy and on the light chain of the antibody (e.g., anti-TfR1 monoclonal antibody or antigen binding fragment thereof). In some aspects, the isolated antibody-oligonucleotide conjugate has at least one oligonucleotide (e.g., siRNA, ASO, etc.) attached to the one or more cysteine residues on the CH1 domain of the heavy and one or more cysteine residues on the CL domain of the light chain of the antibody (e.g., anti-TfR1 monoclonal antibody or antigen binding fragment thereof, anti-HER2 antibody or antigen binding fragment thereof, anti-RSV antibody or antigen binding fragment thereof).

In some aspects, the method described herein improves the yield of antibody-oligonucleotide conjugates. In some aspects, the method described herein using zinc improves the yield (i.e., manufacturing yield) of antibody-oligonucleotide conjugates compared to the methods without using zinc or its salts thereof. In some aspects, the method provides antibody-oligonucleotide conjugates with a yield of 50%, 55%, 60%, 65%, 70%, 75%, 80%, 85%, 90%, 95% or more than 95%. In some aspects, the method provides anti-TfR1 antibody-oligonucleotide conjugates with a yield of 50%, 55%, 60%, 65%, 70%, 75%, 80%, 85%, 90%, 95% or more than 95%. In some aspects, the method provides DAR1 antibody-oligonucleotide conjugates with a yield of 50%, 55%, 60%, 65%, 70%, 75%, 80%, 85%, 90%, 95% or more than 95%. In some aspects, the method provides DAR1 anti-TfR1 antibody-oligonucleotide conjugates with a yield of 50%, 55%, 60%, 65%, 70%, 75%, 80%, 85%, 90%, 95% or more than 95%.

In some aspects, the method provides DAR2 antibody-oligonucleotide conjugates with a yield of 50%, 55%, 60%, 65%, 70%, 75%, 80%, 85%, 90%, 95% or more than 95%. In some aspects, the method provides DAR2 anti-TfR1 antibody-oligonucleotide conjugates with a yield of 50%, 55%, 60%, 65%, 70%, 75%, 80%, 85%, 90%, 95% or more than 95%.

In some aspects, the method of preparing antibody-oligonucleotide conjugate in the presence of a transition metal or its salts thereof has an increased yield compared to the method in the absence of a transition metal or its salts thereof. In some aspects, the increase in yield is at least about 15%, 20%, 25%, 30%, 35%, 40%, 45%, 50%, 55%, 60%, 65%, 70%, 75%, 80%, 85%, 90%, 95% or more than 95%.

In some aspects, the method of preparing anti-TfR1 antibody-oligonucleotide conjugate in the presence of a transition metal or its salts thereof has an increased yield compared to the method in the absence of a transition metal or its salts thereof. In some aspects, the increase in yield is at least about 15%, 20%, 25%, 30%, 35%, 40%, 45%, 50%, 55%, 60%, 65%, 70%, 75%, 80%, 85%, 90%, 95% or more than 95%.

In some aspects, the method of preparing DAR1 antibody-oligonucleotide conjugate in the presence of a transition metal or its salts thereof has an increased yield compared to the method in the absence of a transition metal or its salts thereof. In some aspects, the method of preparing DAR1 antibody-oligonucleotide conjugate in the presence of a transition metal or its salts thereof has a yield greater than 40%, 50%, 60%, 70%, 80%, 90%, 95%, or more than 95%. In some aspects, the method of preparing DAR1 antibody-oligonucleotide conjugate in the presence of a zinc or its salts thereof has a yield greater than 40%, 50%, 60%, 70%, 80%, 90%, 95%, or more than 95%. In one aspect, the method of preparing DAR1 antibody-oligonucleotide conjugate in the presence of Zinc or its salts thereof has a yield greater than 60%. In one aspect, the method of preparing the DAR1 antibody-oligonucleotide conjugate in the presence of Zinc or its salts thereof has a yield greater than 70%. In one aspect, the method of preparing the DAR1 antibody-oligonucleotide conjugate in the presence of Zinc or its salts thereof has a yield greater than 80%.

In some aspects, the method of preparing DAR2 antibody-oligonucleotide conjugate in the presence of a transition metal or its salts thereof has an increased yield compared to the method in the absence of a transition metal or its salts thereof. In some aspects, the method of preparing DAR2 antibody-oligonucleotide conjugate in the presence of a transition metal or its salts thereof has a yield greater than 40%, 50%, 60%, 70%, 80%, 90%, 95%, or more than 95%. In some aspects, the method of preparing DAR2 antibody-oligonucleotide conjugate in the presence of a zinc or its salts thereof has a yield greater than 40%, 50%, 60%, 70%, 80%, 90%, 95%, or more than 95%. In one aspect, the method of preparing DAR2 antibody-oligonucleotide conjugate in the presence of Zinc or its salts thereof has a yield greater than 60%. In one aspect, the method of preparing the DAR2 antibody-oligonucleotide conjugate in the presence of Zinc or its salts thereof has a yield greater than 70%. In one aspect, the method of preparing the DAR2 antibody-oligonucleotide conjugate in the presence of Zinc or its salts thereof has a yield greater than 80%.

In some aspects, the method of preparing a DAR1 anti-TfR1 antibody-oligonucleotide conjugate in the presence of a transition metal or its salts thereof has an increased yield compared to the method in the absence of a transition metal or its salts thereof. In some aspects, the method of preparing DAR1 anti-TfR1 antibody-oligonucleotide conjugate in the presence of a transition metal or its salts thereof has a yield greater than 40%, 50%, 60%, 70%, 80%, 90%, 95%, or more than 95%. In some aspects, the method of preparing DAR1 anti-TfR1 antibody-oligonucleotide conjugate in the presence of a zinc or its salts thereof has a yield greater than 40%, 50%, 60%, 70%, 80%, 90%, 95%, or more than 95%. In one aspect, the method of preparing DAR1 anti-TfR1 antibody-oligonucleotide conjugate in the presence of zinc or its salts thereof has a yield greater than 50%. In one aspect, the method of preparing the DAR1 anti-TfR1 antibody-oligonucleotide conjugate in the presence of a zinc or its salts thereof has a yield greater than 70%. In one aspect, the method of preparing the DAR1 anti-TfR1 antibody-oligonucleotide conjugate in the presence of a zinc or its salts thereof has a yield greater than 80%.

In some aspects, the method of preparing a DAR2 anti-TfR1 antibody-oligonucleotide conjugate in the presence of a transition metal or its salts thereof has an increased yield compared to the method in the absence of a transition metal or its salts thereof. In some aspects, the method of preparing DAR2 anti-TfR1 antibody-oligonucleotide conjugate in the presence of a transition metal or its salts thereof has a yield greater than 40%, 50%, 60%, 70%, 80%, 90%, 95%, or more than 95%. In some aspects, the method of preparing DAR2 anti-TfR1 antibody-oligonucleotide conjugate in the presence of a zinc or its salts thereof has a yield greater than 40%, 50%, 60%, 70%, 80%, 90%, 95%, or more than 95%. In one aspect, the method of preparing DAR2 anti-TfR1 antibody-oligonucleotide conjugate in the presence of zinc or its salts thereof has a yield greater than 50%. In one aspect, the method of preparing the DAR2 anti-TfR1 antibody-oligonucleotide conjugate in the presence of a zinc or its salts thereof has a yield greater than 70%. In one aspect, the method of preparing the DAR2 anti-TfR1 antibody-oligonucleotide conjugate in the presence of a zinc or its salts thereof has a yield greater than 80%.

In some aspects, the method described herein provides a final conjugate product (e.g., antibody-drug conjugate, antibody-oligonucleotide conjugate, etc.) with fewer open disulfides. In some aspects, antibody-oligonucleotide conjugate prepared by the method in the presence of a metalloid or transition metal (e.g., zinc) or its salts thereof has fewer open disulfides compared to antibody-oligonucleotide conjugate prepared by the method in the absence of a transition metal or its salts thereof. In some aspects, antibody-oligonucleotide conjugate prepared by the method in the presence of zinc or its salts thereof has fewer open disulfides compared to antibody-oligonucleotide conjugate prepared by the method in the absence of zinc or its salts thereof. In some aspects, anti-HER2 antibody-oligonucleotide conjugate prepared by the method in the presence of a transition metal or its salts thereof as described herein has fewer open disulfides compared to anti-HER2 antibody-oligonucleotide conjugate prepared by the method in the absence of a transition metal or its salts thereof as described herein. In some aspects, anti-HER2 antibody-oligonucleotide conjugate prepared by the method in the presence of zinc or its salts thereof as described herein has fewer open disulfides compared to anti-HER2 antibody-oligonucleotide conjugate prepared by the method in the absence of zinc or its salts thereof. In some aspects, anti-RSV antibody-oligonucleotide conjugate prepared by the method in the presence of a transition metal or its salts thereof as described herein has fewer open disulfides compared to anti-RSV antibody-oligonucleotide conjugate prepared by the method in the absence of a transition metal or its salts thereof. In some aspects, anti-RSV antibody-oligonucleotide conjugate prepared by the method in the presence of zinc or its salts thereof has fewer open disulfides compared to anti-RSV antibody-oligonucleotide conjugate prepared by the method in the absence of zinc or its salts thereof as described herein. In some aspects, anti-TfR1 antibody-oligonucleotide conjugate prepared by the method in the presence of a transition metal or its salts thereof has fewer open disulfides compared to anti-TfR1 antibody-oligonucleotide conjugate prepared by the method in the absence of a transition metal or its salts thereof. In some aspects, anti-TfR1 antibody-oligonucleotide conjugate prepared by the method in the presence of zinc or its salts thereof has fewer open disulfides compared to anti-TfR1 antibody-oligonucleotide conjugate prepared by the method in the absence of zinc or its salts thereof. In some aspects, DAR1 antibody-oligonucleotide conjugate prepared by the method in the presence of a transition metal or its salts thereof has fewer open disulfides compared to DAR1 antibody-oligonucleotide conjugate prepared by the method in the absence of a transition metal or its salts thereof. In some aspects, DAR1 antibody-oligonucleotide conjugate prepared by the method in the presence of zinc or its salts thereof has fewer open disulfides compared to DAR1 antibody-oligonucleotide conjugate prepared by the method in the absence of zinc or its salts thereof. In some aspects, DAR1 anti-TfR1 antibody-oligonucleotide conjugate prepared by the method in the presence of a transition metal or its salts thereof has fewer open disulfides compared to DAR1 anti-TfR1 antibody-oligonucleotide conjugate prepared by the method in the absence of a transition metal or its salts thereof. In some aspects, DAR1 anti-TfR1 antibody-oligonucleotide conjugate prepared by the method in the presence of zinc or its salts thereof has fewer open disulfides compared to DAR1 anti-TfR1 antibody-oligonucleotide conjugate prepared by the method in the absence of zinc or its salts thereof. In some aspects, DAR2 antibody-oligonucleotide conjugate prepared by the method in the presence of a transition metal or its salts thereof has fewer open disulfides compared to DAR2 antibody-oligonucleotide conjugate prepared by the method in the absence of a transition metal or its salts thereof as described herein. In some aspects, DAR2 antibody-oligonucleotide conjugate prepared by the method in the presence of zinc or its salts thereof has fewer open disulfides compared to DAR2 antibody-oligonucleotide conjugate prepared by the method in the absence of zinc or its salts thereof as described herein. In some aspects, DAR2 anti-TfR1 antibody-oligonucleotide conjugate prepared by the method in the presence of a transition metal or its salts thereof has fewer open disulfides compared to DAR2 anti-TfR1 antibody-oligonucleotide conjugate prepared by the method in the absence of a transition metal or its salts thereof as described herein. In some aspects, DAR2 anti-TfR1 antibody-oligonucleotide conjugate prepared by the method in the presence of zinc or its salts thereof as described herein has fewer open disulfides compared to DAR2 anti-TfR1 antibody-oligonucleotide conjugate prepared by the method in the absence of zinc or its salts thereof as described herein.

### Transition Metal Complex

Further described herein, is a method of preparing a binding moiety-transition metal complex. In some aspects, the method comprises reacting a binding moiety with a transition metal (e.g., Zn or its salts) to form a binding moiety-transition metal complex. In some instances, the binding moiety is an antibody or antigen binding fragment thereof. In some instances, the binding moiety is a monoclonal antibody. In some instances, the binding moiety is a full-length antibody. In some instances, the antibody comprises two heavy chains and two light chains. In some instances, the antibody comprises an IgG1 framework.

In some aspects, Zn forms a Zn finger domain with cysteine and histidine residues of the heavy and light chains of the antibody; thereby forming an anti-TfR1 monoclonal antibody-Zn complex.

Further described herein, is a method of preparing an antibody-Zn complex. In some aspects, the method comprises reacting an anti-human TfR1 monoclonal antibody with Zn to form an anti-TfR1 monoclonal antibody-Zn complex. In some aspects, Zn forms a Zn finger domain with cysteine and histidine residues of the heavy and light chains of the antibody; thereby forming an anti-TfR1 monoclonal antibody-Zn complex. In some aspects, the method comprises reacting an anti-HER2 monoclonal antibody with Zn to form an anti-HER2 antibody-Zn complex. In some aspects, Zn forms a Zn finger domain with cysteine and histidine residues of the heavy and light chains of the antibody, thereby forming an anti-HER2 antibody-Zn complex. In some aspects, the method comprises reacting an anti-RSV antibody with Zn to form an anti-RSV antibody-Zn complex. In some aspects, Zn forms a Zn finger domain with cysteine and histidine residues of the heavy and light chains of the antibody, thereby forming an anti-RSV antibody-Zn complex.

In some aspects, the zinc coordinates with 4 or 6 ligands. In some aspects, the zinc coordinates with 6 amino acid residues and forms an octahedral coordination. In some aspects, the zinc coordinates with amino acid residues comprising cysteine (Cys), histidine (His), aspartate (Asp), or glutamate (Glu). In some aspects, the zinc coordinates with four residues comprising cysteine residues, histidine residues or a combination thereof. In some aspects, the zinc coordinates with 4 Cys residues (Cys4). In some aspects, the zinc coordinates with 4 His residues (His4). In some aspects, the zinc coordinates with four residues of cysteine and histidine involving 3 Cys and 1 His residues (Cys3His). In some aspects, the zinc coordinates with four residues of cysteine and histidine involving 2 Cys and 2 His residues (Cys2His2). In some aspects, the Zn finger domain is coordinated with one cysteine on the heavy chain, one cysteine on the light chain of the antibody, and 2 histidine residues on the 2 heavy chains. In some aspects, the Zn finger domain is not coordinated with 2 cysteine and 2 histidine residues on the 2 heavy chains.

In some aspects, the Zn finger domain is formed with Zn coordinated with the cysteine and histidine residues on the antibody. In some aspects, the Zn finger domain is formed with Zn coordinated with 2 cysteine and 2 histidine residues on the antibody. In some aspects, the Zn finger domain is formed with Zn coordinated with a cysteine residue on a heavy chain of the antibody.

In some aspects, the Zn finger domain is formed with Zn coordinated with the cysteine and histidine residues on the anti-TfR1 monoclonal antibody. In some aspects, the Zn finger domain is formed with Zn coordinated with 2 cysteine and 2 histidine residues on the anti-TfR1 monoclonal antibody.

In some aspects, the Zn finger domain is formed with Zn coordinated with a histidine residue and a cysteine residue on a first heavy chain of the antibody, and a histidine residue and a cysteine residue on a second heavy chain of the antibody.

In some aspects, the Zn finger domain is formed with Zn coordinated with a histidine residue and a cysteine residue on a first heavy chain of the antibody, a histidine residue on the second heavy chain of the antibody, and a cysteine residue on a light chain of the antibody. In some aspects, the Zn finger domain is formed with Zn coordinated with a histidine residue and a cysteine residue at the CH1 domain of the first heavy chain of the antibody, a histidine residue at the CH1 domain of the second heavy chain of the antibody, and a cysteine residue at the CL domain of a light chain of the antibody

In some aspects, Zn is coordinated with the cysteine residue and histidine residue on a first heavy chain comprising HCDR1 having an amino acid sequence of SEQ ID NO: 281, HCDR2 having an amino acid sequence selected from SEQ ID NOs: 282, 284 or 285, and HCDR3 having an amino acid sequence of SEQ ID NO: 283, the histidine on a second heavy chain comprising HCDR1 having an amino acid sequence of SEQ ID NO: 281, HCDR2 having an amino acid sequence selected from SEQ ID NOs: 282, 284 or 285, and HCDR3 having an amino acid sequence of SEQ ID NO: 283, and the cysteine residues on a light chain comprising LCDR1 having an amino acid sequence selected from SEQ ID NOs: 286 or 291, LCDR2 having an amino acid sequence selected from SEQ ID NOs: 287, 289, or 292, and LCDR3 having an amino acid sequence selected from SEQ ID NOs: 288 or 290.

In some aspects, Zn is coordinated with the cysteine residue and histidine residue on a first heavy chain comprising VH having at least 85%, 90%, 95%, 96%, 97%, 98%, 99%, or 100% sequence identity to an amino acid sequence selected from SEQ ID NOs: 293 to 297, the histidine on a second heavy chain comprising VH having at least 85%, 90%, 95%, 96%, 97%, 98%, 99%, or 100% sequence identity to an amino acid sequence selected from SEQ ID NOs: 293 to 297, and the cysteine residues on a light chain comprising VL having at least 85%, 90%, 95%, 96%, 97%, 98%, 99%, or 100% sequence identity to an amino acid sequence selected from SEQ ID NOs: 298 to 302.

In some aspects, Zn is coordinated with the cysteine residue and histidine residue on a first heavy chain comprising VH having an amino acid sequence selected from SEQ ID NOs: 293 to 297, the histidine on a second heavy chain comprising VH having an amino acid sequence selected from SEQ ID NOs: 293 to 297, and the cysteine residues on a light chain comprising VL having an amino acid sequence selected from SEQ ID NOs: 298 to 302.

In some aspects, Zn is coordinated with the cysteine residue and histidine residue on a first heavy chain of the amino acid sequence having at least 85%, 90%, 95%, 96%, 97%, 98%, 99%, or 100% sequence identity to SEQ ID NOs: 303-326, the histidine residue on a second heavy chain of the amino sequence having at least 85%, 90%, 95%, 96%, 97%, 98%, 99%, or 100% sequence identity to SEQ ID NOs: 303-326, and the cysteine residue on a light chain of the amino acid sequence having at least 85%, 90%, 95%, 96%, 97%, 98%, 99%, or 100% sequence identity to SEQ ID NOs: 327-330.

In some aspects, Zn is coordinated with the cysteine and histidine residue on a first heavy chain of the amino acid sequence of SEQ ID NOs: 303-326, the histidine residue on a second heavy chain of the amino sequence of SEQ ID NOs: 303-326, and the cysteine residue on a light chain of the amino acid sequence of SEQ ID NOs: 327-330.

### Oligonucleotide Conjugates

### Polynucleotide Acid Molecules

In some aspects, the polynucleic acid molecule described herein comprises RNA or DNA. In some cases, the polynucleic acid molecule comprises RNA. In some instances, RNA comprises short interfering RNA (siRNA), short hairpin RNA (shRNA), microRNA (miRNA), single-stranded RNA (sRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the oligonucleotide is a phosphorodiamidate morpholino oligomers (PMO), which are short single-stranded oligonucleotide analogs that are built upon a backbone of morpholine rings connected by phosphorodiamidate linkages. In some instances, the oligonucleotide comprises siRNA. In some instances, the polynucleic acid molecule comprises siRNA. In some instances, the polynucleic acid molecule comprises antisense oligonucleotides (ASO). In some instances, the polynucleic acid molecule comprises phosphorodiamidate morpholino modified antisense oligonucleotides (PMO).

In some aspects, suitable siRNAs, ASOs, and PMOs as described herein are disclosed in, e.g., U.S. Pat. Nos. 10,881,743, 11,446,387, 11,555,190, 11,912,779, 10,994,020, 11,142,767, 12,071,621, U.S. patent application Ser. No. 18/755,579, U.S. patent application Ser. No. 18/759,724, U.S. patent application Ser. No. 18/903,935, US Patent Publication 2020/0282074, U.S. patent application No. 63/574,108, U.S. patent application No. 63/574,132, U.S. patent application No. 63/712,997, U.S. patent application No. 63/718,513, U.S. Pat. No. 12,018,087, US Patent Publication No. US2021/031722, US Patent Publication No. 2024/0318177, US Patent Publication No. 2024/0209119, US Patent Publication No. 2024/0318176, US Patent Publication No. 2024/0382609, US Patent Publication No. 2024/0117356, US Patent Publication No. 2024/0325558, international patent publication No. WO2023/201324, US Patent Publication No. 2024/0110184, international patent publication No. WO2023/086864, international patent publication No. WO2024/01113, US Patent Publication No. 2024/0294921, US Patent Publication No. 2023/0346966, US Patent Publication No. 2023/0111147, US Patent Publication No. 2023/0346966, US Patent Publication No. 2023/0203180, U.S. Pat. No. 11,203,611, US Patent Publication No. 2024/0382610, international patent publication No. WO2024/238558, US Patent Publication No. 2024/374746, U.S. Pat. No. 11,920,136, US Patent Publication No. 2022/096649, international patent publication No. WO2023/225577, US Patent Publication No. 2023/364255, the content of which are hereby incorporated herein by reference in their entireties.

In some aspects, the polynucleic acid molecule is from about 8 to about 50 nucleotides in length. In some aspects, the polynucleic acid molecule is from about 10 to about 50 nucleotides in length. In some instances, the polynucleic acid molecule is from about 10 to about 30, from about 15 to about 30, from about 18 to about 25, form about 18 to about 24, from about 19 to about 23, or from about 20 to about 22 nucleotides in length.

In some aspects, the polynucleic acid molecule is about 50, 45, 40, 35, 25, 20, 19, 18, 17, 16, 15, 14, 13, 12, 11, 10 nucleotides in length. In some instances, the polynucleic acid molecule is between about 8 and about 50, between about 10 and about 50, between about 10 and about 45, between about 10 and about 40, between about 10 and about 35, between about 10 and about 30, between about 10 and about 25, between about 10 and about 20, between about 15 and about 25, between about 15 and about 30, or between about 12 and about 30 nucleotides in length.

In some aspects, the polynucleic acid molecule comprises a first polynucleotide. In some instances, the polynucleic acid molecule comprises a second polynucleotide. In some instances, the polynucleic acid molecule comprises a first polynucleotide and a second polynucleotide. In some instances, the first polynucleotide is a sense strand or passenger strand. In some instances, the second polynucleotide is an antisense strand or guide strand.

In some aspects, the polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides. In some aspects, the polynucleic acid molecule comprises a sense strand and an antisense strand, and the antisense strand includes two non-base pairing nucleotides as an overhang at the 3′-end while the sense strand has no overhang. Optionally, in such aspects, the non-base pairing nucleotides have a sequence of TT, dTdT, or UU. In some aspects, the polynucleic acid molecule comprises a sense strand and an antisense strand, and the sense strand has one or more nucleotides at the 5′-end that are complementary to the antisense sequence.

In some aspects, the polynucleic acid molecule comprises a lipophilic moiety. In some aspects, the lipophilic moiety is conjugated to the polynucleic acid. In some aspects, the lipophilic moiety is a linear or branched alkyl group. In some embodiments, the linear alkyl group is selected from the group consisting of C6, C8, C10, C12, C14, C16, C18, C20, C22, C24 and C26 hydrocarbon chain. In some aspects, the linear alkyl group is a C16 or C22 hydrocarbon chain. In some aspects, the lipophilic moiety is conjugated to a nucleotide of the polynucleotide molecule. In some aspects, the lipophilic moiety is conjugated to a sugar moiety of a nucleotide of the polynucleotide molecule at the 2′-carbon or a modification made thereof. In some aspects, the polynucleotide molecule is a single-stranded or double-stranded polynucleotide. In some aspects, the double-stranded polynucleotide is an siRNA comprising a guide strand and a passenger strand. In some aspects, the lipophilic moiety is conjugated to the passenger strand.

In some aspects, the sequence of the polynucleic acid molecule is at least 50% complementary to a target sequence described herein. In some aspects, the sequence of the polynucleic acid molecule is at least 60% complementary to a target sequence described herein. In some aspects, the sequence of the polynucleic acid molecule is at least 70% complementary to a target sequence described herein. In some aspects, the sequence of the polynucleic acid molecule is at least 80% complementary to a target sequence described herein. In some aspects, the sequence of the polynucleic acid molecule is at least 90% complementary to a target sequence described herein. In some aspects, the sequence of the polynucleic acid molecule is at least 95% complementary to a target sequence described herein. In some aspects, the sequence of the polynucleic acid molecule is at least 99% complementary to a target sequence described herein. In some instances, the sequence of the polynucleic acid molecule is 100% complementary to a target sequence described herein.

In some aspects, the sequence of the polynucleic acid molecule has five or fewer mismatches to a target sequence described herein. In some aspects, the sequence of the polynucleic acid molecule has four or fewer mismatches to a target sequence described herein. In some instances, the sequence of the polynucleic acid molecule has three or fewer mismatches to a target sequence described herein. In some cases, the sequence of the polynucleic acid molecule has two or fewer mismatches to a target sequence described herein. In some cases, the sequence of the polynucleic acid molecule has one mismatch to a target sequence described herein.

In some aspects, the specificity of the polynucleic acid molecule that hybridizes to a target sequence described herein is a 95%, 98%, 99%, 99.5%, or 100% sequence complementarity of the polynucleic acid molecule to a target sequence. In some instances, the hybridization is a high stringent hybridization condition.

In some aspects, the polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

In some aspects, the polynucleic acid molecule comprises natural or synthetic or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some aspects, nucleotide analogues or artificial nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, and disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′-O-methoxyethyl modification adds a methoxyethyl group to the 2′ hydroxyl group of the ribose moiety. Exemplary chemical structures of a 2′-O-methyl modification of an adenosine molecule and 2′-O-methoxyethyl modification of a uridine are illustrated below.

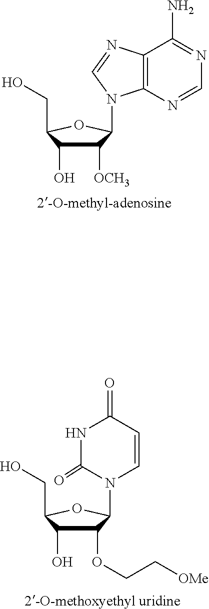

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

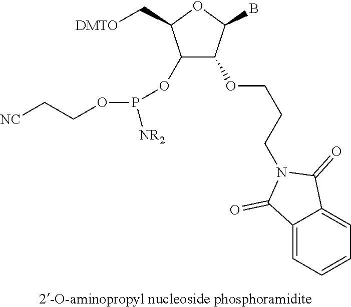

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

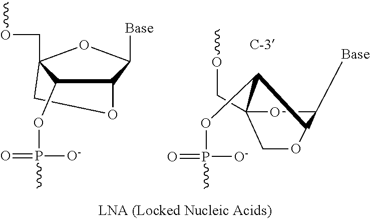

In some instances, the modification at the 2′ hydroxyl group comprises ethylene nucleic acids (ENA) such as for example 2′-4′-ethylene-bridged nucleic acid, which locks the sugar conformation into a C3′-endo sugar puckering conformation. ENA are part of the bridged nucleic acids class of modified nucleic acids that also comprises LNA. Exemplary chemical structures of the ENA and bridged nucleic acids are illustrated below.

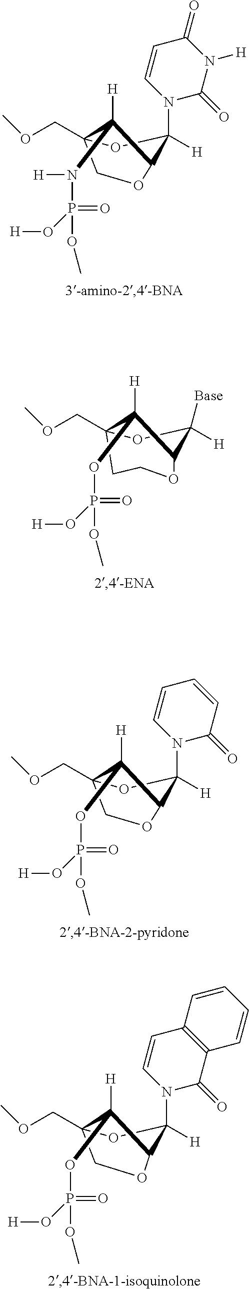

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, nucleotide analogues comprise modified bases such as, but not limited to, 5-propynyluridine, 5-propynylcytidine, 6-methyladenine, 6-methylguanine, N, N, -dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2, 4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviating from the normal sugar and phosphate structures. In some instances, the five-member ribose ring is substituted with a six-member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

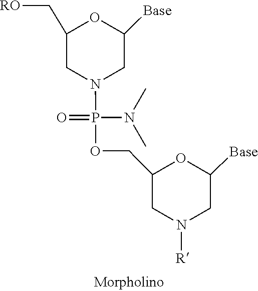

In some aspects, peptide nucleic acid (PNA) does not contain sugar ring or phosphate linkage and the bases are attached and appropriately spaced by oligoglycine-like molecules, therefore, eliminating a backbone charge.

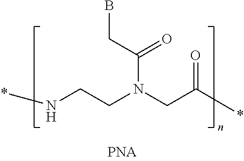

In some aspects, one or more modifications optionally occur at the internucleotide linkage. In some instances, modified internucleotide linkage include, but is not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. An exemplary PS ASO is illustrated below.

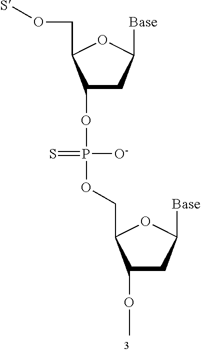

In some instances, the modification is a methyl or thiol modification such as methylphosphonate or thiolphosphonate modification. Exemplary thiolphosphonate nucleotide (left) and methylphosphonate nucleotide (right) are illustrated below.

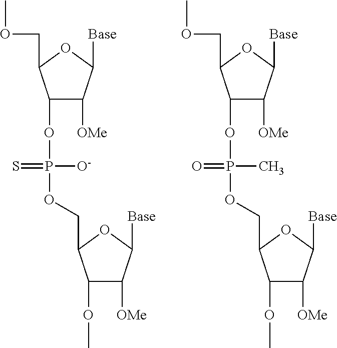

In some instances, a modified nucleotide includes, but is not limited to, 2′-fluoro N3-P5′-phosphoramidites illustrated as:

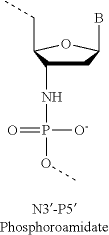

In some instances, a modified nucleotide includes, but is not limited to a 5′-vinylphosphonate modified non-natural nucleotide selected from:

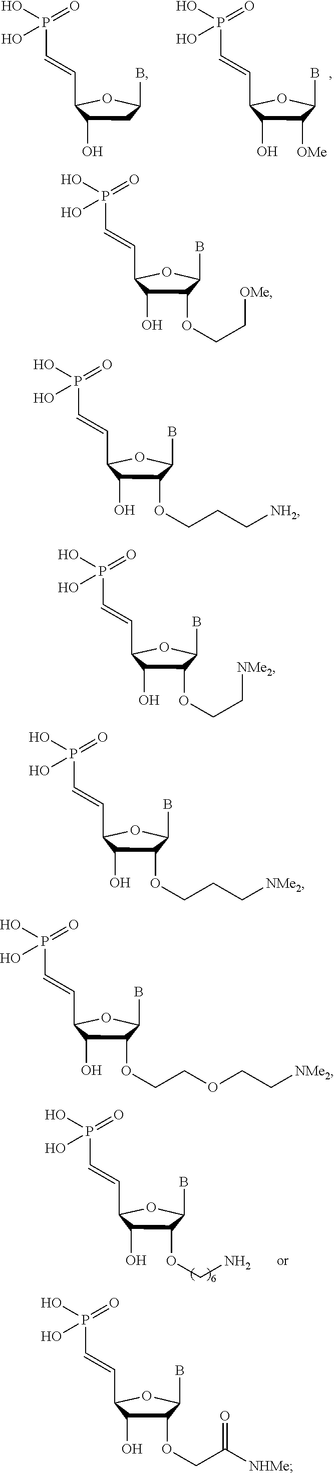

where B is a heterocyclic base moiety.

In some instances, a modified nucleotide includes, but is not limited to one 5′-vinylphosphonate modified non-natural nucleotide selected from:

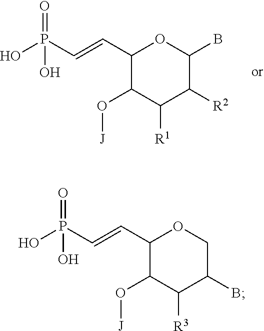

where B is a heterocyclic base moiety; R1, R2, and R3 are independently selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from:

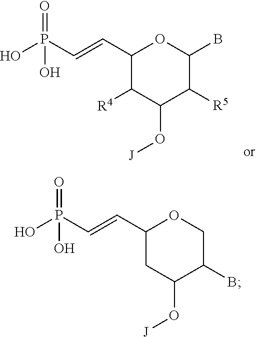

where B is a heterocyclic base moiety R4, and R5 are independently selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from:

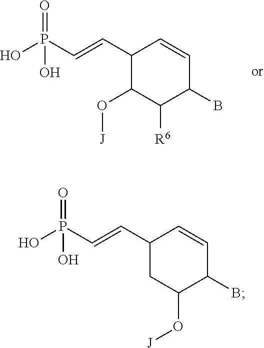

where B is a heterocyclic base moiety; R6 is selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from locked nucleic acid (LNA) or ethylene nucleic acid (ENA).

In some instances, a modified nucleotide includes, but is not limited to one 5′-vinylphosphonate modified non-natural nucleotide selected from:

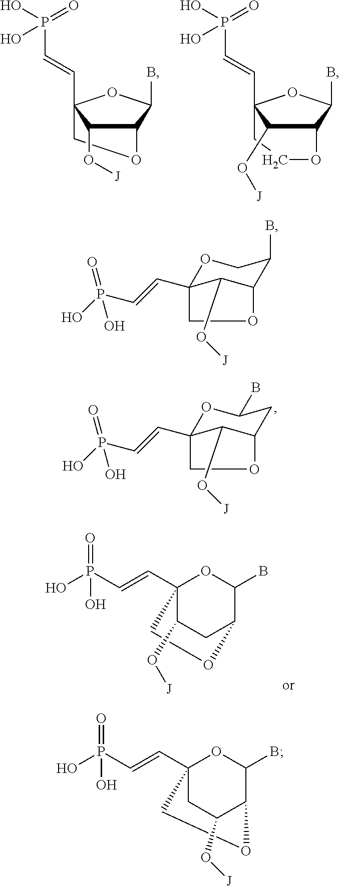

where B is a heterocyclic base moiety; and J is an internucleotide linking group linking to the adjacent nucleotide of the polynucleotide.

In some instances, a modified nucleotide includes, but is not limited to one 5′-vinylphosphonate modified non-natural nucleotide selected from:

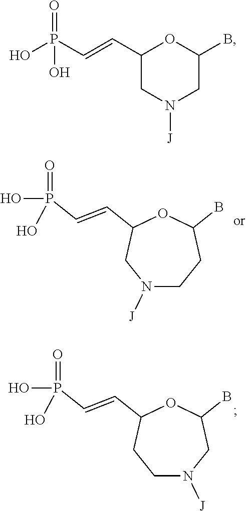

where B is a heterocyclic base moiety; and J is an internucleotide linking group linking to the adjacent nucleotide of the polynucleotide.

In some instances, a modified nucleotide includes, but is not limited to one 5′-vinylphosphonate modified non-natural nucleotide selected from:

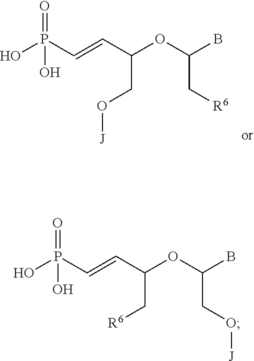

where B is a heterocyclic base moiety; R6 is selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide is:

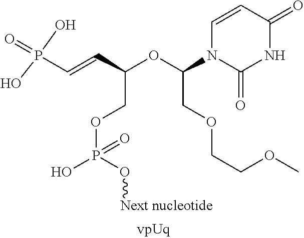

In some instances, a modified nucleotide includes, but is not limited to, hexitol nucleic acid (or 1,5′-anhydrohexitol nucleic acids (HNA)) illustrated as:

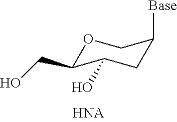

In some aspects, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some aspects, the polynucleic acid molecule comprises one or more of the artificial nucleotide analogues described herein. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification, and from about 90% to about 100% modification.

In some aspects, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modifications.

In some instances, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides.

In some instances, from about 5 to about 100% of the polynucleic acid molecule comprise the artificial nucleotide analogues described herein. In some instances, about 5%, 10%, 15%, 20%, 25%, 30%, 35%, 40%, 45%, 50%, 55%, 60%, 6%, 70%, 75%, 80%, 85%, 90%, 95% or 100% of the polynucleic acid molecule comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, 98%, 99%, or 100% of the polynucleic acid molecule comprises the artificial nucleotide analogues described herein. In additional aspects, a polynucleic acid molecule described herein is modified to increase its stability. In some embodiment, the polynucleic acid molecule is oligonucleotide (e.g., siRNA). In some instances, the polynucleic acid molecule is modified by one or more of the modifications described above to increase its stability. In some aspects, the modified nucleotide includes 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some aspects, a polynucleic acid molecule is assembled from two separate polynucleotides wherein one polynucleotide comprises the sense strand and the second polynucleotide comprises the antisense strand of the polynucleic acid molecule. In other aspects, the sense strand is connected to the antisense strand via a linker molecule, which in some instances is a polynucleotide linker or a non-nucleotide linker.

In some aspects, a polynucleic acid molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate internucleotide linkages in each strand of the polynucleic acid molecule. In some aspects, a polynucleic acid molecule comprises a sense strand and an antisense strand, and the antisense strand comprises a phosphate backbone modification at the 3′ end of the antisense strand. Alternatively and/or additionally, a polynucleic acid molecule comprises a sense strand and an antisense strand, and the sense strand comprises a phosphate backbone modification at the 5′ end of the antisense strand. In some instances, the phosphate backbone modification is a phosphorothioate. In some aspects, the sense or antisense strand has three consecutive nucleosides that are coupled via two phosphorothioate backbone.

In some instances, an asymmetric hairpin is a linear polynucleic acid molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin polynucleic acid molecule comprises an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. In some cases, the asymmetric hairpin polynucleic acid molecule also comprises a 5′-terminal phosphate group that is chemically modified. In additional cases, the loop portion of the asymmetric hairpin polynucleic acid molecule comprises nucleotides, non-nucleotides, linker molecules, or conjugate molecules.

In some aspects, an asymmetric duplex is a polynucleic acid molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex polynucleic acid molecule comprises an antisense region having length sufficient to mediate RNAi in a cell or in vitro system (e.g., about 19 to about 22 nucleotides) and a sense region having about 3 to about 18 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art.

### Lipid-Oligonucleotide (e.g., Lipid-siRNA)

In some embodiments, the lipophilic moiety is covalently conjugated to an oligonucleotide. In some embodiments, the lipophilic moiety is conjugated to a single-stranded polynucleotide molecule. In some embodiments, the lipophilic moiety is conjugated to a double-stranded polynucleotide molecule. In some embodiments, the lipophilic moiety is conjugated to an siRNA, ASO, PMO, or the like. In some embodiments, the lipophilic moiety is conjugated to an siRNA comprising a guide strand and a passenger strand. In some embodiments, the lipophilic moiety is conjugated to a guide strand of the siRNA. In some embodiments, the lipophilic moiety is conjugated to the 5′ end of the guide strand. In some embodiments, the lipophilic moiety is conjugated to the 3′ end of the guide strand. In some embodiments, the lipophilic moiety is conjugated to a passenger strand of the siRNA. In some embodiments, the lipophilic moiety is conjugated to the 5′ end of the passenger strand. In some embodiments, the lipophilic moiety is conjugated to the 3′ end of the passenger strand.

In some embodiments, the one or more lipophilic moieties are conjugated to one or more nucleotides at terminal and/or internal positions in the guide strand of siRNA. In some embodiments, the one or more lipophilic moieties are conjugated to one or more nucleotides at terminal and/or internal positions in the passenger strand of siRNA. In some embodiments, the one or more lipophilic moieties are conjugated to one or more nucleotides of the guide strand at positions selected from 1, 2, 3, 4, 5, 6, 7, 8, 9, 10, 11, 12, 13, 14, 15, 16, or 17 from the 5′ end. In some embodiments, the one or more lipophilic moieties are conjugated to one or more nucleotides of the passenger strand at positions selected from 1, 2, 3, 4, 5, 6, 7, 8, 9, 10, 11, 12, 13, 14, 15, 16, 17, 18 or 19 from the 5′ end. In some embodiments, the one or more lipophilic moieties are conjugated to one or more nucleotides of the guide strand at positions selected from 2, 3, 6, 9, 12, 17, or 19 from the 5′ end. In some embodiments, the one or more lipophilic moieties are conjugated to one or more nucleotides of the passenger strand at positions selected from 2, 3, 6, 9, 12, 17, or 19 from the 5′ end. In some embodiments, the one or more lipophilic moieties are conjugated to one or more nucleotides of the guide strand at positions selected from 3 or 6 from the 5′ end. In some embodiments, the one or more lipophilic moieties are conjugated to one or more nucleotides of the passenger strand at positions selected from 2, 3 or 6 from the 5′ end. In some embodiments, the one or more lipophilic moieties are conjugated to one or more nucleotides at terminal and/or internal positions in a single-stranded polynucleotide molecule, such as ASO or PMO.

In some embodiments, the lipophilic moiety is conjugated to a sugar ring of the nucleotide. In some embodiments, the lipophilic moiety is conjugated to a sugar moiety of a nucleotide of the polynucleotide molecule at the 2′-carbon or a modification made thereof. In some embodiments, the lipophilic moiety is conjugated to a sugar moiety of a nucleotide of the polynucleotide molecule at the 3′-carbon or a modification made thereof.

### Target Genes

In some instances, the polynucleic acid molecule targets an incorrectly processed pre-mRNA transcript which results in a disease or disorder not limited to a neuromuscular disease, a genetic disease, cancer, a hereditary disease, or a cardiovascular disease.

In some instances, a polynucleic acid molecule targets an exon that is mutated in a gene that causes a disease or disorder. Exemplary diseases or disorders include, but are not limited to, familial dysautonomia (FD), spinal muscular atrophy (SMA), medium-chain acyl-CoA dehydrogenase (MCAD) deficiency, Hutchinson-Gilford progeria syndrome (HGPS), myotonic dystrophy type I (DM1), myotonic dystrophy type II (DM2), autosomal dominant retinitis pigmentosa (RP), Duchenne muscular dystrophy (DMD), microcephalic steodysplastic primordial dwarfism type 1 (MOPD1) (Taybi-Linder syndrome (TALS), frontotemporal dementia with parkinsonism-17 (FTDP-17), Facioscapulohumeral muscular dystrophy (FSHD) Fukuyama congenital muscular dystrophy (FCMD), amyotrophic lateral sclerosis (ALS), hypercholesterolemia, and cystic fibrosis (CF). Exemplary genes that are involved in the disease or disorder include, but are not limited to, DUX4, IKBKAP, SMN2, MCAD, LMNA, DMPK, ZNF9, MAPT, FKTN, TDP-43, LDLR, CFTR, DMD, PAH, MSTN, K-Ras, GYS1, PLN, PRKAG2, and/or CNBP. In some embodiments, the gene is DMD, PAH, MSTN, HPRT1, SSB, or K-Ras.

In some embodiments, a polynucleic acid molecule hybridizes to a target sequence of an atrophy-related gene (also referred to as an atrogene). In some instances, a polynucleic acid molecule described herein hybridizes to a target sequence of an ubiquitin ligase (e.g., an E3 ubiquitin ligase or a mitochondrial ubiquitin ligase). In some instances, a polynucleic acid molecule described herein hybridizes to a target sequence of a Forkhead box transcription factor. In some instances, a polynucleic acid molecule described herein hybridizes to a target sequence of a growth factor. In some instances, a polynucleic acid molecule described herein hybridizes to a target sequence of a deubiquitinating enzyme.

In some embodiments, a polynucleic acid molecule described herein hybridizes to a target sequence of HPRT, FBXO32, TRIM63, TRAF6, FBXO30, FBXO40, NEDD4, TRIM32, MUL1, STUB1, FOXO1, FOXO3, MSTN, USP14, USP19, DDIT4, CTSL2, TGIF, MYOG, HDAC2, HDAC3, MT1L, MT1B, SSB, DMPK, DMD, DUX4, GYS1, PLN, PRKAG2, and/or CNBP.

In some embodiments, a polynucleic acid molecule conjugate or a lipid modified polynucleic acid molecule conjugate described herein hybridizes different target sequences. In some embodiments, a polynucleic acid molecule conjugate or a lipid modified polynucleic acid molecule conjugate described herein target mRNAs of different genes.

In some embodiments, the polynucleic acid molecule is a polynucleic acid disclosed in PCT/US2017/025608, PCT/US2018/012672, PCT/US2018/052289, PCT/US2018/064359, PCT/US2020/029731, PCT/US2021/022214, PCT/US2021/024303, PCT/US2022/043705, PCT/US2023/017574, PCT/US2024/035667, PCT/US2024/036259, PCT/US2024/049484, PCT/US2025/022623, and PCT/US2025/022622.

### Linkers

The linker provides a covalent bond between a binding moiety and a drug moiety. The linker provides a covalent bond between the antibody and the polynucleotide. The linker can be produced by a known method in art. For instance, it can be produced by linking an oligonucleotide with a carbon chain containing an amino group at the end, then linking the amino group to a group for introducing a thiol-reactive group, and further reacting the thiol-reactive group with Fab's of an antibody. In some aspects, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety C. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some aspects, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)_{1,3}′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide. In some embodiments, the linker comprises a bismaleimide (Bismal).

In some instances, the linker comprises a reactive functional group. In some cases, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some aspects, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some aspects, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 223), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 224), or Gly-Phe-Leu-Gly (SEQ ID NO: 225). In some instances, the linker comprises a peptide moiety such as: Val-Cit (valine-citrulline), Gly-Gly-Phe-Gly (SEQ ID NO: 223), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 224), or Gly-Phe-Leu-Gly (SEQ ID NO: 225). In some cases, the linker comprises Val-Cit. In some cases, the linker is Val-Cit.

In some aspects, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication NO. WO2015038426.

In some aspects, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some aspects, the linker is a traceless linker or a linker in which after cleavage does not leave behind a linker moiety (e.g., an atom or a linker group) to a binding moiety, a polynucleotide, a polymer, or an endosomolytic moiety. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication NOs. 2014/0127239; 2013/028919; 2014/286970; 2013/0309256; 2015/037360; or 2014/0294851; or PCT Publication NOs. WO2015057699; WO2014080251; WO2014197854; WO2014145090; or WO2014177042.

### Binding Moieties

The choice of a binding moiety for conjugation is not restricted to any specific type. In some aspects, the binding moiety is an antibody or antigen binding fragment thereof. In some aspects, the antibody or antigen binding fragment thereof may be human, murine antibody, humanized, or chimeric. In some aspects, the antibody or antigen binding fragment thereof comprises a monoclonal, or a polyclonal antibody. In some aspects, the antibody or antigen binding fragment comprises monovalent Fab, Fab′, divalent Fab2, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or antigen binding fragment thereof, or a chemically modified derivative thereof. In some aspects, the antibody or antigen binding fragment thereof is a single-arm antibody, such as Fab-Fc fusion antibody or a VHH-Fc fusion antibody. In some aspects, the antibody or antigen binding fragment thereof is a humanized antibody or antigen binding fragment thereof. In some aspects, the antibody or antigen binding fragment thereof is a murine antibody or antigen binding fragment thereof. In some aspects, the antibody or antigen binding fragment thereof is a chimeric antibody or antigen binding fragment thereof. In some aspects, the antibody or antigen binding fragment thereof is a monovalent Fab. In some aspects, the antibody or antigen binding fragment thereof is a monovalent Fab′. In some aspects, the antibody or antigen binding fragment thereof is a divalent Fab₂. In some aspects, the antibody or antigen binding fragment thereof is a single-chain variable fragment (scFv). In some aspects, the antibody or antigen binding fragment thereof is a monoclonal antibody or antigen binding fragment thereof. In some aspects, the antibody or antigen binding fragment thereof is a monoclonal antibody comprising two heavy chains and two light chains. Suitable antibody isotypes include, for example, IgG1, IgG2, IgG3, and IgG4. In some embodiments, the antibody comprises an IgG1 framework. In some embodiments, the antibody comprises an IgG2 framework. In some embodiments, the antibody comprises an IgG3 framework. In some embodiments, the antibody comprises an IgG4 framework.

In some instances, the antibody is a humanized antibody or antigen binding fragment thereof, murine antibody or antigen binding fragment thereof, chimeric antibody or antigen binding fragment thereof, monoclonal antibody or antigen binding fragment thereof, monovalent Fab, Fab′, divalent Fab2, F(ab)′₃ fragments, single-chain variable fragment (scFv), bis-scFv, (scFv)₂, diabody, minibody, nanobody, triabody, tetrabody, disulfide stabilized Fv protein (“dsFv”), single-domain antibody (sdAb), Ig NAR, camelid antibody or antigen binding fragment thereof, bispecific antibody or antigen biding fragment thereof, or a chemically modified derivative thereof. In some instances, the antibody is a humanized antibody or antigen binding fragment thereof. In some instances, the antibody is a murine antibody or antigen binding fragment thereof. In some instances, the antibody is a chimeric antibody or antigen binding fragment thereof. In some instances, the antibody is a monoclonal antibody or antigen binding fragment thereof. In some instances, the antibody is a monovalent Fab. In some instances, the antibody is a monovalent Fab′. In some instances, the antibody is a divalent Fab₂. In some instances, the antibody is a single-chain variable fragment (scFv). In some instances, the antibody is a human antibody or antigen binding fragment thereof.

In some embodiments, the antibody is a bispecific antibody or antigen binding fragment thereof. In some instances, the bispecific antibody is a trifunctional antibody or a bispecific mini-antibody. In some cases, the bispecific antibody is a trifunctional antibody. In some instances, the trifunctional antibody is a full-length monoclonal antibody comprising binding sites for two different antigens. Exemplary trifunctional antibodies include catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (R05520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), and TRBS07 (Ektomab; targets GD2/CD3; Trion Research Gmbh). Additional exemplary trifunctional antibodies include mAb² from F-star Biotechnology Ltd. In some instances, the antibody is a bispecific trifunctional antibody. In some embodiments, the antibody is a bispecific trifunctional antibody selected from: catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (R05520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), TRBS07 (Ektomab; targets GD2/CD3; Trion Research Gmbh), and a mAb² from F-star Biotechnology Ltd.

In some cases, the bispecific antibody is a bispecific mini-antibody. In some instances, the bispecific mini-antibody comprises divalent Fab2, F(ab)′₃ fragments, bis-scFv, (scFv)₂, diabody, minibody, triabody, tetrabody or a Bi-specific T-cell Engager (BiTE). In some embodiments, the Bi-specific T-cell Engager is a fusion protein that contains two single-chain variable fragments (scFvs) in which the two scFvs target epitopes of two different antigens. Exemplary bispecific mini-antibodies include, but are not limited to, DART (dual-affinity re-targeting platform; MacroGenics), blinatumomab (MT103 or AMG103; which targets CD19/CD3; Micromet), MT111 (targets CEA/CD3; Micromet/Amgen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the antibody is a bispecific mini-antibody. In some instances, the antibody is a bispecific Fab₂. In some instances, the antibody is a bispecific F(ab)′₃ fragment. In some cases, the antibody is a bispecific bis-scFv. In some cases, the antibody is a bispecific (scFv)₂. In some embodiments, the antibody is a bispecific diabody. In some embodiments, the antibody is a bispecific minibody. In some embodiments, the antibody is a bispecific triabody. In other embodiments, the antibody is a bispecific tetrabody. In other embodiments, the antibody is a Bi-specific T-cell Engager (BiTE). In additional embodiments, the antibody is a bispecific mini-antibody selected from: DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the antibody is a trispecific antibody. In some instances, the trispecific antibody comprises F(ab)′₃ fragments or a triabody. In some instances, the antibody is a trispecific F(ab)′₃ fragment. In some cases, the antibody is a triabody. In some embodiments, the antibody is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some embodiments, the antibody is an antibody or antigen binding fragment thereof that recognizes a cell surface protein. In some instances, the cell surface protein is an antigen expressed by a cancerous cell. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAPIB, SPAPIC), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) and the like. In some embodiments, the antibody is an antibody or antigen binding fragment thereof that recognizes a glycoprotein on the surface of RSV (Respiratory Syncytial Virus). In some embodiments, the antibody is an antibody or antigen binding fragment thereof that recognizes a fusion (F) glycoprotein on the surface of RSV. In some embodiments, the antibody is an antibody or antigen binding fragment thereof is Palivizumab.

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71 (transferrin receptor), CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some instances, the antibody is an antibody or antigen binding fragment thereof that recognizes a cancer antigen. In some instances, the antibody is an antibody or antigen binding fragment thereof that recognizes alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAPIB, SPAPIC), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MCP1 (CCL2), MDP, macrophage inhibitory factor (MIF), MPF (SMR, MSLN), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) or a combination thereof.

In some instances, the antibody is an antibody or antigen binding fragment thereof that recognizes a CD cell surface marker. In some instances, the antibody is an antibody or antigen binding fragment thereof that recognizes CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71 (transferrin receptor), CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), or a combination thereof.

In some embodiments, the antibody or antigen binding fragment thereof comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hulD10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAbl7-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), namatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), zatuximab, and the like. In some embodiments, the antibody or antigen binding fragment thereof is trastuzumab. In some embodiments, the antibody or antigen binding fragment thereof is palivizumab.

In some embodiments, the antibody comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hulD10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAbl7-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-STO1, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), or zatuximab. In some embodiments, the binding moiety A is zalutumumab (HuMax-EFGr, by Genmab). In some embodiments, the binding moiety is trastuzumab. In some embodiments, the binding moiety is palivizumab.

In some aspects, exemplary antibodies include, but are not limited to, anti-transferrin receptor antibodies. In some instances, the antibody is an anti-transferrin receptor (anti-CD71) antibody.

In some aspects, where the antibody is an anti-transferrin receptor (anti-CD71) antibody, the anti-transferrin antibody specifically binds to a transferrin receptor (TfR), preferably, specifically binds to transferrin receptor 1 (TfR1), or more preferably, specifically binds to human transferrin receptor 1 (TfR1, or human CD71).

In some instances, the anti-transferrin receptor antibody comprises a variable heavy chain (VH) region and a variable light chain (VL) region, wherein the VH region comprises HCDR1 sequence comprising SEQ ID NO: 281; HCDR2 sequence EINPIX₁GRSNYAX₂KFQG (SEQ ID NO: 226), wherein X₁ is selected from N or Q and X₂ is selected from Q or E; and HCDR3 sequence comprising SEQ ID NO: 283.

In some aspects, the VH region of the anti-transferrin receptor antibody comprises HCDR1, HCDR2, and HCDR3 sequences selected from Table 1.

TABLE 1

| | | SEQ | | SEQ | | SEQ |
|---|---|---|---|---|---|---|
| | | ID | | ID | | ID |
| Name | HCDR1 | NO: | HCDR2 | NO: | HCDR3 | NO: |
| 13E4_VH1 | YTFTN | 281 | EINPINGRS | 282 | GTRAMHY | 283 |
| | YWMH | | NYAQKFQG | | | |
| 13E4_VH2* | YTFTN | 281 | EINPINGRS | 284 | GTRAMHY | 283 |
| | YWMH | | NYAEKFQG | | | |
| 13E4_VH3 | YTFTN | 281 | EINPIQGRS | 285 | GTRAMHY | 283 |
| | YWMH | | NYAEKFQG | | | |

*13E4_VH2 shares the same HCDR1, HCDR2, and HCDR3 sequences with anti-transferrin receptor antibody 13E4_VH4

In some aspects, the VH region comprises HCDR1 sequence comprising SEQ ID NO: 281; HCDR2 sequence comprising SEQ ID NO: 282, 284, or 285; and HCDR3 sequence comprising SEQ ID NO: 283. In some instances, the VH region comprises HCDR1 sequence comprising SEQ ID NO: 281, HCDR2 sequence comprising SEQ ID NO: 282, and HCDR3 sequence comprising SEQ ID NO: 283. In some instances, the VH region comprises HCDR1 sequence comprising SEQ ID NO: 281, HCDR2 sequence comprising SEQ ID NO: 284, and HCDR3 sequence comprising SEQ ID NO: 283. In some instances, the VH region comprises HCDR1 sequence comprising SEQ ID NO: 281, HCDR2 sequence comprising SEQ ID NO: 285, and HCDR3 sequence comprising SEQ ID NO: 283.

In some aspects, the VL region of the anti-transferrin receptor antibody comprises LCDR1 sequence RTSENIYX₃NLA (SEQ ID NO: 227), LCDR2 sequence AX₄TNLAX₅ (SEQ ID NO: 228), and LCDR3 sequence QHFWGTPLTX₆ (SEQ ID NO: 229), wherein X₃ is selected from N or S, X₄ is selected from A or G, X₅ is selected from D or E, and X₆ is present or absence, and if present, is F.

In some aspects, the VL region of the anti-transferrin receptor antibody comprises LCDR1, LCDR2, and LCDR3 sequences selected from Table 2.

TABLE 2

| | | SEQ | | SEQ | | SEQ |
|---|---|---|---|---|---|---|
| | | ID | | ID | | ID |
| Name | LCDR1 | NO: | LCDR2 | NO: | LCDR3 | NO: |
| 13E4_VL1* | RTSENI | 286 | AATN | 287 | QHFWG | 288 |
| | YNNLA | | LAD | | TPLT | |
| 13E4_VL3 | RTSENI | 286 | AATN | 289 | QHFWG | 290 |
| | YNNLA | | LAE | | TPLTF | |
| 13E4_VL4 | RTSENI | 291 | AGTN | 292 | QHFWG | 290 |
| | YSNLA | | LAD | | TPLTF | |

*13E4_VL1 shares the same LCDR1, LCDR2, and LCDR3 sequences with anti-transferrin receptor antibody 13E4_VL2

In some instances, the VL region comprises LCDR1 sequence RTSENIYX3NLA (SEQ ID NO: 227), LCDR2 sequence comprising SEQ ID NO: 287, 289, or 292, and LCDR3 sequence comprising SEQ ID NO: 288 or 290, wherein X3 is selected from N or S.

In some instances, the VL region comprises LCDR1 sequence comprising SEQ ID NO: 286 or 291, LCDR2 sequence AX4TNLAX5 (SEQ ID NO: 228), and LCDR3 sequence comprising SEQ ID NO: 288 or 290, wherein X4 is selected from A or G, and X5 is selected from D or E.

In some instances, the VL region comprises LCDR1 sequence comprising SEQ ID NO: 286 or 291, LCDR2 sequence SEQ ID NO: 287, 289, or 292, and LCDR3 sequence QHFWGTPLTX6 (SEQ ID NO: 229), wherein X₆ is present or absence, and if present, is F.

In some instances, the VL region comprises LCDR1 sequence comprising SEQ ID NO: 286, LCDR2 sequence AATNLAX5 (SEQ ID NO: 230), and LCDR3 sequence QHFWGTPLTX₆ (SEQ ID NO: 229), wherein X5 is selected from D or E and X6 is present or absence, and if present, is F.

In some instances, the VL region comprises LCDR1 sequence comprising SEQ ID NO: 286, LCDR2 sequence comprising SEQ ID NO: 287, and LCDR3 sequence comprising SEQ ID NO: 288.

In some instances, the VL region comprises LCDR1 sequence comprising SEQ ID NO: 286, LCDR2 sequence comprising SEQ ID NO: 289, and LCDR3 sequence comprising SEQ ID NO: 290.

In some instances, the VL region comprises LCDR1 sequence comprising SEQ ID NO: 291, LCDR2 sequence comprising SEQ ID NO: 292, and LCDR3 sequence comprising SEQ ID NO: 290.

In some aspects, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence RTSENIYX3NLA (SEQ ID NO: 227), LCDR2 sequence AX4TNLAX5 (SEQ ID NO: 228), and LCDR3 sequence QHFWGTPLTX6 (SEQ ID NO: 229), wherein X3 is selected from N or S, X4 is selected from A or G, X5 is selected from D or E, and X6 is present or absence, and if present, is F.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence RTSENIYX3NLA (SEQ ID NO: 227), LCDR2 sequence comprising SEQ ID NO: 287, 289, or 292, and LCDR3 sequence comprising SEQ ID NO: 288 or 290, wherein X3 is selected from N or S.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence comprising SEQ ID NO: 286 or 291, LCDR2 sequence AX4TNLAX5 (SEQ ID NO: 228), and LCDR3 sequence comprising SEQ ID NO: 288 or 290, wherein X4 is selected from A or G, and X5 is selected from D or E.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX6 (SEQ ID NO: 229), wherein X6 is present or absence, and if present, is F.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence comprising SEQ ID NO: 286, LCDR2 sequence AATNLAX5 (SEQ ID NO: 230), and LCDR3 sequence QHFWGTPLTX6 (SEQ ID NO: 229), wherein X5 is selected from D or E and X6 is present or absence, and if present, is F.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO: 290.

In some instances, the anti-transferrin receptor antibody comprises a VH region and a VL region, wherein the VH region comprises HCDR1 sequence comprising SEQ ID NO: 281; HCDR2 sequence EINPIX1GRSNYAX2KFQG (SEQ ID NO: 226), wherein X1 is selected from N or Q and X2 is selected from Q or E; and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence RTSENIYX3NLA (SEQ ID NO: 227), LCDR2 sequence comprising SEQ ID NO: 287, 289, or 292, and LCDR3 sequence comprising SEQ ID NO: 288 or 290, wherein X3 is selected from N or S.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286 or 291, LCDR2 sequence AX4TNLAX5 (SEQ ID NO: 228), and LCDR3 sequence comprising SEQ ID NO: 288 or 290, wherein X4 is selected from A or G, and X5 is selected from D or E.

In some instances, the anti-transferrin receptor antibody comprises a VH region and a VL region, in which the VH region comprises HCDR1 sequence comprising SEQ ID NO: 281, HCDR2 sequence comprising SEQ ID NO: 2, and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX6 (SEQ ID NO: 229), wherein X6 is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286, LCDR2 sequence AATNLAX5 (SEQ ID NO: 230), and LCDR3 sequence QHFWGTPLTX6 (SEQ ID NO: 229), wherein X5 is selected from D or E and X6 is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286, LCDR2 sequence comprising SEQ ID NO: 9, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence RTSENTYX3NLA (SEQ ID NO: 227), LCDR2 sequence comprising SEQ ID NO: 287, 289, or 292, and LCDR3 sequence comprising SEQ ID NO: 288 or 290, wherein X3 is selected from N or S.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286 or 291, LCDR2 sequence AX4TNLAX5 (SEQ ID NO: 228), and LCDR3 sequence comprising SEQ ID NO: 288 or 290, wherein X4 is selected from A or G, and X5 is selected from D or E.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX6 (SEQ ID NO: 229), wherein X6 is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286, LCDR2 sequence AATNLAX5 (SEQ ID NO: 230), and LCDR3 sequence QHFWGTPLTX6 (SEQ ID NO: 229), wherein X5 is selected from D or E and X6 is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO:290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence RTSENIYX3NLA (SEQ ID NO: 227), LCDR2 sequence comprising SEQ ID NO: 287, 289, or 29, and LCDR3 sequence comprising SEQ ID NO: 288 or 290, wherein X3 is selected from N or S.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286 or 291, LCDR2 sequence AX4TNLAX5 (SEQ ID NO: 228), and LCDR3 sequence comprising SEQ ID NO: 288 or 290, wherein X4 is selected from A or G, and X5 is selected from D or E.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX6 (SEQ ID NO: 229), wherein X6 is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283 and the VL region comprises LCDR1 sequence comprising SEQ ID NO: 286, LCDR2 sequence AATNLAX5 (SEQ ID NO: 230), and LCDR3 sequence QHFWGTPLTX6 (SEQ ID NO: 229), wherein X5 is selected from D or E and X6 is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some aspects, the anti-transferrin receptor antibody comprises a VH region and a VL region in which the sequence of the VH region comprises about 80%, 85%, 90%, 95%, 96% 97%, 98%, 99%, or 100% sequence identity to SEQ ID NOs: 293-296 and the sequence of the VL region comprises about 80%, 85%, 90%, 95%, 96% 97%, 98%, 99%, or 100% sequence identity to SEQ ID NOs: 298-301.

In some aspects, the VH region comprises a sequence selected from SEQ ID NOs: 293-297 (Table 3) and the VL region comprises a sequence selected from SEQ ID NOs: 298-302 (Table 4). The underlined regions in Table 3 and Table 4 denote the respective CDR1, CDR2, or CDR3 sequence.

TABLE 3

| | | SEQ ID |
|---|---|---|
| NAME | VH SEQUENCE | NO: |
| 13E4_VH1 | QVQLVQSGAEVKKPGASVKVSCKASGYTFTNYWMHWVRQAP | 293 |
| | GQGLEWMGEINPINGRSNYAQKFQGRVTLTVDTSISTAYME | |
| | LSRLRSDDTAVYYCARGTRAMHYWGQGTLVTVSS | |
| 13E4_VH2 | QVQLVQSGAEVKKPGASVKVSCKASGYTFTNYWMHWVRQAP | 294 |
| | GQGLEWIGEINPINGRSNYAEKFQGRVTLTVDTSSSTAYMELSR | |
| | LRSDDTAVYYCARGTRAMHYWGQGTLVTVSS | |
| 13E4_VH3 | QVQLVQSGAEVKKPGASVKVSCKASGYTFTNYWMHWVRQAP | 295 |
| | GQGLEWMGEINPIQGRSNYAEKFQGRVTLTVDTSSSTAYMELS | |
| | SLRSEDTATYYCARGTRAMHYWGQGTLVTVSS | |
| 13E4_VH4 | QVQLVQSGAEVKKPGASVKVSCKASGYTFTNYWMHWVRQAP | 296 |
| | GQGLEWMGEINPINGRSNYAEKFQGRVTLTVDTSSSTAYMELS | |
| | SLRSEDTATYYCARGTRAMHYWGQGTLVTVSS | |
| 13E4_VH | QVQLQQPGAELVKPGASVKLSCKASGYTFTNYWMHWVKQRP | 297 |
| | GQGLEWIGEINPINGRSNYGERFKTKATLTVDKSSSTAYMQLSS | |
| | LTSEDSAVYYCARGTRAMHYWGQGTSVTVSS | |

TABLE 4

| | | SEQ ID |
|---|---|---|
| NAME | VL SEQUENCE | NO: |
| 13E4_VL1 | DIQMTQSPSSLSASVGDRVTITCRTSENIYNNLAWYQQKPGKSP | 298 |
| | KLLIYAATNLADGVPSRFSGSGSGTDYTLTISSLQPEDFATYYCQ | |
| | HFWGTPLTFGGGTKVEIK | |
| 13E4_VL2 | DIQMTQSPSSLSASVGDRVTITCRTSENIYNNLAWYQQKPGKAP | 299 |
| | KLLIYAATNLADGVPSRFSGSGSGTDYTLTISSLQPEDFATYYCQ | |
| | HFWGTPLTFGGGTKVEIK | |
| 13E4_VL3 | DIQMTQSPSSLSASVGDRVTITCRTSENIYNNLAWYQQKPGKAP | 300 |
| | KLLIYAATNLAEGVPSRFSGSGSGTDYTLTISSLQPEDFATYYCQ | |
| | HFWGTPLTFGGGTKVEIK | |
| 13E4_VL4 | DIQMTQSPSSLSASVGDRVTITCRTSENIYSNLAWYQQKPGKAP | 301 |
| | KLLIYAGTNLADGVPSRFSGSGSGTDYTLTISSLQPEDFANYYC | |
| | QHFWGTPLTFGGGTKVEIK | |
| 13E4_VL | DIQMTQSPASLSVSVGETVTITCRTSENIYNNLAWYQQKQGKSP | 302 |
| | QLLVYAATNLADGVPSRFSGSGSGTQYSLKINSLQSEDFGNYY | |
| | CQHFWGTPLTFGAGTKLELK | |

In some aspects, the anti-transferrin receptor antibody comprises a VH region and a VL region as illustrated in Table 5.

TABLE 5

| | | 13E4_VH2 | | 13E4_VH4 |
|---|---|---|---|---|
| | 13E4_VH1 | (SEQ ID NO: | 13E4_VH3 | (SEQ ID NO: |
| | (SEQ ID NO: 293) | 294) | (SEQ ID NO: 295) | 296) |
| 13E4_VL1 | SEQ ID NO: 293 + | SEQ ID NO: | SEQ ID NO: 295 + | SEQ ID NO: |
| (SEQ ID NO: | SEQ ID NO: 298 | 294 + SEQ ID NO: | SEQ ID NO: 298 | 296 + |
| 298) | | 298 | | SEQ ID NO: |
| | | | | 298 |
| 13E4_VL2 | SEQ ID NO: 293 + | SEQ ID NO: 294 + | SEQ ID NO: 295 + | SEQ ID NO: |
| (SEQ ID NO: | SEQ ID NO: 299 | SEQ ID NO: | SEQ ID NO: 299 | 296 + |
| 299) | | 299 | | SEQ ID NO: |
| | | | | 299 |
| 13E4_VL3 | SEQ ID NO: 293 + | SEQ ID NO: 294 + | SEQ ID NO: 295 + | SEQ ID NO: |
| (SEQ ID NO: | SEQ ID NO: 300 | SEQ ID NO: | SEQ ID NO: 300 | 296 + |
| 300) | | 300 | | SEQ ID NO: |
| | | | | 300 |
| 13E4_VL4 | SEQ ID NO: 293 + | SEQ ID NO: 294 + | SEQ ID NO: 295 + | SEQ ID NO: |
| (SEQ ID NO: | SEQ ID NO: 301 | SEQ ID NO: | SEQ ID NO: 301 | 296 + |
| 301) | | 301 | | SEQ ID NO: |
| | | | | 301 |

In some aspects, an anti-transferrin receptor antibody described herein comprises an IgG framework, an IgA framework, an IgE framework, or an IgM framework. In some instances, the anti-transferrin receptor antibody comprises an IgG framework (e.g., IgG1, IgG2, IgG3, or IgG4). In some cases, the anti-transferrin receptor antibody comprises an IgG1 framework. In some cases, the anti-transferrin receptor antibody comprises an IgG2 (e.g., an IgG2a or IgG2b) framework. In some cases, the anti-transferrin receptor antibody comprises an IgG2a framework. In some cases, the anti-transferrin receptor antibody comprises an IgG2b framework. In some cases, the anti-transferrin receptor antibody comprises an IgG3 framework. In some cases, the anti-transferrin receptor antibody comprises an IgG4 framework.

In some cases, an anti-transferrin receptor antibody comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional instances, the one or more mutations are to modulate glycosylation.

In some aspects, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some instances, the Fc region comprises a mutation at residue position L234, L235, D265, N297, K322, L328, or P329, or a combination thereof. In some instances, the mutations comprise L234 and L235 in combination with a mutation at residue position K322, L328, or P329. In some cases, the Fc region comprises mutations at L234, L235, and K322. In some cases, the Fc region comprises mutations at L234, L235, and L328. In some cases, the Fc region comprises mutations at L234, L235, and P329. In some cases, the Fc region comprises mutations at D265 and N297. In some cases, the residue position is in reference to IgG1.

In some instances, the Fc region comprises L234A, L235A, D265A, N297G, K322G, L328R, or P329G, or a combination thereof. In some instances, the Fc region comprises L234A and L235A in combination with K322G, L328R, or P329G. In some cases, the Fc region comprises L234A, L235A, and K322G. In some cases, the Fc region comprises L234A, L235A, and L328R. In some cases, the Fc region comprises L234A, L235A, and P329G. In some cases, the Fc region comprises D265A and N297G. In some cases, the residue position is in reference to IgG1.

In some instances, the Fc region comprises a mutation at residue position L235, L236, D265, N297, K322, L328, or P329, or a combination of the mutations. In some instances, the Fc region comprises mutations at L235 and L236. In some instances, the Fc region comprises mutations at L235 and L236 in combination with a mutation at residue position K322, L328, or P329. In some cases, the Fc region comprises mutations at L235, L236, and K322. In some cases, the Fc region comprises mutations at L235, L236, and L328. In some cases, the Fc region comprises mutations at L235, L236, and P329. In some cases, the Fc region comprises mutations at D265 and N297. In some cases, the residue position is in reference to IgG2b.

In some aspects, the Fc region comprises L235A, L236A, D265A, N297G, K322G, L328R, or P329G, or a combination thereof. In some instances, the Fc region comprises L235A and L236A. In some instances, the Fc region comprises L235A and L236A in combination with K322G, L328R, or P329G. In some cases, the Fc region comprises L235A, L236A, and K322G. In some cases, the Fc region comprises L235A, L236A, and L328R. In some cases, the Fc region comprises L235A, L236A, and P329G. In some cases, the Fc region comprises D265A and N297G. In some cases, the residue position is in reference to IgG2b.

In some aspects, the Fc region comprises a mutation at residue position L233, L234, D264, N296, K321, L327, or P328, wherein the residues correspond to positions 233, 234, 264, 296, 321, 327, and 328 of SEQ ID NO: 303. In some instances, the Fc region comprises mutations at L233 and L234. In some instances, the Fc region comprises mutations at L233 and L234 in combination with a mutation at residue position K321, L327, or P328. In some cases, the Fc region comprises mutations at L233, L234, and K321. In some cases, the Fc region comprises mutations at L233, L234, and L327. In some cases, the Fc region comprises mutations at L233, L234, and K321. In some cases, the Fc region comprises mutations at L233, L234, and P328. In some instances, the Fc region comprises mutations at D264 and N296. In some cases, equivalent positions to residue L233, L234, D264, N296, K321, L327, or P328 in an IgG1, IgG2, IgG3, or IgG4 framework are contemplated. In some cases, mutations to a residue that corresponds to residue L233, L234, D264, N296, K321, L327, or P328 of SEQ ID NO: 23 in an IgG1, IgG2, or IgG4 framework are also contemplated.

In some aspects, the Fc region comprises L233A, L234A, D264A, N296G, K321G, L327R, or P328G, wherein the residues correspond to positions 233, 234, 264, 296, 321, 327, and 328 of SEQ ID NO: 303. In some instances, the Fc region comprises L233A and L234A. In some instances, the Fc region comprises L233A and L234A in combination with K321G, L327R, or P328G. In some cases, the Fc region comprises L233A, L234A, and K321G. In some cases, the Fc region comprises L233A, L234A, and L327R. In some cases, the Fe region comprises L233A, L234A, and K321G. In some cases, the Fc region comprises L233A, L234A, and P328G. In some instances, the Fc region comprises D264A and N296G.

In some aspects, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some aspects, an anti-transferrin receptor antibody described herein is a full-length antibody, comprising a heavy chain (HC) and a light chain (LC). In some cases, the heavy chain (HC) comprises a sequence selected from Table 6. In some cases, the light chain (LC) comprises a sequence selected from Table 7. The underlined region denotes the respective CDRs.

TABLE 6

| | | SEQ ID |
|---|---|---|
| NAME | HC SEQUENCE | NO: |
| 13E4_VH1 | QVQLVQSGAEVKKPGASVKVSCKASGYTFTNYWMHWV | 303 |
| | RQAPGQGLEWMGEINPINGRSNYAQKFQGRVTLTVDTSI | |
| | STAYMELSRLRSDDTAVYYCARGTRAMHYWGQGTLVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH1_a | QVQLVQSGAEVKKPGASVKVSCKASGYTFTNYWMHWV | 304 |
| | RQAPGOGLEWMGEINPINGRSNYAQKFQGRVTLTVDTSI | |
| | STAYMELSRLRSDDTAVYYCARGTRAMHYWGQGTLVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKRVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALPAPIEKTISKAKGQPREPQVYT | |
| | LPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 13E4_VH1_b | QVQLVQSGAEVKKPGASVKVSCKASGYTFTNYWMHWV | 305 |
| | RQAPGQGLEWMGEINPINGRSNYAQKFQGRVTLTVDTSI | |
| | STAYMELSRLRSDDTAVYYCARGTRAMHYWGQGTLVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKRVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCGVSNKALPAPIEKTISKAKGQPREPQVYT | |
| | LPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 13E4_VH1_c | QVQLVQSGAEVKKPGASVKVSCKASGYTFTNYWMHWV | 306 |
| | RQAPGQGLEWMGEINPINGRSNYAQKFQGRVTLTVDTSI | |
| | STAYMELSRLRSDDTAVYYCARGTRAMHYWGQGTLVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKRVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKARPAPIEKTISKAKGQPREPQVYT | |
| | LPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 13E4_VH1_d | QVQLVQSGAEVKKPGASVKVSCKASGYTFTNYWMHWV | 307 |
| | RQAPGQGLEWMGEINPINGRSNYAQKFQGRVTLTVDTSI | |
| | STAYMELSRLRSDDTAVYYCARGTRAMHYWGQGTLVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKRVEPKSCDKTHTCPPCPAPE | |
| | AAGGPSVFLFPPKPKDTLMISRTPEVTCVVVDVSHEDPEV | |
| | KFNWYVDGVEVHNAKTKPREEQYNSTYRVVSVLTVLHQ | |
| | DWLNGKEYKCKVSNKALGAPIEKTISKAKGQPREPQVYT | |
| | LPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 13E4_VH1_e | QVQLVQSGAEVKKPGASVKVSCKASGYTFTNYWMHWV | 308 |
| | RQAPGQGLEWMGEINPINGRSNYAQKFQGRVTLTVDTSI | |
| | STAYMELSRLRSDDTAVYYCARGTRAMHYWGQGTLVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKRVEPKSCDKTHTCPPCPAPEL | |
| | LGGPSVFLFPPKPKDTLMISRTPEVTCVVVAVSHEDPEVK | |
| | FNWYVDGVEVHNAKTKPREEQYGSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH2 | QVQLVQSGAEVKKPGASVKVSCKASGYTFTNYWMHWV | 309 |
| | RQAPGQGLEWIGEINPINGRSNYAEKFQGRVTLTVDTSSS | |
| | TAYMELSRLRSDDTAVYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH2_a | QVQLVQSGAEVKKPGASVKVSCKASGYTFTNYWMHWV | 310 |
| | RQAPGQGLEWIGEINPINGRSNYAEKFQGRVTLTVDTSSS | |
| | TAYMELSRLRSDDTAVYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH2_b | QVQLVQSGAEVKKPGASVKVSCKASGYTFTNYWMHWV | 311 |
| | RQAPGQGLEWIGEINPINGRSNYAEKFQGRVTLTVDTSSS | |
| | TAYMELSRLRSDDTAVYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCGVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH2_c | QVQLVQSGAEVKKPGASVKVSCKASGYTFTNYWMHWV | 312 |
| | RQAPGQGLEWIGEINPINGRSNYAEKFQGRVTLTVDTSSS | |
| | TAYMELSRLRSDDTAVYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKARPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH2_d | QVQLVQSGAEVKKPGASVKVSCKASGYTFTNYWMHWV | 313 |
| | RQAPGQGLEWIGEINPINGRSNYAEKFQGRVTLTVDTSSS | |
| | TAYMELSRLRSDDTAVYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALGAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH2_e | QVQLVQSGAEVKKPGASVKVSCKASGYTFTNYWMHWV | 314 |
| | RQAPGQGLEWIGEINPINGRSNYAEKFQGRVTLTVDTSSS | |
| | TAYMELSRLRSDDTAVYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVAVSHEDPEVKF | |
| | NWYVDGVEVHNAKTKPREEQYGSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH3 | QVQLVQSGAEVKKPGASVKVSCKASGYTFTNYWMHWV | 315 |
| | RQAPGQGLEWMGEINPIQGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH3_a | QVQLVQSGAEVKKPGASVKVSCKASGYTFTNYWMHWV | 316 |
| | RQAPGQGLEWMGEINPIQGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH3_b | QVQLVQSGAEVKKPGASVKVSCKASGYTFTNYWMHWV | 317 |
| | RQAPGQGLEWMGEINPIQGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCGVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH3_c | QVQLVQSGAEVKKPGASVKVSCKASGYTFTNYWMHWV | 318 |
| | RQAPGQGLEWMGEINPIQGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKARPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH3_d | QVQLVQSGAEVKKPGASVKVSCKASGYTFTNYWMHWV | 319 |
| | RQAPGQGLEWMGEINPIQGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALGAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH3_e | QVQLVQSGAEVKKPGASVKVSCKASGYTFTNYWMHWV | 320 |
| | RQAPGQGLEWMGEINPIQGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVAVSHEDPEVKF | |
| | NWYVDGVEVHNAKTKPREEQYGSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH4 | QVQLVQSGAEVKKPGASVKVSCKASGYTFTNYWMHWV | 321 |
| | RQAPGQGLEWMGEINPINGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH4_a | QVQLVQSGAEVKKPGASVKVSCKASGYTFTNYWMHWV | 322 |
| | RQAPGQGLEWMGEINPINGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH4_b | QVQLVQSGAEVKKPGASVKVSCKASGYTFTNYWMHWV | 323 |
| | RQAPGQGLEWMGEINPINGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCGVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH4_c | QVQLVQSGAEVKKPGASVKVSCKASGYTFTNYWMHWV | 324 |
| | RQAPGQGLEWMGEINPINGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKARPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH4_d | QVQLVQSGAEVKKPGASVKVSCKASGYTFTNYWMHWV | 325 |
| | RQAPGQGLEWMGEINPINGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALGAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 13E4_VH4_e | QVQLVQSGAEVKKPGASVKVSCKASGYTFTNYWMHWV | 326 |
| | RQAPGQGLEWMGEINPINGRSNYAEKFQGRVTLTVDTSS | |
| | STAYMELSSLRSEDTATYYCARGTRAMHYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPELL | |
| | GGPSVFLFPPKPKDTLMISRTPEVTCVVVAVSHEDPEVKF | |
| | NWYVDGVEVHNAKTKPREEQYGSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |

TABLE 7

| | | SEQ ID |
|---|---|---|
| NAME | LC SEQUENCE | NO: |
| 13E4_VL1 | DIQMTQSPSSLSASVGDRVTITCRTSENIYNNLAWYQQKP | 327 |
| | GKSPKLLIYAATNLADGVPSRFSGSGSGTDYTLTISSLQPE | |
| | DFATYYCQHFWGTPLTFGGGTKVEIKRTVAAPSVFIFPPSD | |
| | EQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| 13E4_VL2 | DIQMTQSPSSLSASVGDRVTITCRTSENIYNNLAWYQQKP | 328 |
| | GKAPKLLIYAATNLADGVPSRFSGSGSGTDYTLTISSLQPE | |
| | DFATYYCQHFWGTPLTFGGGTKVEIKRTVAAPSVFIFPPSD | |
| | EQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| 13E4_VL3 | DIQMTQSPSSLSASVGDRVTITCRTSENIYNNLAWYQQKP | 329 |
| | GKAPKLLIYAATNLAEGVPSRFSGSGSGTDYTLTISSLQPE | |
| | DFATYYCQHFWGTPLTFGGGTKVEIKRTVAAPSVFIFPPSD | |
| | EQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| 13E4_VL4 | DIQMTQSPSSLSASVGDRVTITCRTSENIYSNLAWYQQKP | 330 |
| | GKAPKLLIYAGTNLADGVPSRFSGSGSGTDYTLTISSLQPE | |
| | DFANYYCQHFWGTPLTFGGGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |

In some aspects, an anti-transferrin receptor antibody described herein has an improved serum half-life compared to a reference anti-transferrin receptor antibody. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference anti-transferrin receptor antibody.

In some aspects, suitable anti-transferrin receptor antibodies as described herein are disclosed in, e.g., U.S. Pat. Nos. 10,913,800, 10,881,743, 11,446,387, 11,555,190, 11,912,779, 10,994,020, 12,071,621, U.S. patent application Ser. No. 18/755,579, U.S. patent application Ser. No. 18/759,724, or U.S. patent application Ser. No. 18/903,935. The content of these US patents and US patent applications are incorporated herein by reference in their entireties.

Other suitable anti-transferrin receptor antibodies are disclosed in US Patent Publication No. 2023/0285586, U.S. Pat. No. 11,771,776, US Patent Publication No. 2023/0144436, US Patent Publication No. 2024/0016952, international patent publication No. WO2023/201332, US Patent Publication No. 2023/0256112, US Patent Publication No. 2023/0113823, US Patent Publication No. 2023/010379, international patent publication No. WO2024/006976, international patent publication No. WO2024/036096, international patent publication No. WO2022/020105, international patent publication No. WO2021/154476, international patent publication No. WO2021/154477, international patent publication No. WO2023/283620, U.S. Pat. Nos. 11,672,872, 11,839,660, 11,969,475, international patent publication No. WO2025/085352, international patent publication No. WO2024/026474, and US Patent Publication No. 2025/235549, each of which is incorporated herein by reference in its entirety.

In some aspects, the number of oligonucleotide molecule conjugated to an antibody forms a ratio. In some instances, the ratio is referred to as a DAR (drug-to-antibody ratio), in which the drug as referred to herein is the oligonucleotide molecule. In some instances, the DAR ratio of the oligonucleotide molecule to antibody is about 1, 2, 3, 4, 5, 6, 7, 8, 9, 10, 11, 12, 13, 14, 15, 16, or greater. In some instances, the DAR ratio of the oligonucleotide molecule to antibody is at least about 1, 2, 3, 4, 5, 6, 7, 8, 9, 10, 11, 12, 13, 14, 15, 16, or greater. In some instances, the DAR ratio of the oligonucleotide molecule to antibody is at most about 1, 2, 3, 4, 5, 6, 7, 8, 9, 10, 11, 12, 13, 14, 15, or 16. In some aspects, the DAR ratio of the oligonucleotide molecule to antibody is about 12. In some aspects, the DAR ratio of the oligonucleotide molecule to antibody is about 8. In some aspects, the DAR ratio of the oligonucleotide molecule to antibody is about 6. In some aspects, the DAR ratio of the oligonucleotide molecule to antibody is about 4. In some aspects, the DAR ratio of the oligonucleotide molecule to antibody is about 2. In some aspects, the DAR ratio of the oligonucleotide molecule to antibody is about 1.

In some aspects, the formulation comprises a plurality of antibody-polynucleic acid conjugates. In some instances, the plurality of antibody-polynucleic acid conjugates in the composition has different DARs. In some instances, at least two of the antibody-polynucleic acid conjugates in the composition have different DARs to each other. In some instances, the DAR is an average DAR (drug-to-antibody ratio), which is an average number of the DARs of the plurality of antibody-polynucleic acid conjugates in the composition. In some instances, the average DAR of the polynucleic acid molecule to antibody is about 1, 2, 3, 4, 5, 6, 7, 8, 9, 10, 11, 12, 13, 14, 15, 16, or greater. In some instances, the average DAR of the polynucleic acid molecule to antibody is approximately 1, 2, 3, 4, 5, 6, 7, 8, 9, 10, 11, 12, 13, 14, 15, 16, or greater. In some instances, the average DAR of the polynucleic acid molecule to antibody is about 1, 2, 3, 4, 5, 6, 7, 8, 9, 10, 11, 12, 13, 14, 15, or 16. In some instances, the average DAR of the polynucleic acid molecule to antibody is in the range of 1.5-2.5, 2.5-3.5, 3.5-4.5, 4.5-5.5, 5.5-6.5, 6.5-7.5, 7.5-8.5, 8.5-9.5, 9.5-10.5, 10.5-11.5, 11.5-12.5, 12.5-13.5, 13.5-14.5, 14.5-15.5, 15.5-16.5, or 16.5-17.5. In some instances, the average DAR of the polynucleic acid molecule to antibody is in the range of 1.5-2.5. In some instances, the average DAR of the polynucleic acid molecule to antibody is in the range of 2.5-3.5. In some instances, the average DAR of the polynucleic acid molecule to antibody is in the range of 3.5-4.5. In some instances, the average DAR of the polynucleic acid molecule to antibody is in the range of 4.5-5.5. In some instances, the average DAR of the polynucleic acid molecule to antibody is in the range of 5.5-6.5. In some instances, the average DAR of the polynucleic acid molecule to antibody is in the range of 6.5-7.5. In some instances, the average DAR of the polynucleic acid molecule to antibody is in the range of 7.0-7.5. In some instances, the average DAR of the polynucleic acid molecule to antibody is in the range of 7.5-8.5. In some instances, the average DAR of the polynucleic acid molecule to antibody is in the range of 8.5-9.5. In some instances, the average DAR of the polynucleic acid molecule to antibody is in the range of 9.5-10.5. In some instances, the average DAR of the polynucleic acid molecule to antibody is in the range of 10.5-11.5. In some instances, the average DAR of the polynucleic acid molecule to antibody is in the range of 11.5-12.5. In some instances, the average DAR of the polynucleic acid molecule to antibody is in the range of 12.5-13.5. In some instances, the average DAR of the polynucleic acid molecule to antibody is in the range of 13.5-14.5. In some instances, the average DAR of the polynucleic acid molecule to antibody is in the range of 14.5-15.5. In some instances, the DAR of the polynucleic acid molecule to antibody is in the range of 15.5-16.5. In some instances, the average DAR of the polynucleic acid molecule to antibody is in the range of 16.5-17.5.

In some aspects, an antibody or antigen binding fragment is further modified using conventional techniques known in the art, for example, by using amino acid deletion, insertion, substitution, addition, and/or by recombination and/or any other modification (e.g., posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor.

In some instances, an antigen binding fragment further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such aspects, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some aspects, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its antigen binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further aspects, the binding is exemplified by the specificity of a “key-lock-principle”. Thus, in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or antigen binding fragment or a reduced off-target effect. For example, the antibody or antigen binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Antibody-Cargo Conjugates

### Cargo

In some aspects, the antibody or antigen binding fragment described herein is conjugated to a cargo. In some instances, the cargo molecule that is delivered to the target by the antibody or antigen binding fragment conjugated therewith. In some instances, the cargo is a drug or a drug moiety. In some cases, the cargo comprises small molecules, amino acids, peptides, polypeptides, proteins, toxins, hormones, lipids, sugars, carbohydrates, polymers such as polyethylene glycol and polypropylene glycol, as well as analogs or derivatives of all of these classes of substances. Additional examples of cargo also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides.

In some aspects, the cargo is a cytotoxic agent. Examples of the cytotoxic agent include, e.g., maytansinoids, dalicheamicins, duocarmycin, and doxorubicin. Further examples include, but are not limited to monomethyl auristatin E (MMAE), monomethyl auristatin F (MMAF), calicheamicin, SN-38 and exatecan.

In some aspects, the cargo is a labeling agent (e.g., fluorescent molecule, radiolabeling molecule, etc.). In some aspects, the moiety is a chelating agent.

### Lipophilic Moiety

In some embodiments, the antibody-oligonucleotide conjugates described herein comprises a lipophilic moiety. In some embodiments, the lipophilic moiety is a linear or branched alky group. In some embodiments, the linear alkyl group includes, but not limited to, saturated or unsaturated C4 to C30 alkyl chains. In some embodiments, the linear alkyl group comprises unsubstituted or substituted C4, C6, C8, C10, C12, C14, C16, C18, C20, C22, C24, C26, C28 and C30 hydrocarbon chains. In some embodiments, the linear alkyl group comprises unsubstituted or substituted C14-C22 hydrocarbon chains. In some embodiments, the linear alkyl group comprises unsubstituted or substituted C14, C16, C18, C20, C22 hydrocarbon chains.

In some embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprise a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₂₂ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₂₂ alkyl or alkenyl). In one embodiment, the lipophilic moiety contains a saturated or unsaturated C₁₆ hydrocarbon chain (e.g., a linear C₁₆ alkyl or alkenyl). In one embodiment, the lipophilic moiety contains a saturated or unsaturated C₂₂ hydrocarbon chain (e.g., a linear C₂₂ alkyl or alkenyl). In one embodiment, the lipophilic moiety is attached to the 2′-OH of a nucleotide. In one embodiment, the lipophilic moiety is attached to the 3′-OH of a nucleotide. In one embodiment, the lipophilic moiety is a lipid having an unsaturated C₁₆ hydrocarbon chain (C16). In one embodiment, the lipophilic moiety is a lipid having an unsaturated C₂₂ hydrocarbon chain (C22).

In some embodiments, the lipophilic moiety is covalently conjugated to a polynucleotide molecule. In some embodiments, the lipophilic moiety is conjugated to a single-stranded polynucleotide molecule. In some embodiments, the lipophilic moiety is conjugated to a double-stranded polynucleotide molecule. In some embodiments, the lipophilic moiety is conjugated to an siRNA, ASO, PMO, or the like. In some embodiments, the lipophilic moiety is conjugated to an siRNA comprising a guide strand and a passenger strand. In some embodiments, the lipophilic moiety is conjugated to a guide strand of the siRNA. In some embodiments, the lipophilic moiety is conjugated to the 5′ end of the guide strand. In some embodiments, the lipophilic moiety is conjugated to the 3′ end of the guide strand. In some embodiments, the lipophilic moiety is conjugated to a passenger strand of the siRNA. In some embodiments, the lipophilic moiety is conjugated to the 5′ end of the passenger strand. In some embodiments, the lipophilic moiety is conjugated to the 3′ end of the passenger strand.

In some embodiments, the lipophilic moiety is conjugated to an internal position of the guide strand. In some embodiments, the lipophilic moiety is conjugated to a nucleotide of the guide strand at a position 1, 2, 3, 4, 5, 6, 7, 8, 9, 10, 11, 12, 13, 14, 15, 16, 17, 18, 19, 20 or 21 from the 5′ end. In some embodiments, the lipophilic moiety is conjugated to a nucleotide within the seed region of the guide strand (e.g., at positions 2-8 from the 5′ end). In some embodiments, the lipophilic moiety is conjugated to a nucleotide of the guide strand outside the seed region. In some embodiments, the lipophilic moiety is conjugated to a nucleotide of the guide strand at one of positions 16-21 from the 5′ end. In some embodiments, the lipophilic moiety is conjugated to a nucleotide of the guide strand at one of positions 7-10 from the 5′ end. In some embodiments, the lipophilic moiety is conjugated to a nucleotide of the guide strand at position 2, 3, 6, 9, 12, 17, or 19 from the 5′ end. In some embodiments, the lipophilic moiety is conjugated to a nucleotide of the guide strand at position 2, 3 or 6 of the guide strand.

In some embodiments, the lipophilic moiety is conjugated to an internal position of the passenger strand. As used herein the position of the nucleotide is counted from the 5′ end. Thus, for example, position 2 nucleotide of the passenger strand is a second nucleotide from the 5′ end of the passenger strand. In some embodiments, the lipophilic moiety is conjugated to a nucleotide at position 1, 2, 3, 4, 5, 6, 7, 8, 9, 10, 11, 12, 13, 14, 15, 16, 17, 18, or 19 of the passenger strand. In some embodiments, the lipophilic moiety is conjugated to a nucleotide at position 2, 3, 6, 9, 12, 17, or 19 of the passenger strand. In some embodiments, the lipophilic moiety is conjugated to a nucleotide at position selected from 2-8 of the passenger strand. In some embodiments, the lipophilic moiety is conjugated to a nucleotide at position 2, 3 or 6 of the passenger strand.

In some embodiments, the polynucleotide conjugate comprises one or more lipophilic moieties. In some embodiments, the one or more lipophilic moieties are same type of lipophilic moiety. For example, both lipophilic moieties are C16 saturated hydrocarbon chains. In some embodiments, the one or more lipophilic moieties are different types of lipophilic moiety. For example, one lipophilic moiety is a C16 saturated hydrocarbon chain, and another lipophilic moiety is C22 unsaturated hydrocarbon chain. In some embodiments, the polynucleotide conjugate comprises one or more lipophilic moieties that are conjugated to the polynucleotide conjugate via a single linker. In some embodiments, the polynucleotide conjugate comprises one or more lipophilic moieties that are conjugated to the polynucleotide conjugate via multiple linkers.

In some embodiments, the one or more lipophilic moieties are conjugated to one or more nucleotides at terminal and/or internal positions in the guide strand of siRNA. In some embodiments, the one or more lipophilic moieties are conjugated to one or more nucleotides at terminal and/or internal positions in the passenger strand of siRNA. In some embodiments, the one or more lipophilic moieties are conjugated to one or more nucleotides of the guide strand at positions selected from 1, 2, 3, 4, 5, 6, 7, 8, 9, 10, 11, 12, 13, 14, 15, 16, or 17 from the 5′ end. In some embodiments, the one or more lipophilic moieties are conjugated to one or more nucleotides of the passenger strand at positions selected from 1, 2, 3, 4, 5, 6, 7, 8, 9, 10, 11, 12, 13, 14, 15, 16, 17, 18 or 19 from the 5′ end. In some embodiments, the one or more lipophilic moieties are conjugated to one or more nucleotides of the guide strand at positions selected from 2, 3, 6, 9, 12, 17, or 19 from the 5′ end. In some embodiments, the one or more lipophilic moieties are conjugated to one or more nucleotides of the passenger strand at positions selected from 2, 3, 6, 9, 12, 17, or 19 from the 5′ end. In some embodiments, the one or more lipophilic moieties are conjugated to one or more nucleotides of the guide strand at positions selected from 3 or 6 from the 5′ end. In some embodiments, the one or more lipophilic moieties are conjugated to one or more nucleotides of the passenger strand at positions selected from 2, 3 or 6 from the 5′ end. In some embodiments, the one or more lipophilic moieties are conjugated to one or more nucleotides at terminal and/or internal positions in a single-stranded polynucleotide molecule, such as ASO or PMO.

In some embodiments, the lipophilic moiety is conjugated to a sugar ring of the nucleotide. In some embodiments, the lipophilic moiety is conjugated to a sugar moiety of a nucleotide of the polynucleotide molecule at the 2′-carbon or a modification made thereof. In some embodiments, the lipophilic moiety is conjugated to a sugar moiety of a nucleotide of the polynucleotide molecule at the 3′-carbon or a modification made thereof.

In some embodiments, the lipophilic moiety is conjugated to at least one 2′ modified nucleotide. In some embodiments, the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the at least one 2′ modified nucleotide comprises locked nucleic acid (LNA) or ethylene nucleic acid (ENA). In some embodiments, one or more lipophilic moieties are conjugated to at least one 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the lipophilic moiety is covalently to 2′-MOE modified nucleotide.

### Pharmaceutical Formulation

In some aspects, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, lyophilized forms, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin or dextrin or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some aspects, the pharmaceutical formulation comprises a delivery vector, e.g., a recombinant vector, the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

In some aspects, the pharmaceutical formulation includes a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulation further includes pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulation further includes diluent which are used to stabilize compounds because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulation includes disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulation includes filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Definition

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

Use of absolute or sequential terms, for example, “will,” “will not,” “shall,” “shall not,” “must,” “must not,” “first,” “initially.” “next.” “subsequently,” “before.” “after.” “lastly,” and “finally,” are not meant to limit scope of the present embodiments disclosed herein but as exemplary.

Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The immunoconjugates described herein comprise antigen binding regions. These antigen binding regions can be derived from an “antibody.” The term “antibody” herein is used in the broadest sense and includes monoclonal antibodies, and includes intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. An antibody fragment retains at least some of the binding specificity of the parental antibody. Typically, an antibody fragment retains at least 10% of the parental binding activity. Preferably, an antibody fragment retains at least 20%, 50%, 70%, 80%, 90%, 95% or 100% or more of the parental antibody's binding affinity for the target. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each variable domain (CDR-H1, CDR-H2, CDR-H3) and three CDRs in each light chain variable region (CDR-L1, CDR-L2, CDR-L3). “Framework regions” and “FR” are known in the art to refer to the non-CDR portions of the VHH variable regions of the heavy and light chains. In general, there are four FRs in each full-length variable domain (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The VHH variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

The antibodies of the immunoconjugates described herein may comprise an Fc domain. An Fc domain generally encompasses and/or refers to a C-terminal region of an immunoglobulin heavy chain that contains at least a portion of the constant region, such as an immunoglobulin CH2 and CH3 domain. Fc fragments generally exclude a CH1 domain and in some instances an immunoglobulin hinge region. The term includes native sequence Fc regions and variant Fc regions. For example, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. In certain embodiments, the Fc region include IgG and sub-classes thereof (e.g., IgG1 and IgG4), IgM, IgE, IgA, and/or IgD heavy chain constant regions and/or heavy chain constant regions derived from IgG and sub-classes thereof (e.g., IgG1 and IgG4), IgM, IgE, IgA, and IgD. Generally, the antibodies described herein have the format variable domain—hinge—Fc domain, wherein the hinge sequence is included as part of the Fc domain sequence.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The “specificity” of an amino acid sequence, in particular an antibody fragment, such as a VHH, or functional fragments thereof as disclosed herein can be determined based on affinity and/or avidity and is generally derived from the CDR regions without contribution from other parts of the antibody such as constant regions. In some embodiments, the amino acid sequences as disclosed herein will bind to a target protein of interest with a dissociation constant (KD) of less than about 1 micromolar (1 μM). A KD value greater than about 1 millimolar is indicates non-binding or non-specific binding. Binding affinities may be determined by means or methods known to the person skilled in the art, for example ELISA methods, isothermal titration calorimetry, surface plasmon resonance, fluorescence-activated cell sorting analysis, and the like.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). The antibody, or antigen binding polypeptide is capable of binding antigen with sufficient affinity such that the antibody is useful as a diagnostic and/or therapeutic agent in targeting that antigen.

As used herein, the term “linker” refers to a moiety that attaches one molecule to another molecule (e.g., attaches an oligonucleotide to an antibody fragment such as a VH or functional fragment thereof). The linker may be a chemical linker, a nucleotide linker, a peptide linker, or any combination thereof.

As used herein, the terms “polynucleotide,” “polynucleotide molecule,” “polynucleic acid molecule” and “oligonucleotide,” are used interchangeably to refer to a nucleic acid molecule comprising a plurality of nucleosides linked via internucleotide linkages.

As used herein, the term “sense strand” can be interchangeably used with the term “passenger strand,” and the term “antisense strand” can be interchangeably used with the term “guide strand.”

As used herein, the term “chelator” or “chelating moiety” refers to a chemical compound to which a metal, preferably a radiometal, can be chelated via coordinate bonding.

An “isolated” antibody or immunoconjugate or radio immunoconjugate is one which has been separated from a component of its natural environment or artificial production. In some embodiments, an antibody is purified as determined by, for example, electrophoretic (e.g., SDS-PAGE, isoelectric focusing (IEF), capillary electrophoresis) or chromatographic (e.g., ion exchange or reverse phase HPLC). Routine methods for assessment of antibody purity in a composition are known to the skilled worker, see e.g., Flatman et al., J. Chromatogr. B 848:79-87 (2007). In particular, unwanted components (contaminants) to be purified away from are such components that would interfere with desired uses for the antibody, such as, e.g., a therapeutic use, and may include, inter alia, bacterial factors, enzymes, hormones, and other proteinaceous or non-proteinaceous solutes.

The “transition metal”, refers to the elements found in the central block (group 3-12) of the periodic table. These elements are known for their ability to form stable compounds, exhibit various oxidation states, and serve as catalysts in chemical reactions.

The terms “molar equivalents (meqs)” and “equivalents” are used interchangeably to refer to the ratio of moles of one substance relative to the moles of another substance in a reaction. In antibody-drug or antibody-oligonucleotide conjugation, “molar equivalents” refers to the ratio of the moles of reagent (reducing agent, linker, or transition metal) to the moles of antibody used.

As used herein, the term “comprising” and its derivatives are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

As used herein, the term “consisting of” and its derivatives are intended to be closed ended terms that specify the presence of stated features, elements, components, groups, integers, and/or steps, and also exclude the presence of other unstated features, elements, components, groups, integers and/or steps.

As used herein, the term “consisting essentially of” is intended to specify the presence of the stated features, elements, components, groups, integers, and/or steps as well as those that do not materially affect the basic and novel characteristic(s) of features, elements, components, groups, integers, and/or steps.

### EMBODIMENTS

Embodiment 1. A method of preparing a binding moiety-oligonucleotide conjugate comprising the steps of
a) contacting a binding moiety in an acidic buffer with a reducing agent and a transition metal or its salts thereof to generate a first mixture;
b) contacting the first mixture with an oxidizing agent to generate a second mixture;
c) adding an oligonucleotide molecule conjugated with a linker to the second mixture to generate a binding moiety-oligonucleotide conjugate comprising the binding moiety conjugated to the oligonucleotide molecule via the linker; and
d) isolating the binding moiety-oligonucleotide conjugate, thereby preparing the binding moiety-oligonucleotide conjugate.

Embodiment 2. The method of embodiment 1, wherein the acidic buffer has a pH of less than 7.0.

Embodiment 3. The method of embodiment 1 or 2, wherein the acidic buffer has a pH of about 6.5.

Embodiment 4. The method of any one of embodiments 1-3, wherein the acidic buffer comprises a citrate/sucrose buffer or a histidine buffer.

Embodiment 5. The method of any one of embodiments 1-4, wherein the step a) comprises about 10 molar equivalents of the reducing agent.

Embodiment 6. The method of any one of embodiments 1-5, wherein the reducing agent comprises tris(2-carboxyethyl)phosphine (TCEP) or dithiothreitol (DTT).

Embodiment 7. The method of any one of embodiments 1-6, wherein the step a) comprises about 1-10 molar equivalents of the transition metal or its salts thereof.

Embodiment 8. The method of any one of embodiments 1-7, wherein the transition metal or its salts thereof comprises zinc (Zn) or its salts thereof, silver (Ag) or its salts thereof, or gold (Au) or its salts thereof.

Embodiment 9. The method of any one of embodiments 1-8, wherein the transition metal or its salts thereof comprises zinc citrate, zinc trifluoromethane sulfonate, or zinc acetylacetonate.

Embodiment 10. The method of any one of embodiments 1-9, wherein the step a) further comprises purifying the first mixture using a phosphate buffer, HEPES buffer, a MES buffer.

Embodiment 11. The method of any one of embodiments 1-10, wherein (i) the binding moiety contacts the reducing agent and the transition metal or its salts thereof concurrently, or (ii) the binding moiety contacts the reducing agent prior to contacting the transition metal or its salts thereof.

Embodiment 12. The method of any one of embodiments 1-11, wherein the step b) comprises less than about 15, 14, 13, 12 or 11 molar equivalents of the oxidizing agent.

Embodiment 13. The method of any one of embodiments 1-12, wherein the step b) comprises about 1-10 molar equivalents of the oxidizing agent.

Embodiment 14. The method of any one of embodiments 1-13, wherein the oxidizing agent comprises dehydroascorbic acid (DHAA).

Embodiment 15. The method of any one of embodiments 1-14, wherein the step c) comprises about 1-3 molar equivalents of the oligonucleotide molecule conjugated with the linker (oligonucleotide-linker).

Embodiment 16. The method of any one of embodiments 1-15, wherein the step c) comprises about 1, 1.05, 1.1, 1.5, 2, 2.15, 2.25, 2.5 or 3 molar equivalents of the oligonucleotide-linker.

Embodiment 17. The method of any one of embodiments 1-16, wherein the step d) comprises isolating the binding moiety-oligonucleotide conjugate via chromatography or filtration.

Embodiment 18. The method of embodiment 17, wherein the chromatography comprises strong anion chromatography (SAX).

Embodiment 19. The method of embodiment 17, wherein the filtration comprises tangential flow filtration (TFF).

Embodiment 20. The method of any one of embodiments 1-19, wherein the method further comprises e) adding an additional oligonucleotide-linker to the isolated binding moiety-oligonucleotide conjugate, wherein the additional oligonucleotide-linker comprises an oligonucleotide molecule that is different from the oligonucleotide molecule in step c), thereby obtaining the binding moiety-oligonucleotide conjugate having different oligonucleotides.

Embodiment 21. The method of any one of embodiments 1-20, wherein the binding moiety is an antibody or antigen binding fragment thereof.

Embodiment 22. The method of any one of embodiments 1-20, wherein the binding moiety comprises a lipid.

Embodiment 23. The method of embodiment 22, wherein the lipid comprises a C16, C20, or C22 alkyl chain.

Embodiment 24. The method of any one of embodiments 1-23, wherein the oligonucleotide comprises an siRNA, an ASO, or a PMO.

Embodiment 25. The method of embodiment 24, wherein the siRNA comprises a guide strand and a passenger strand, wherein the passenger strand is modified with a lipid moiety.

Embodiment 26. The method of embodiment 25, wherein the lipid moiety comprises a C16, C20, or C22 alkyl chain.

Embodiment 27. The method of embodiment 25 or 26, wherein the lipid moiety is conjugated to 3rd or 6th nucleotide from the 5′ end of the passenger strand.

Embodiment 28. The method of any one of embodiments 1-27, wherein the linker comprises a maleimide group.

Embodiment 29. The method of any one of embodiments 1-28, wherein the linker comprises a MCC linker, a MC linker, a MBS linker, or a bismaleimide (Bismal) linker.

Embodiment 30. The method of any one of embodiments 1-29, wherein a yield of the isolated binding moiety-oligonucleotide conjugate having the DAR of about 1 or about 2 is greater than 50%, 60%, 70%, 80% or more.

Embodiment 31. An antibody-Zn complex comprising a zinc and an antibody or antigen binding fragment thereof wherein the zinc is coordinated with a first histidine residue and a first cysteine residue on a first heavy chain of the antibody or antigen binding fragment thereof, a second histidine residue on a second heavy chain of the antibody or antigen binding fragment thereof, and a second cysteine residue on a first light chain of the antibody or antigen binding fragment thereof.

Embodiment 32. The antibody-Zn complex of embodiment 31, wherein the antibody comprises an IgG1 framework.

Embodiment 33. The antibody-Zn complex of embodiment 31 or 32, wherein the antibody is a human IgG1 antibody or a humanized IgG1 antibody.

Embodiment 34. A method of preparing an antibody-oligonucleotide conjugate or an antigen binding fragment-oligonucleotide conjugate comprising steps of:
a) contacting an antibody or antigen binding fragment thereof with a reducing agent;
b) reacting the antibody or antigen binding fragment thereof with a transition metal or its salts thereof;
c) contacting the antibody or antigen binding fragment thereof from step b) with an oxidizing agent;
d) quenching the oxidizing agent using a mineral or organic buffer;
e) chelating the transition metal using a chelating agent; and
f) contacting an oligonucleotide molecule conjugated with a linker (oligonucleotide-linker) to the antibody or antigen binding fragment thereof, thereby generating an antibody-oligonucleotide conjugate or antigen binding fragment-oligonucleotide conjugate.

Embodiment 35. The method of embodiment 34, wherein the organic or mineral buffer comprises a borate buffer.

Embodiment 36. The method of embodiment 34 or 35, wherein the step d) is performed for about 1-2 hours and the step e) is performed for about less than 1 hour.

Embodiment 37. The method of any one of embodiments 34-36, wherein the step d) is performed for about 1 hour and the step e) is performed for about 15-30 minutes.

Embodiment 38. The method of any one of embodiments 34-37, wherein the method further comprises purifying the antibody or antigen binding fragment thereof obtained from the step (a).

Embodiment 39. The method of any one of embodiments 34-38, wherein the method further comprises capping the antibody or antigen binding fragment thereof in the antibody-oligonucleotide conjugate or the antigen binding fragment-oligonucleotide conjugate generated from the step f) using an alkylating reagent.

Embodiment 40. The method of any one of embodiments 34-39, wherein the alkylating agent comprises N-ethylmaleimide (NEM).

Embodiment 41. The method of any one of embodiments 34-40, wherein the step g) comprises about 1-1.5 molar equivalents of the alkylating agent.

Embodiment 42. The method of any one of embodiments 34-41, wherein the chelating agent comprises ethylenediaminetetraacetic acid (EDTA).

Embodiment 43. The method of any one of embodiments 34-42, wherein the method further comprises isolating the antibody-oligonucleotide conjugate or antigen binding fragment-oligonucleotide conjugate via chromatography or filtration.

Embodiment 44. The method of embodiment 43, wherein the chromatography comprises strong anion chromatography (SAX).

Embodiment 45. The method of embodiment 43, wherein the filtration comprises tangential flow filtration (TFF).

### EXAMPLES

These examples are provided for illustrative purposes only and not to limit the scope of the claims provided herein.

### Example 1: Method of Preparing an Anti-TfR1 Monoclonal Antibody-siRNA Conjugate

### Materials

Antibody: The antibody is an IgG1 anti-human TfR1 monoclonal antibody. The antibody is described in U.S. Pat. No. 10,913,800, the content of which are hereby incorporated by reference herein in its entirety.

siRNA-linker: The siRNA is a double-stranded RNA with a guide and complementary passenger strands. The double-stranded RNA siRNA with a guide and complementary passenger strands are described in U.S. Pat. Nos. 10,881,743, 11,446,387, 11,555,190, 11,912,779, U.S. patent application Ser. No. 18/755,579, and U.S. patent application Ser. No. 18/759,724, the contents of which are hereby incorporated by reference in their entireties. The strands were assembled on solid phase using standard phosphoramidite chemistry and purified by HPLC. The base, sugar and phosphate modifications were used to optimize the potency and stability of the duplex and reduce immunogenicity. Purified single strands were duplexed to get the double-stranded siRNA described above. The MCC maleimide linker or bismal linker is located on 5′ end of the passenger strand.

### Methods

### Strong Anion Exchange (SAX) Chromatography Method for Purification

MPA: 20 mM phosphate buffer, pH 7.2
MPB: 20 mM Phosphate buffer, 1.5 M NaCl, pH 7.2
Detection: UV absorption 280 nm
Column: Tosoh Bioscience, TSKGel SuperQ-5PW, 21.5 mm ID×15 cm, 13 um
Flow rate=8.0 ml/min

### Gradient

| % B | Column Volumes |
|---|---|
| 10 | 0 |
| 20 | 1 |
| 40 | 20 |
| 0 | 1 |

### Strong Anion Exchange (SAX) Method for Analyzing Conjugates

Drug to antibody ratio (DAR) of the antibody-siRNA conjugate was determined by strong anion-exchange chromatography (SAX-HPLC) using the fluorescence detector with an excitation wavelength of 280 nm and emission of 345 nm. Different species in the bioconjugate sample were separated based on their different affinity to the anion exchanger. Relative % area of the unreacted mAb (DAR0) and conjugated antibody species (DART, DAR2) was determined. Separation was achieved using a Thermo ProPAC SAX column and gradient elution with tris buffer and sodium chloride mobile phase.
MPA: 10 mM Tris pH 7.2 20% Ethanol
MPB: 10 mM Tris, 1.5M NaCl pH 7.2 20% Ethanol
FLD: Ex: 280 Em: 345
Column: Thermo Scientific, ProPac™ SAX-10, Bio LC™, 4×250 mm
Flow rate=0.92 ml/min

### Gradient:

| T (min) | % B |
|---|---|
| 0 | 16 |
| 2 | 40 |
| 4 | 53.5 |
| 4.5 | 16 |
| 5.5 | 16 |

Method of Preparing Antibody-siRNA Conjugates without Using Zinc (Zn)

To demonstrate the improved qualities of the present methods (including improved yields), we herein describe the method of preparing antibody-siRNA conjugates without the use of a transition metal such as zinc.

Here, the antibody-siRNA conjugate is an anti-human TfR1 monoclonal antibody conjugated to an siRNA via a MCC linker (hTfR1.mAb-MCC-siRNA conjugate), and was prepared with a method without using Zn.

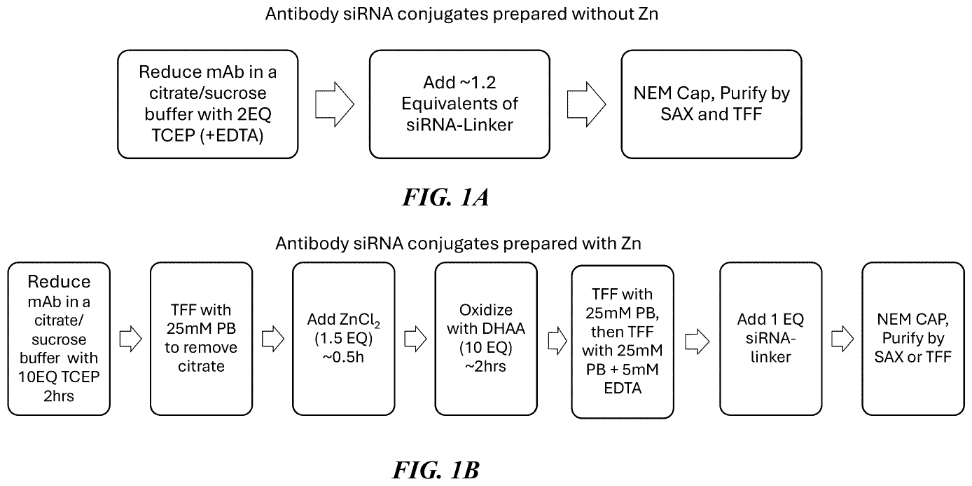

The steps for the method for the preparation of antibody-siRNA conjugates without using Zn are shown in FIG. 1A.

In this example, antibody-siRNA conjugates were prepared using a standard cysteine conjugation method without Zn. An anti-human TfR1 monoclonal antibody mixture solution was prepared with anti-human TfR1 monoclonal antibody at 20 g/L (0.136 mM) in a buffer solution comprising 50 mM citrate and 300 mM sucrose, and 2 mM EDTA, and the pH was adjusted to pH 6.5. The anti-human TfR1 monoclonal antibody mixture solution was then incubated with 2 molar equivalents (meq) of TCEP (0.272 mM) at room temperature for two hours to reduce the antibody. To the reduced antibody solution, 1 meq of siRNA having MCC linker at the 5′ end of the passenger strand was added, and the reaction mixture was incubated at room temperature for 30 minutes. The reaction mixture was further mixed with 10 meq of N-ethylmaleimide (NEM) and incubated at room temperature for 30 minutes. The reaction mixture was analyzed by strong anion exchange (SAX) chromatography to quantify the siRNA to antibody ratio (DAR) followed by purification by SAX chromatography to isolate antibody-siRNA conjugate with DAR of approximately 1 (DAR1) and DAR of approximately 2 (DAR2) and tangential flow filtration with a 50 kDa MWCO membrane to concentrate the antibody-siRNA conjugate in citrate buffer. The yield of DAR1 antibody-siRNA conjugates prepared without Zn was approximately 55% (see Table 8).

TABLE 8

| | Antibody-siRNA | Antibody-siRNA |
|---|---|---|
| | Conjugate without Zn | conjugate with Zn |
| DAR 0 (% AUC) | 31 | 10 |
| DAR 1 (% AUC) | 56 | 83 |
| DAR 2 (% AUC) | 13 | 7 |

Method of Preparing Antibody-siRNA Conjugates with Zinc (Zn)

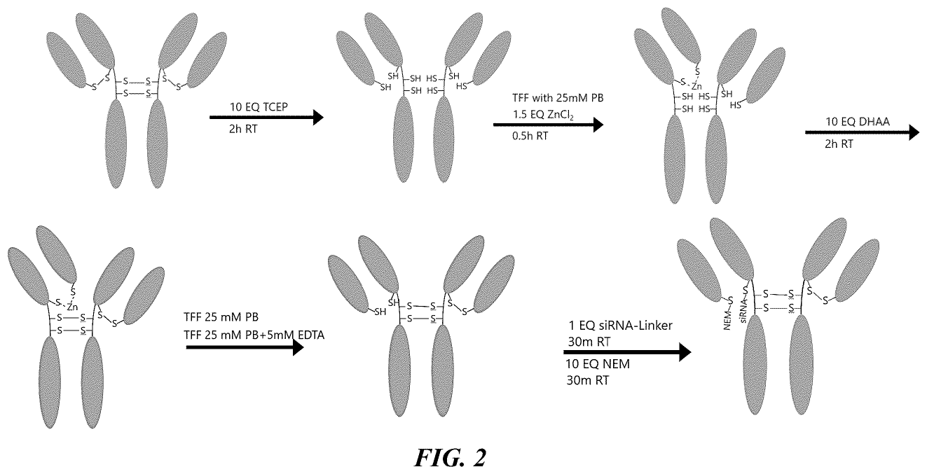

In this example, the anti-TfR1 monoclonal antibody-siRNA conjugate is an anti-human TfR1 monoclonal antibody conjugated to an siRNA via a MCC linker (hTfR1.mAb-MCC-siRNA conjugate) and was prepared using Zn. The steps for the method for the preparation of antibody-siRNA conjugates using Zn are shown in FIG. 1B and FIG. 2.

In this example, antibody-siRNA conjugates were prepared using a standard cysteine conjugation method using Zn. Antibody-siRNA conjugates were prepared by adding 10 molar equivalents (meq) of Tris(2-carboxyethyl)phosphine (TCEP) (1.36 mM) to the solution containing human IgG1 monoclonal antibody targeting hTfR1 (hTfR1.mAb) at 20 g/L (0.136 mM) in a buffer solution comprising 50 mM citrate and 300 mM sucrose at pH 6.5 and incubated at room temperature for 2 hours. The reaction mixture was diafiltered with a 50 kDa MWCO membrane and 8 diavolumes of 25 mM phosphate buffer at pH 7.5, bringing the hTfR1.mAb concentration to 10 mg/ml (0.0679 mM). 1.5 meq of ZnCl₂ (0.1018 mM) was added to the reaction mixture and incubated at room temperature for 30 minutes followed by 10 meq of dehydroascorbic acid (DHAA) (0.679 mM) incubated at room temperature for 2 hours. The reaction mixture was diafiltered with a 50 kDa MWCO membrane and 8 diavolumes of 25 mM phosphate buffer, pH 7.5, followed by diafiltration with 25 mM phosphate buffer with 5 mM ethylenediaminetetraacetic acid (EDTA) bringing the hTfR1.mAb concentration to 10 g/L (0.0679 mM). Then 1 meq of siRNA with a maleimidomethyl cyclohexane-1 carboxylate (MCC) linker at the 5′ end of the passenger strand (0.0679 mM) was added to the mixture. The reaction mixture was incubated at room temperature for 30 minutes, followed by the addition of 10 meq of N-ethylmaleimide (NEM) (0.679 mM) and incubation at room temperature for 30 minutes. The reaction mixture was analyzed by strong anion exchange (SAX) chromatography to quantify the siRNA to antibody ratio (DAR). The reaction was either purified by SAX chromatography or tangential flow filtration (TFF) with a 50 kDa MWCO membrane to isolate antibody-siRNA conjugate with DAR1 and DAR2 and concentrate the antibody-siRNA conjugate in a buffer (e.g. citrate buffer). The yield of the DAR1 antibody-siRNA conjugates prepared with Zn was above 80% (see Table 8).

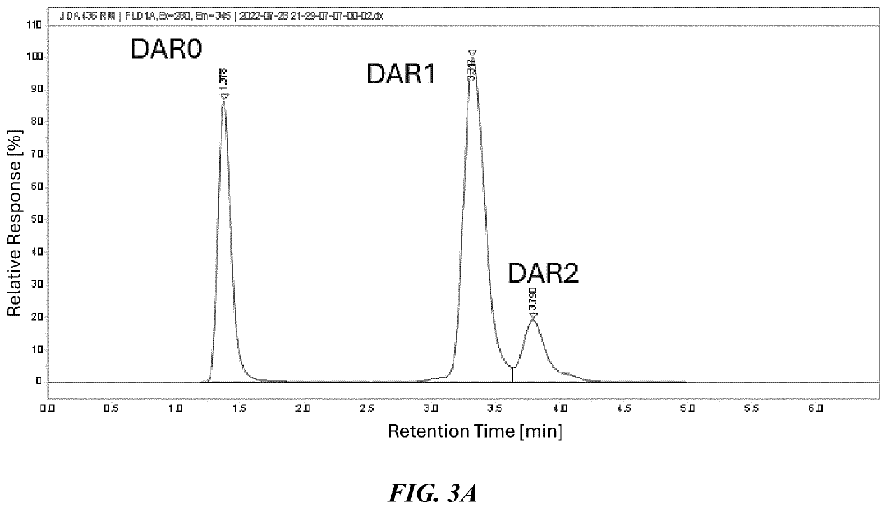

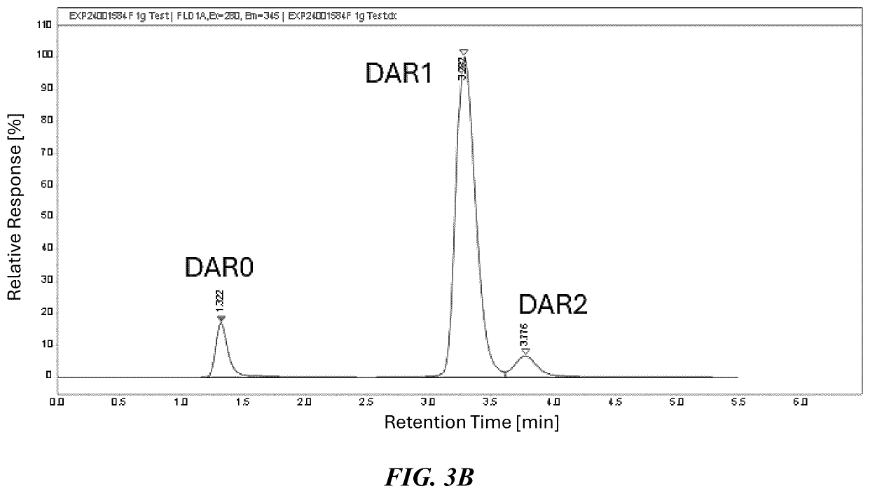

The reaction mixtures for both methods were analyzed by strong anion exchange chromatography (SAX) for DAR distribution of antibody-siRNA conjugates. Analysis of the chromatogram revealed that the method without Zn resulted in a mixture of antibody-siRNA conjugates with about 31% of DAR0, 56% of DAR1, and 13% of DAR2 (see Table 8 and FIG. 3A). When concentrations of siRNA were increased in the method without Zn, percentages of DAR0 antibody-siRNA conjugates decreased and percentages of DAR2 antibody-siRNA conjugates increased in the mixture of antibody-siRNA conjugates. Interestingly, the DAR1 antibody-siRNA conjugate yield by the preparation without Zn did not increase beyond 55%.

Analysis of the chromatogram for method of preparing antibody-siRNA conjugates with Zn revealed in a mixture of an antibody-siRNA conjugates with about 10% of DAR0, 83% of DAR1, and 7% of DAR2 (Table 8, FIG. 3B). The DAR1 antibody-siRNA conjugate yield of more than 80% in the mixture was a significant improvement in the preparation of antibody-siRNA conjugates. The results show that the step of using Zn significantly improves the DAR1 antibody-siRNA conjugates yield in the mixture with a DAR1 yield reaching above 80%.

### Example 2: Analysis of Anti-TfR1 Antibody-siRNA Conjugate Using Reduced and Non-Reduced Capillary Gel Electrophoresis

### Materials

The antibody and siRNA-linker are described in Example 1.

### Methods

### Reducing Capillary Electrophoresis (CE) and Non-Reducing Capillary Electrophoresis (CE)

Capillary electrophoresis (CE-SDS), performed under reducing and non-reducing conditions was used for purity determination and site of siRNA conjugation (HC or LC). The test sample was treated with a solution containing sodium dodecyl sulphate (SDS) and separated by size through a gel filled capillary by applying voltage and passed through a UV detector set at 220 nm. In the reduced method, the test item was reduced using 2-mercaptoethanol to separate the heavy chain and light chain components with and without siRNA. In the non-reduced method, disulfide bonds remained intact, but non-covalent interactions were broken under SDS denaturing conditions, allowing the separation of antibody components with and without siRNA conjugation. Reduced capillary electrophoresis (rCE) and non-reduced capillary electrophoresis (nrCE) were performed using ProteinSimple Maurice Plus. Antibody-siRNA conjugate samples were prepared by diluting to 1 mg/ml with 1× Plus sample buffer followed by the addition of internal standard. 2-mercaptoethanol was added to the reduced samples and iodoacetamide was added to non-reduced samples. Each sample was incubated at 70° C. for 10 min followed by 5 min on ice. Samples were loaded at 4600 volts for 20 seconds, separation of reduced samples was performed at 5750 volts for 25 minutes, non-reduced samples were run at 5750 volts for 35 minutes.

### Analysis of Antibody-siRNA Conjugates Using Non-Reduced Capillary Gel Electrophoresis

### Results

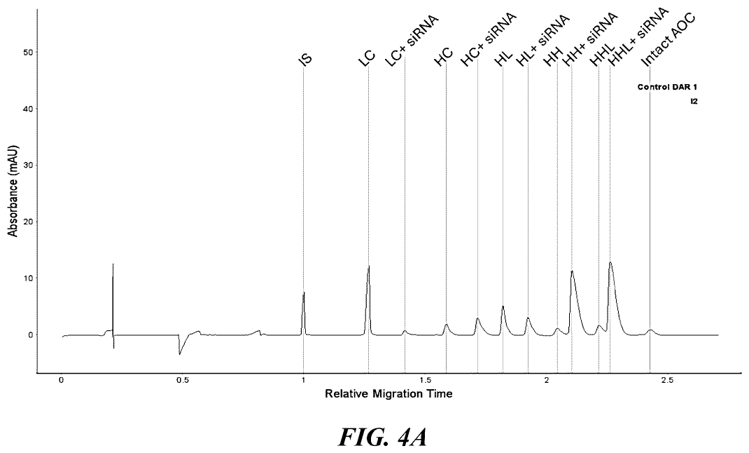

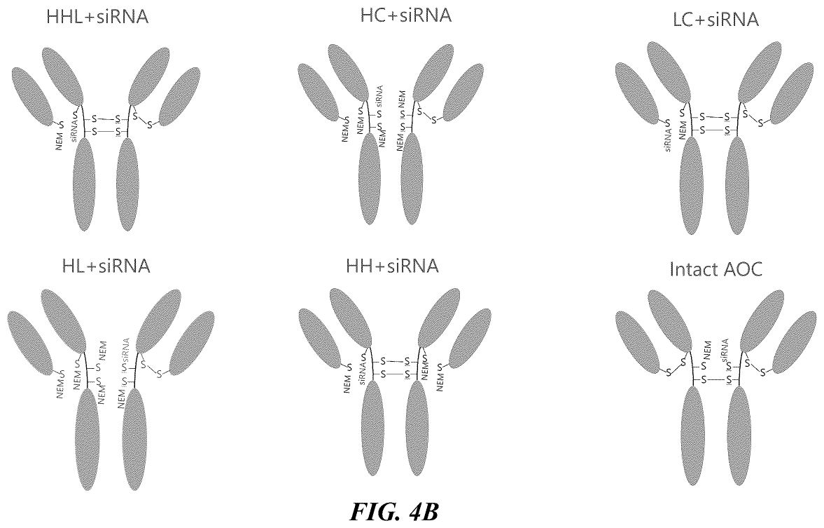

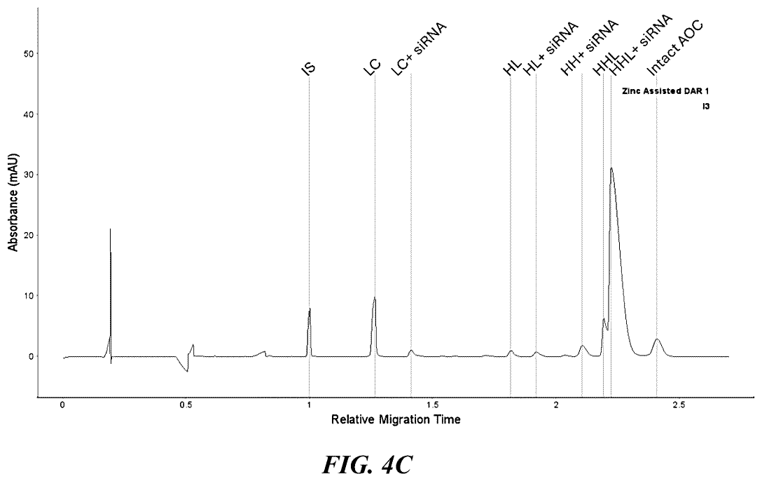

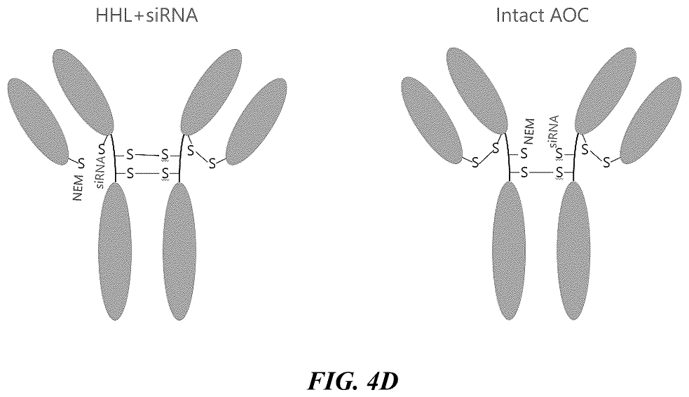

DAR1 antibody-siRNA conjugates prepared with or without Zn were analyzed using non-reduced capillary electrophoresis (CE). The non-reduced CE allows separation based on the size of the antibody-siRNA conjugate fragments. Analysis of the non-reduced CE electrophoresis chromatogram of the method without Zn revealed DAR1 antibody-siRNA conjugates with the majority of the siRNA conjugated to the heavy chain (HC) of the antibody (FIG. 4A). However, a heterogeneous distribution of disulfide formation was observed on the non-reduced CE electrophoresis chromatogram as indicated by the many peaks representing HHL+siRNA, HC+siRNA, LC+siRNA, HL+siRNA, HH+siRNA, and Intact AOC+siRNA (FIG. 4B).

Analysis of the electrophoresis chromatogram for the method with Zn revealed more homogenous profile consisting of mostly HHL+siRNA (FIG. 4C). Analysis of the peaks revealed that the main product was the HHL+siRNA peak, indicating that the disulfides between the two HC were intact, and the disulfide between one HC and one LC were intact, but the disulfide between the second HC and the second LC was open (FIG. 4D). These observations were further supported by the strong signal of LC in the chromatogram.

Overall, antibody-siRNA conjugates prepared using Zn analyzed with non-reduced capillary electrophoresis (CE) resulted in a profile consisting of mostly HHL+siRNA.

### Analysis of Antibody-siRNA Conjugates Using Reduced Capillary Gel Electrophoresis

### Results

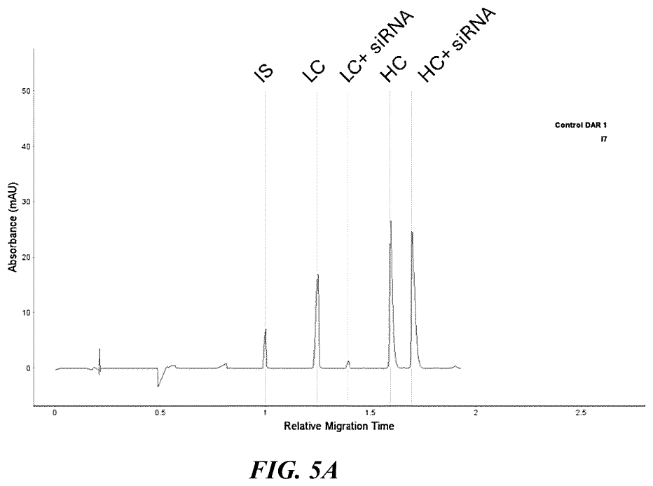

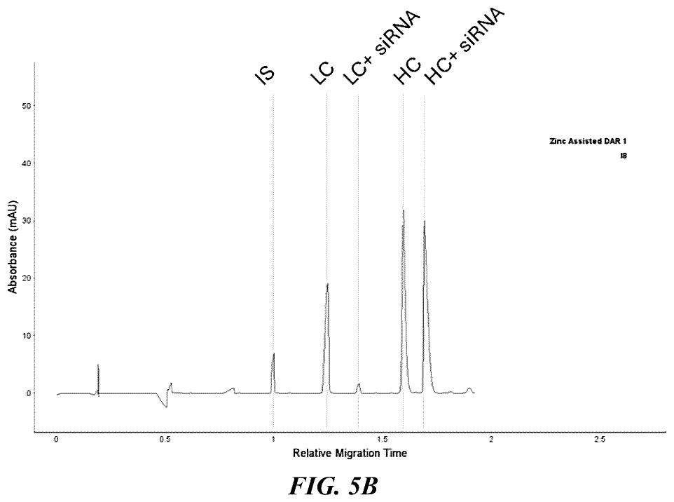

Antibody-siRNA conjugates prepared with both methods were analyzed with the reduced CE under denaturing conditions (with SDS), which open all disulfide bonds and disrupt all non-covalent interactions between antibody fragments and allow separation based on the size of the fragments of the antibody-siRNA conjugate. Analysis of the electrophoresis chromatograms for both methods revealed an identical peak profile (FIGS. 5A-5B). This profile indicates that the majority of the siRNAs was conjugated to the HC by both methods. Interestingly, the method using Zn could result in greater than 90% of the siRNAs conjugated to the heavy chain as shown in FIG. 5B. Overall, the results showed that that the majority of the siRNA is conjugated to the HC by both methods.

### Example 3: Analysis of Anti-TfR1 Antibody-siRNA Conjugates Using Intact Mass Spectrometry

### Materials

The preparation of antibody-siRNA conjugates is described in Example 1.

### Methods

### Native Intact Mass Spectrometry (MS) Method

Intact native mass spectrometry was used for identity confirmation and to determine the number of NEMs conjugated to the antibody-siRNA conjugate. Samples were first deglycosylated with PNGase F then analyzed by SEC-MS using an ammonium acetate mobile phase.
MPA: 50 mM ammonium acetate
Column: MAbPac SEC-1, 300A, 5 μm 4×150 mm (Thermo 075592)
Flow rate=0.3 ml/min
Run Time=10 min
Scan Type: Full
Resolution: 30,000
Scan Range (m/z): 1500-7000
Polarity: Positive

### Reduced Intact Mass Spectrometry (MS) Method

Reduced intact mass spectrometry was used for identity confirmation and to determine the number of NEMs conjugated to the heavy chains and light chains of the antibody. Samples were first deglycosylated with PNGase F and reduced with dithiothreitol (DTT), then analyzed by LCMS.
MPA: 0.05% Trifluoroacetic Acid in Water
MPB: 0.05% Trifluoroacetic Acid in Acetonitrile
Column: BioResolve RP mAb Polyphenyl 450 Å, 2.1×100 mm, 2.7 um (Waters 186008945)
Flow rate=0.5 ml/min

### Gradient:

| Time | | |
|---|---|---|
| (min) | % A | % B |
| 0 | 75 | 25 |
| 0.3 | 75 | 25 |
| 13 | 40 | 60 |
| 13.5 | 10 | 90 |
| 15 | 10 | 90 |
| 15.1 | 75 | 25 |
| 23 | 75 | 25 |

Scan Type: Full
Resolution: 15,000
Scan Range (m/z): 300-4500
Polarity: Positive

### Analysis of Anti-TfR1 Antibody-siRNA Conjugates Using Intact Mass Spectrometry

### Results

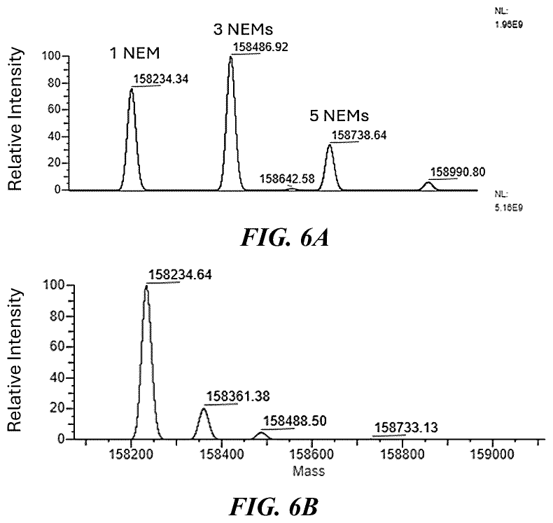

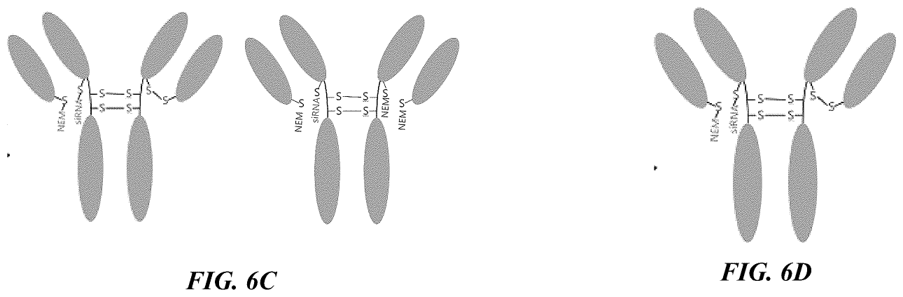

Antibody-siRNA conjugates prepared without Zn or with Zn were analyzed for N-ethylmaleimide (NEM) capping distribution of the conjugates using intact mass spectrometry. As shown in FIG. 6A, analysis of the mass chromatogram of the conjugates prepared without Zn revealed 4 different peaks corresponding to the antibody+siRNA+1, 3, 5, or 7 NEM (FIG. 6A and FIG. 6C). These data indicate that the N-ethyl maleimide (NEM) reacted with any cysteine residues having thiol groups available on the antibody after conjugation with the siRNA-linker. The data is consistent with the observations made with the non-reducing CE that the method without Zn showed a heterogeneous mixture of cysteine residues with open disulfides that react with NEM.

However, analysis of the chromatogram of the conjugates prepared with Zn revealed one main peak corresponding to the antibody+siRNA+1NEM (MW=158234.64) (FIG. 6B and FIG. 6D). The data is consistent with the results obtained with the analysis of the non-reducing CE electrophoresis chromatogram indicating that HHL+siRNA was the main product. Since only one disulfide bond open, one of the thiol group of a cysteine residue reacted with the siRNA-linker and the other cysteine with the thiol group reacted with the NEM. The non-reduced CE-SDS electrophoresis chromatogram showing free LC confirms that the NEM was capped on the LC and that the siRNA was conjugated to the HC.

Overall, the analysis showed that antibody+siRNA+1NEM is the main product when antibody-siRNA conjugates are prepared using Zn.

### Analysis of Anti-TfR1 Antibody-siRNA Conjugates Using Reduced Mass Spectrometry

### Results

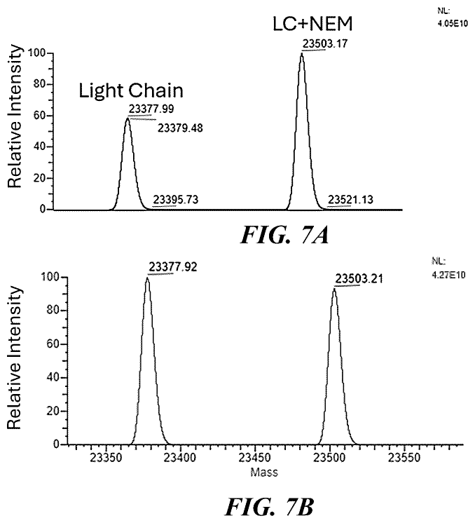

The antibody-siRNA conjugates prepared without Zn or with Zn were analyzed for N-ethylmaleimide (NEM) capping distribution of the DAR1 antibody-siRNA conjugate using reduced mass spectrometry. As shown in FIG. 7A, analysis of the mass chromatogram revealed that the majority of the light chains were capped with NEM when using the synthesis method without Zn. Interestingly, the analysis of the mass chromatogram for the intermediates prepared with Zn revealed that about half of the LC were capped with 1 NEM and half of the LC did not have any capped NEM (FIG. 7B). The data indicate that NEM was conjugated to the LC of the antibody and that siRNA was conjugated to the HC of the antibody. In addition, approximately 50% of the light chains were capped with NEM, which are consistent with the previous analytical results of the antibody-siRNA conjugate.

### Example 4: FabALACTICA Digests Analysis

### Materials

The antibody-siRNA conjugates are described in Example 1.

### Method

### FabALACTICA Digest Method

The location of siRNA conjugation was identified using FabALACTICA to digest the antibody heavy chain. The digested fragments were analyzed by size exclusion chromatography to separate Fab and Fc fractions with and without siRNA conjugated. Protein A purification was used to isolate Fc and Fab fractions (with and without siRNA) which were subsequently analyzed by SEC for peak identification. The bioconjugates were incubated at 37° C. overnight with FabALACTICA enzyme (Genovis), which is a cysteine protease that digests human IgG1 at a specific site above the hinge, thereby generating intact and homogenous Fab and Fc fragments. Protein A purification was performed using Nab Spin Columns, 0.2 ml (Thermo Scientific). The FabALACTICA digests were loaded on the columns in phosphate/sodium chloride binding buffer. The Fab and Fab-siRNA fragments were isolated by washing the column with the binding buffer. The Fc and Fc-siRNA fragments were eluted off the column in low pH glycine elution buffer and neutralized with high pH tris buffer. Collected fractions were concentrated with 0.5 ml 3 kDa spin filters.

### Size Exclusion Chromatographic (SEC) Method

Size Exclusion Chromatographic (SEC) method was used to determine the relative amounts of intact AOC, Fc-siRNA, Fab-siRNA, Fc, and Fab components. Separation was achieved by isocratic elution using a phosphate and potassium chloride running buffer. The protein-A fractions described above were analyzed to identify Fc and Fab components. The relative peak area of Fc-siRNA and Fab-siRNA components was used to determine the % bioconjugation site occupancy.
MPA: 50 mM sodium phosphate, 200 mM KCl pH 7.0
Detection: UV absorbance 280 nm
Column: BioResolve SEC mAb Column, 200 2.5 μm 7.8×300 mm (waters 186009441)
Flow rate=0.7 ml/min
Run Time=30 min

### Results

In order to determine the location of the conjugation of the siRNA on the antibody, the DART antibody-siRNA conjugates prepared with Zn or without Zn were analyzed using FabALACTICA, which is a cysteine protease that digests human IgG1 at one specific site above the hinge (KSCDKT (SEQ ID NO:331)/HTCPPC (SEQ ID NO:332)), resulting in intact Fab and Fc fragments.

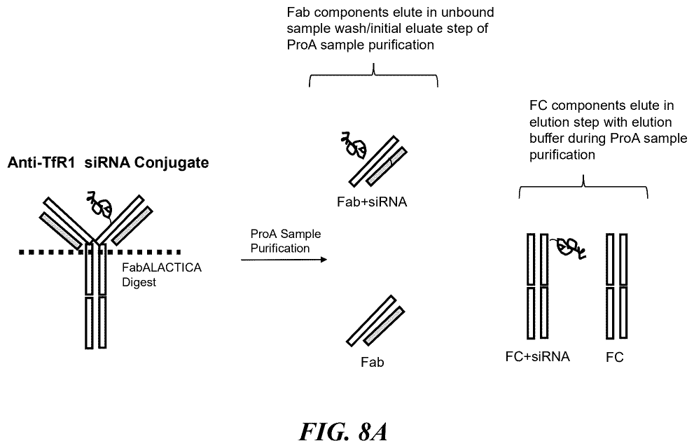

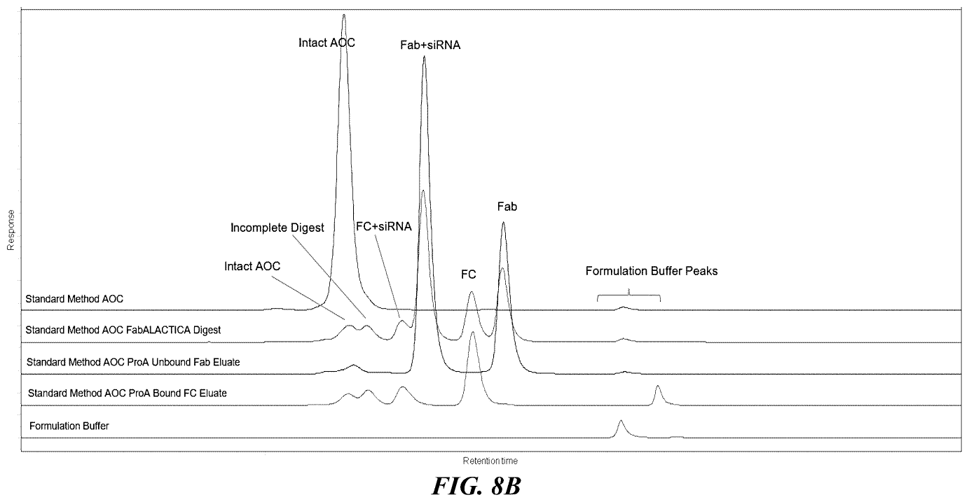

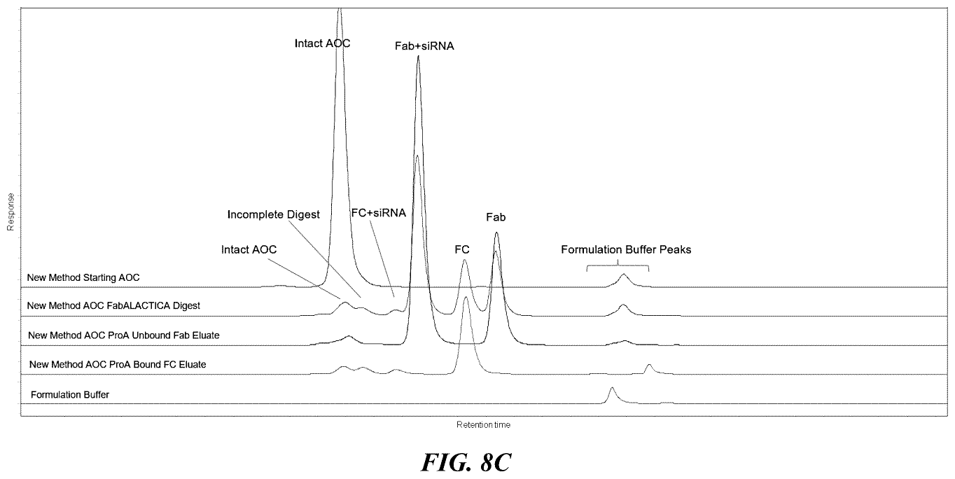

As illustrated in FIG. 8A, the conjugates were digested with FabALACTICA to obtain Fab and Fc fragments of the antibody-siRNA conjugates. The unbound fraction of the FabALACTICA digest passed through a protein A column contains the Fab arms, and the ProA bound peak from the protein A column contains the Fc portion. The intact antibody-siRNA conjugate, incomplete digested conjugates, Fc and Fab conjugate fragments were separated by size exclusion chromatography (SEC). As shown in FIGS. 8B-8C, the chromatograms were superimposed showing 5 different lines (top to bottom) representing (1) DAR1 antibody-siRNA conjugates (intact, not digested) (2) DAR1 antibody-siRNA conjugates digested with FabALACTICA enzyme (3) The digested DART antibody-siRNA conjugates containing Fab and Fab-siRNA portions that were unretained on the protein A column (4) The Fc and Fc-siRNA portions of the digested DAR1 antibody-siRNA conjugates that were eluted from the protein A column, (5) The formulation buffer by itself as a control to help identify the peak at the end of the SEC run.

Analysis of the different chromatograms of the conjugates digested with FabALACTICA enzyme revealed that the peaks observed with the antibody fragment contained the Fab arms of the digested conjugate indicating that the majority of the siRNA was conjugated to the Fab arms. In addition, the peaks in the antibody fragment containing the Fc region of the digested conjugate indicate that only a small amount of siRNA was conjugated to the FC portion of the antibody (13% for conjugate prepared without Zn and 4% of conjugates prepared with Zn). These results indicate that majority of siRNAs were conjugated onto the Fab portion of the conjugates for both methods. Although conjugates prepared without Zn has 13% of the siRNA conjugated to the Fc portion of the antibody, conjugates prepared with Zn only have 4% of siRNA conjugated to the Fc portion and 96% conjugated to the Fab portion of the antibody. These results confirm that the cysteine residue of the HC as the conjugation site for the linker attached to the siRNA.

Overall, the results confirm that siRNA are conjugated to the cysteine residue at the CH1 domain of the antibody for the DAR1 antibody-siRNA conjugated prepared with Zn.

### Example 5: Analysis of DAR2 Anti-TfR1 Antibody-siRNA Conjugates Reduced Capillary Gel Electrophoresis

### Materials and Methods

The DAR2 antibody-siRNA conjugates are described in Example 1 and the method of for reduced capillary gel electrophoresis is described in Example 2.

### Analysis of DAR2 Anti-TfR1 Antibody-siRNA Conjugates Using Reduced Capillary Gel Electrophoresis

### Results

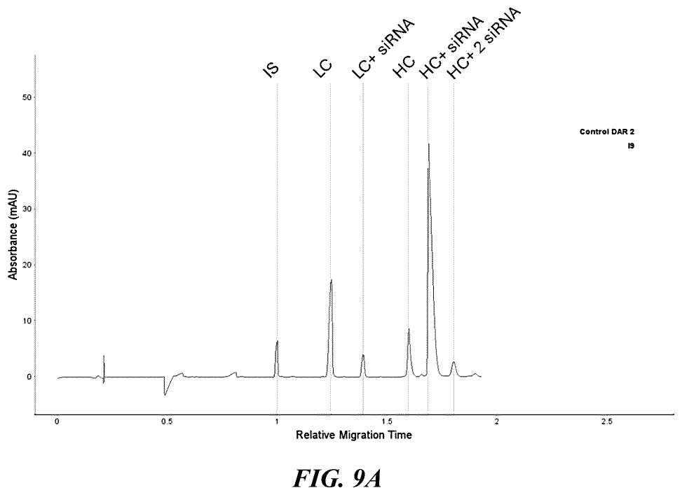

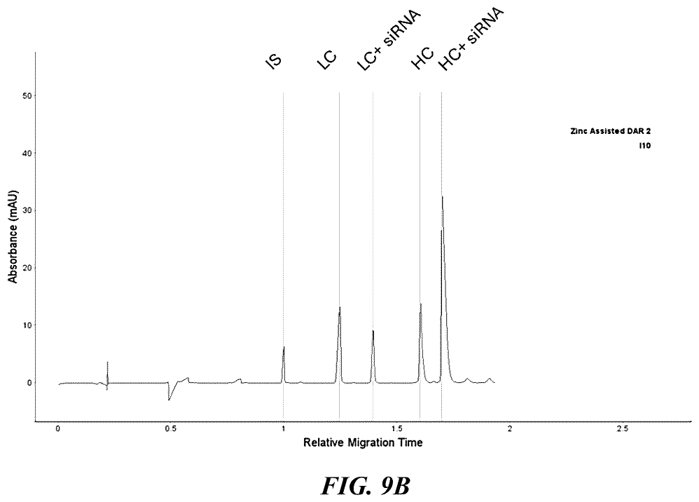

DAR2 antibody-siRNA conjugates were prepared with or without Zn. The isolated DAR2 conjugates were analyzed using reduced capillary gel electrophoresis (CE) in order to determine the levels of siRNA conjugated to the heavy chain (HC) and light chain (LC) of the antibody. Analysis of the electrophoresis chromatograms revealed that DAR2 antibody-siRNA conjugates prepared with and without Zn had siRNA conjugated to both LC and HC of the antibody. DAR2 antibody-siRNA conjugates prepared without Zn had more siRNA conjugated to the HC compared to DAR2 antibody-siRNA conjugates prepared with Zn (FIG. 9A). Higher siRNA conjugated to the HC suggests than there were numerous available cysteine residues on the HC of the conjugates prepared without Zn. However, DAR2 antibody-siRNA conjugates prepared with Zn had more siRNA conjugated to the LC compared to DAR2 conjugates prepared without Zn (FIG. 9B). These results are consistent with the formation of an intermediate in the presence of Zn that had only one available cysteine residue on the LC for conjugation with the linker of the siRNA.

Overall, these results indicate that DAR2 conjugates prepared with Zn have siRNA conjugated to LC of the antibody.

### Example 6: Analysis of Antibody-siRNA Conjugates with Strong Cation Exchange Chromatography (SCX)

### Material

The antibody-siRNA conjugates are described in Example 1.

### Method

### Strong Cation Exchange Chromatography Method

Strong cation-exchange chromatography (CEX-HPLC) with UV detection at 280 nm determines bioconjugate charge heterogeneity. In this example, CEX-HPLC was used to determine the purity of the bioconjugate by separating acidic and basic charge variants. It is also capable of monitoring heterogeneity induced by the bioconjugation process. Test samples were separated by pH gradient elution on a Sepax strong cation exchange column.
MPA: 10% CX-1 pH Gradient Buffer A (pH 5.6) (Thermo Cat #083273), 25 mM KCl
MPB: 10% CX-1 pH Gradient Buffer B (pH 10.2) (Thermo cat #0832275), 75 mM KCl
MPC: 10% CX-1 pH Gradient Buffer B (pH 10.2) (Thermo cat #0832275), 900 mM KCl
Detection: UV absorbance 280 nm
Column: Proteomix SCX (4.6×250 mm, 5 μm)
Flow rate=0.8 ml/min

### Gradient:

| Time | | | |
|---|---|---|---|
| (min) | % A | % B | % C |
| 0 | 100 | 0 | 0 |
| 3 | 100 | 0 | 0 |
| 42.9 | 53.5 | 46.5 | 0 |
| 43 | 0 | 0 | 100 |
| 53 | 0 | 0 | 100 |
| 53.1 | 100 | 0 | 0 |
| 65 | 100 | 0 | 0 |

Analysis of Antibody-siRNA Conjugates with Strong Cation Exchange Chromatography (SCX)

### Results

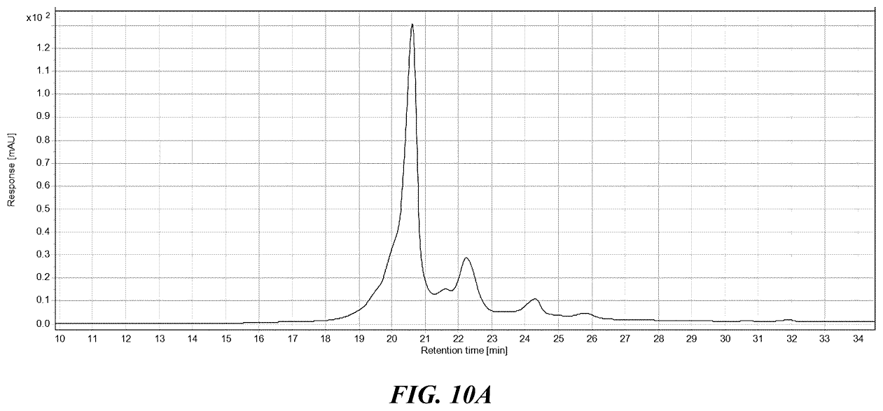

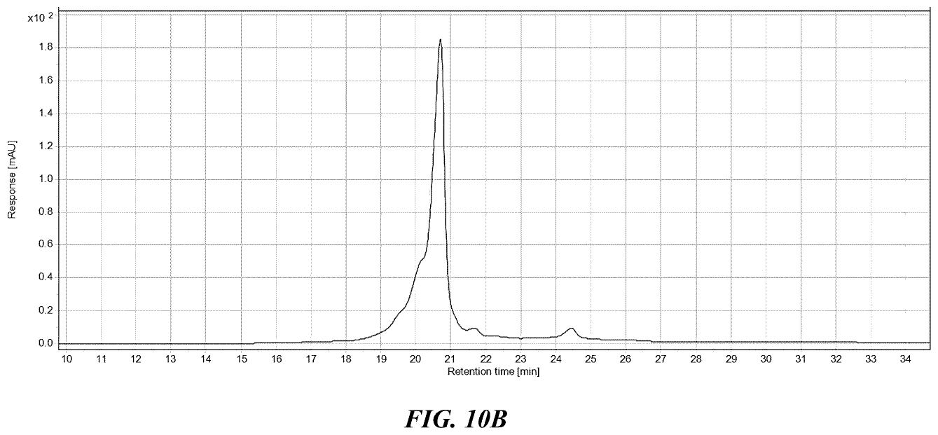

DAR1 anti-TfR1 antibody-siRNA conjugates prepared with or without Zn were analyzed for charge heterogeneity using strong cation exchange chromatography. As shown in FIGS. 10A-10B, analysis of the 2 chromatograms of the conjugates prepared with both methods revealed similar major peaks. However, the chromatogram for the conjugates prepared with Zn had fewer minor peaks indicating a more homogeneous final product. Overall, the anti-TfR1 antibody-siRNA conjugates prepared with Zn resulted in more homogenous final product mixture than anti-TfR1 antibody-siRNA conjugates prepared without Zn.

### Example 7: Preparation of Anti-TfR1 Antibody-siRNA Conjugate Having a Bismal Linker

### Materials

The antibody and siRNA are described in Example 1.

### Methods

The method for strong anion exchange (SAX) method for analyzing conjugates is described in Example 1.

Preparation of Antibody-siRNA Conjugates with Bismal Linkers

The anti-TfRT antibody-siRNA conjugate with the bismal linker (hTfR1.mAb-BisMal-siRNA) conjugate was prepared by adding 10 molar equivalents (meq) of TCEP (1.36 mM) to the solution containing the anti-TfRT monoclonal antibody at 20 g/L (0.136 mM) in 50 mM citrate and 300 mM sucrose at pH 6.5 and incubating at room temperature for 2 hours. The reaction mixture was diafiltered with a 50 kDa MWCO membrane and 8 diavolumes of 25 mM phosphate buffer at pH 7.5 bringing the antibody concentration to 10 mg/ml (0.0679 mM). 1.5 meq of ZnCl₂ (0.1018 mM) was added to the reaction mixture and incubated at room temperature for 30 minutes followed by 10 meq of dehydroascorbic acid (0.679 mM) incubated at room temperature for 2 hours. The reaction mixture was diafiltered with a 50 kDa MWCO membrane and 8 diavolumes of 25 mM phosphate buffer, pH 7.5 bringing the monoclonal antibody concentration to 10 g/L (0.0679 mM). EDTA (5 mM) was added to the reaction mixture followed by 1 meq of siRNA with a Bis-Mal-Lysine-PEG4 (BisMal) linker at the 5′ end of the passenger strand (0.0679 mm). The reaction mixture was incubated at room temperature for 30 minutes, followed by the addition of 10 meq of NEM (0.679 mM) and incubating at room temperature for 30 minutes. The reaction mixture was analyzed by strong anion exchange (SAX) chromatography to quantify the siRNA to antibody ratio (DAR) followed by purification by SAX chromatography to isolate antibody-siRNA conjugates with DART and tangential flow filtration with a 50 kDa MWCO membrane to concentrate the antibody-siRNA conjugates in citrate buffer.

Analysis of Antibody-siRNA Conjugates with Bismal Linkers Using Strong Anion Exchange (SAX) Chromatography

### Results

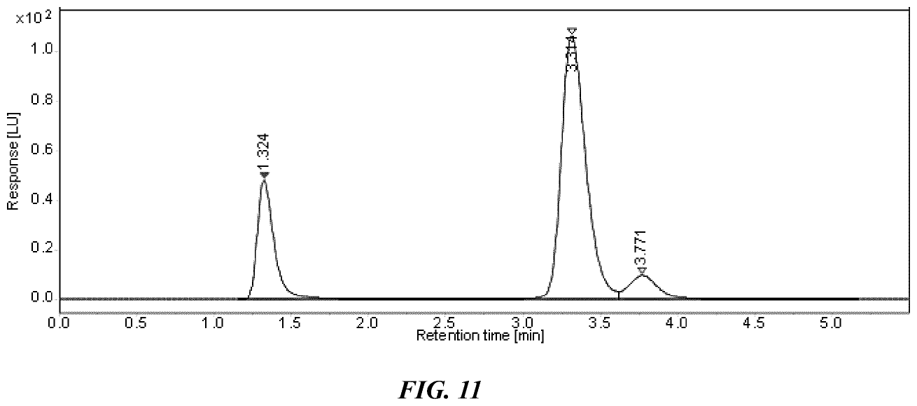

The reaction mixtures of antibody-siRNA conjugates prepared with Zn were analyzed by strong anion exchange chromatography (SAX) for DAR distribution of antibody-siRNA conjugates with bismal linkers (hTfR1.mAb-bismal-siRNA conjugates). As shown in FIG. 11, analysis of the chromatogram of the mixture obtained from the method of preparing the conjugate with Zn revealed a major peak representing the DAR1 antibody-siRNA conjugate. Relative quantitation of this peak revealed a DAR1 antibody-siRNA conjugate yield greater than 70% in the mixture in the conjugation method using a bismal linker that reacts with two different cysteine residues on the light and heavy chains of the antibody.

Overall, these results indicate that the conjugation of anti-TfR1 antibody to siRNA using Zn can be used for conjugation via various linkers including bridging linkers such as a bismal linker that reacts with two different cysteine residues.

### Example 8: Analysis Antibody NEM Conjugates by Mass Spectrometry

### Materials

The antibody is described in Example 1.

### Methods

The methods for mass spectrometry are described in Example 3.

### Preparation of Anti-TfR1 Antibody NEM Conjugates

Anti-human TfR1 antibody-NEM (hTfR1.mAb-NEM) conjugates were prepared by adding 10 molar equivalents (meq) of TCEP (1.36 mM) to the solution containing the anti-human TfR1 monoclonal antibody (hTfR1.mAb) at 20 g/L (0.136 mM) in 50 mM citrate and 300 mM sucrose at pH 6.5 and incubating at room temperature for 2 hours. The reaction mixture was diafiltered with a 50 kDa MWCO membrane and 8 diavolumes of 25 mM phosphate buffer, pH 7.5 bringing the antibody concentration to 10 mg/ml (0.0679 mm). 1.5 meq of ZnCl₂ (0.1018 mM) was added to the reaction mixture and incubated at room temperature for 30 minutes followed by 10 meq of dehydroascorbic acid (0.679 mM) incubated at room temperature for 2 hours. The reaction mixture was diafiltered with a 50 kDa MWCO membrane and 8 diavolumes of 25 mM phosphate buffer at pH 7.5 bringing the antibody concentration to 10 g/L (0.0679 mM). EDTA (5 mM) was added to the reaction mixture followed by 10 meq of NEM (0.679 mM). The reaction mixture was incubated at room temperature for 30 minutes, followed by tangential flow filtration (TFF) with a 50 kDa MWCO membrane to concentrate the antibody-NEM conjugate.

### Analysis of Antibody NEM Conjugates by Mass Spectrometry

### Results

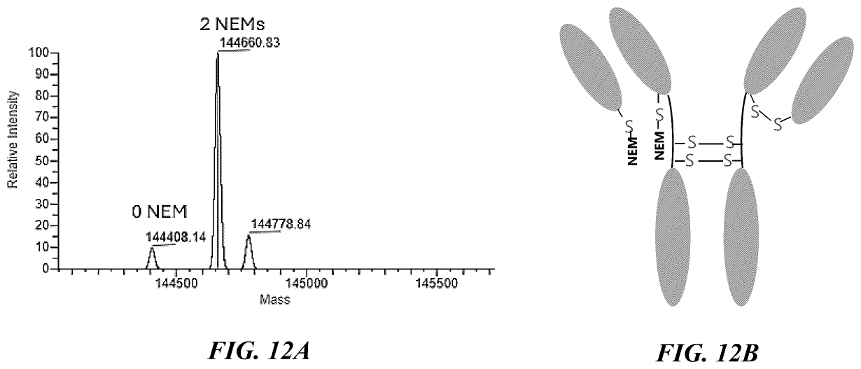

The antibody-NEM conjugates prepared with Zn were analyzed for N-ethylmaleimide (NEM) capping distribution of the DAR1 antibody-NEM conjugate using intact mass spectrometry. Instead of conjugating the antibody to siRNA via a linker, N-ethylmaleimide was used as a surrogate molecule. The small molecule NEM reacted with all available cysteine residues on the antibodies. As shown in FIG. 12A, the intact mass chromatogram revealed a main peak with the mass of 144660.83, which was the mass of the antibody attached to 2 NEM, representing approximately about 80% of the antibody-NEM conjugates. The presence of the antibody with 2 NEM as the most abundant species of antibody-NEM conjugates in the mixture suggested that the antibody had only one open disulfide formation available between the cysteine residues of the heavy and cysteine residues on the light chain of the antibody for reacting with NEM (FIG. 12B). The observation for the antibody-NEM conjugate was consistent with the one for the antibody-RNA conjugate prepared with the method using Zn.

Overall, the results of the NEM conjugate confirm that the conjugation of the antibody to siRNA or any other potential moieties with linkers, such as ASO, is via only an open disulfide bridge between the cysteine residues of the heavy and light chain of the antibody.

### Example 9: Preparation of Anti-TfR1 Antibody-siRNA Conjugate Purified with a Borate Buffer

### Materials and Methods

The antibody-siRNA conjugates and methods for SAX are described in Example 1.

### Preparation of Anti-TfR1 Antibody-siRNA Conjugate

The anti-TfR1 antibody-siRNA conjugate (hTfR1.mAb-MCC-siRNA) was prepared by adding 10 molar equivalents (meq) of TCEP (1.36 mM) to the solution containing antibody at 20 g/L (0.136 mM) in 50 mM citrate and 300 mM sucrose at pH 6.5 and incubating at room temperature for 2 hours. The reaction mixture was diafiltered with a 50 kDa MWCO membrane and 8 diavolumes of 25 mM phosphate buffer, pH 7.5 bringing the antibody concentration to 22.5 mg/ml (0.1527 mM). 1.5 meq of ZnCl₂ (0.229 mM) was added to the reaction mixture and incubated at room temperature for 20 minutes followed by 9 meq of dehydroascorbic acid (1.375 mM) incubated at room temperature for 110 min. A solution of 50 mM sodium tetraborate decahydrate (50 mM pH 7.5) was added to the antibody solution to reach a final concentration of 10 mM. After 10 minutes, a solution of EDTA (500 mM) was added to the antibody solution to reach a final EDTA concentration of 5 mM. After 10 minutes, 1.1 meq of siRNA with MCC linker attached to the 5′ end of the passenger strand was added to the antibody (0.168 mM). The reaction mixture was incubated at room temperature for 30 minutes, followed by the addition of 5 meq of NEM (0.764 mM) and incubation at room temperature for 30 minutes. The reaction mixture was analyzed by strong anion exchange (SAX) chromatography to quantify the siRNA to antibody ratio (DAR) followed by purification by SAX chromatography to isolate DAR1 hTfR1.mAb-MCC-siRNA and tangential flow filtration with a 50 kDa MWCO membrane to concentrate the hTfR1.mAb-MCC-siRNA in citrate buffer. As a control, the process above was repeated with 15 meq of L-ascorbic acid (2.29 mM) in place of sodium tetraborate.

TABLE 9

| | Zinc | DHAA | | Ascorbate | | | |
|---|---|---|---|---|---|---|---|
| | EQ | EQ | Borate | EQ | DAR0 | DAR1 | DAR2 |
| a | 1.5 | 10 | 10 mM | 0 | 8.3 | 80.1 | 11.6 |
| b | 1.5 | 9 | 10 mM | 0 | 8.9 | 80.7 | 10.4 |
| c | 1.5 | 8 | 10 mM | 0 | 8.8 | 80.6 | 10.6 |
| d | 1.5 | 7 | 10 mM | 0 | 9.0 | 79.9 | 11.1 |
| e | 1.5 | 6 | 10 mM | 0 | 10.5 | 77.7 | 11.8 |
| f | 1.5 | 5 | 10 mM | 0 | 11.9 | 74.9 | 13.2 |
| g | 1.5 | 7 | 0 | 15 | 29.4 | 43.8 | 26.8 |
| h | 1.25 | 7 | 10 mM | 0 | 9.5 | 80.0 | 10.5 |

Analysis of the Preparation of Anti-TfR1 Antibody-siRNA Conjugate with Zn and Borate

### Results

The reaction mixture of antibody-siRNA conjugates prepared with Zn were analyzed by strong anion exchange chromatography (SAX) for DAR distribution of antibody-siRNA conjugates.

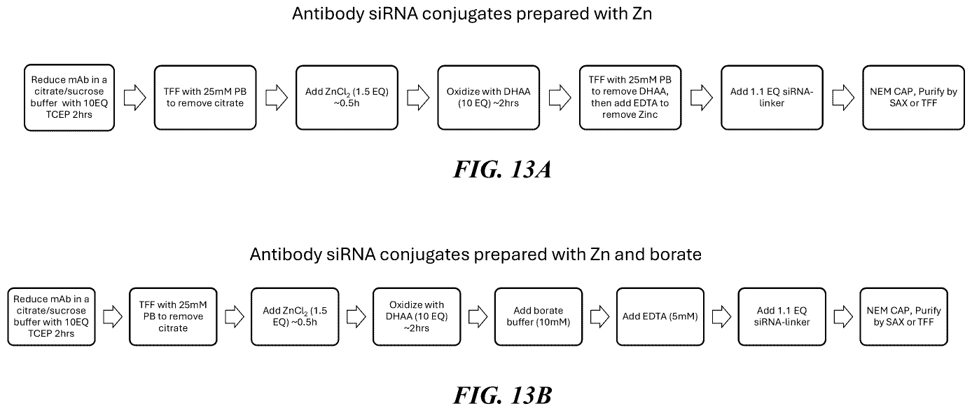

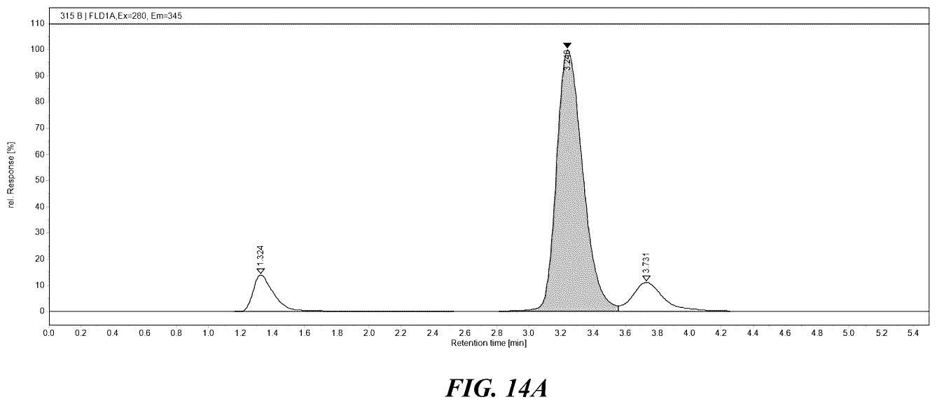

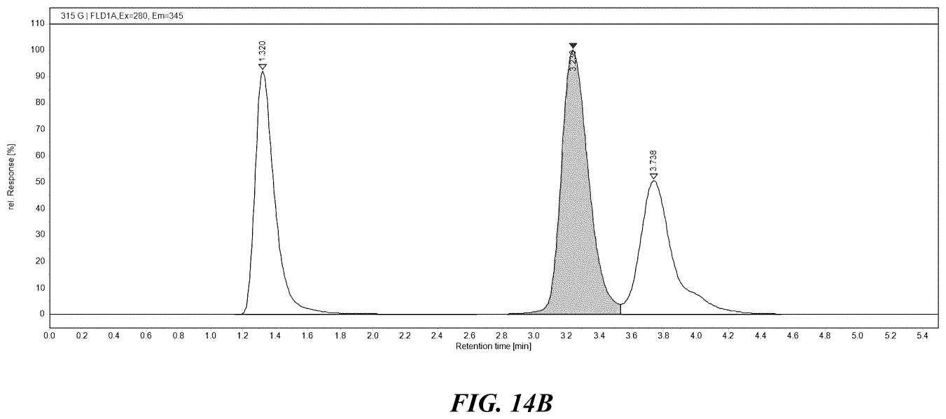

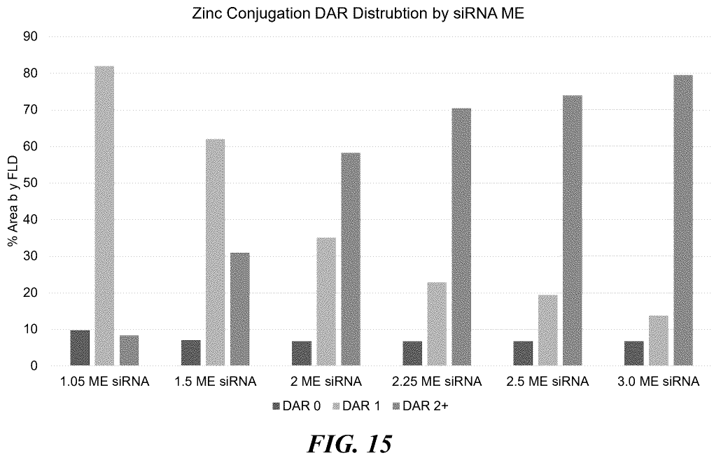

In the method of preparing antibody-siRNA conjugates with Zn, DHAA was added to the mixture and had to be removed from the mixture (FIG. 13A). Tangential flow filtration (TFF) was used to remove the excess DHAA. The purification step with TFF may take up to 24 hours, depending on the scale of the manufacturing of the conjugates. Without these 2 Cys residues, the siRNA-MCC was not able to react with the antibody resulting in decreases in DAR1 yields.

In order to decrease the purification time while maintaining DART yields, a borate buffer (10 mM final concentration) was substituted for TFF (FIG. 13B). The addition of borate to the antibody solution decreased the formation of disulfide bonds between cysteine residues due to the oxidizing agent DHAA. With the removal of DHAA, Zn was able to be sequestered with EDTA from the antibody, which can be conjugated to a siRNA-MCC with open cysteine residues.

As shown in Table 9, DART hTfR1.mAb-MCC-siRNA yield was significantly reduced with the lowest DART yield around 44% in the sample with DHAA and without any borate while DART yield of 80% was the highest in the presence of borate. The chromatograms of the antibody-siRNA conjugate mixture prepared with condition b has a large peak representing DART antibody-siRNA conjugates (FIG. 14A). However, analysis chromatogram of the antibody-siRNA conjugate mixture prepared with condition g revealed 3 peaks of similar sizes representing DAR0, DAR1, and DAR2 antibody-siRNA conjugates (FIG. 14B). Comparison of the chromatograms under these 2 different confirms the DART yield disclosed in Table 9.

The use of borate showed the unexpected results of significantly reducing time needed to manufacture antibody-siRNA conjugates while maintaining a DART antibody-siRNA conjugate yield higher than 80%.

### Example 10: Preparation of Anti-Her2 Antibody-siRNA Conjugate Having a MCC Linker

### Materials

The siRNA is described in Example 1. The human IgG1 antibody used is Trastuzamab (anti-HER2)

### Methods

The method for strong anion exchange (SAX) method for analyzing conjugates is described in Example 1.

### Preparation of Trastuzamab-siRNA Conjugate Using Zinc

The anti-HER2 antibody-siRNA conjugate with the MCC linker (HER2.mAb-MCC-siRNA) conjugate was prepared by adding 10 molar equivalents (meq) of TCEP (1.36 mM) to the solution containing the anti-HER2 monoclonal antibody at 20 g/L (0.136 mM) in histidine buffer pH 6 and incubating at room temperature for 2 hours. The reaction mixture was diafiltered with a 50 kDa MWCO membrane and 8 diavolumes of 25 mM phosphate buffer at pH 7.5 bringing the antibody concentration to 10 mg/ml (0.0679 mM). 1.5 meq of ZnCl₂ (0.1018 mM) was added to the reaction mixture and incubated at room temperature for 30 minutes followed by 10 meq of dehydroascorbic acid (0.679 mM) incubated at room temperature for 2 hours. The reaction mixture was diafiltered with a 50 kDa MWCO membrane and 8 diavolumes of 25 mM phosphate buffer, pH 7.5 bringing the monoclonal antibody concentration to 10 g/L (0.0679 mM). EDTA (5 mM) was added to the reaction mixture followed by 1 meq of siRNA with a MCC linker at the 5′ end of the passenger strand (0.0679 mM). The reaction mixture was incubated at room temperature for 30 minutes, followed by the addition of 2 meq of NEM (0.136 mM) and incubating at room temperature for 30 minutes. The reaction mixture was analyzed by strong anion exchange (SAX) chromatography to quantify the siRNA to antibody ratio (DAR) followed by purification by SAX chromatography to isolate antibody-siRNA conjugates with DAR1 and tangential flow filtration with a 50 kDa MWCO membrane to concentrate the antibody-siRNA conjugates in phosphate buffer

Preparation of Trastuzamab-siRNA Conjugate without Zinc

The anti-HER2 antibody-siRNA conjugate with the MCC linker (HER2.mAb-MCC-siRNA) conjugate was prepared by adding EDTA (2 mM) and 2 molar equivalents (meq) of TCEP to the solution containing the anti-HER2 monoclonal antibody at 20 g/L (0.136 mM) in histidine buffer pH 6 and incubating at room temperature for 2 hours. To the solution, 1 meq of siRNA with a MCC linker at the 5′ end of the passenger strand was added. The reaction mixture was incubated at room temperature for 30 minutes, followed by the addition of 10 meq of NEM and incubating at room temperature for 30 minutes. The reaction mixture was analyzed by strong anion exchange (SAX) chromatography to quantify the siRNA to antibody ratio (DAR) followed by purification by SAX chromatography to isolate antibody-siRNA conjugates with DART and tangential flow filtration with a 50 kDa MWCO membrane to concentrate the antibody-siRNA conjugates in phosphate buffer.

Analysis of Antibody-siRNA Conjugates with MCC Linkers Using Strong Anion Exchange (SAX) Chromatography

### Results

The reaction mixtures of Trastuzumab-siRNA conjugates prepared with Zn were analyzed by strong anion exchange chromatography (SAX) for DAR distribution of antibody-siRNA conjugates with MCC linkers (HER2.mAb-MCC-siRNA conjugates). Relative quantitation of each DAR species revealed a DAR1 antibody-siRNA conjugate yield greater than 70% in the mixture.

Overall, these results indicate that the process for generating DAR1 antibody-oligonucleotide conjugates can be applied to many antibodies.

TABLE 10

| | Herceptin- | Herceptin-siRNA |
|---|---|---|
| | siRNA Zn | Conjugation |
| | Conjugation | (no Zinc) |
| DAR 0 | 13 | 14.1 |
| (% AUC) | | |
| DAR 1 | 81 | 62.8 |
| (% AUC) | | |
| DAR 2 | 6 | 21.1 |
| (% AUC) | | |
| DAR 3 | 0 | 2 |
| (% AUC) | | |

### Example 11: Preparation of DAR2 Anti-TfR1 Antibody-siRNA Conjugate Having a MCC Linker

### Materials

The antibody and siRNA are described in Example 1.

### Methods

The method for strong anion exchange (SAX) method for analyzing conjugates is described in Example 1.

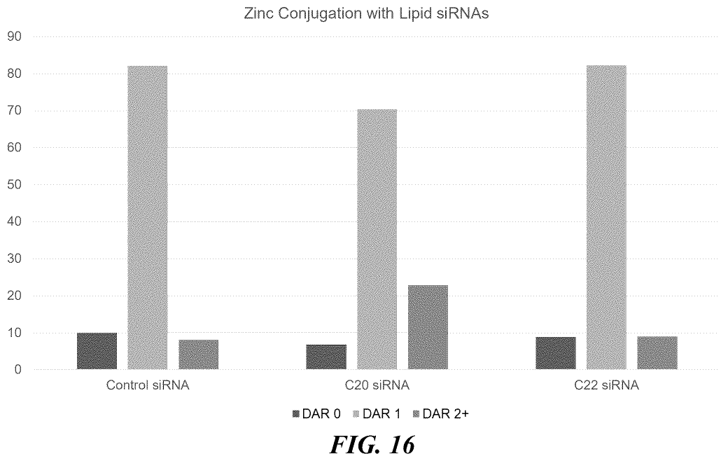

DAR2 antibody-siRNA conjugates were prepared by adding 10 meq of TCEP (1.36 mM) to the solution containing human IgG1 monoclonal antibody targeting hTfR1 (hTfR1.mAb) at 20 g/L (0.136 mM) in a buffer solution comprising 50 mM citrate and 300 mM sucrose at pH 6.5 and incubated at room temperature for 2 hours. The reaction mixture was diafiltered with a 50 kDa MWCO membrane and 8 diavolumes of 25 mM phosphate buffer at pH 7.5, bringing the hTfR1.mAb concentration to 10 mg/ml (0.0679 mM). 1.5 meq of ZnCl₂ (0.1018 mM) was added to the reaction mixture and incubated at room temperature for 30 minutes followed by 10 meq of DHAA (0.679 mM) incubated at room temperature for 1 hour. The reaction mixture was diafiltered with a 50 kDa MWCO membrane and 8 diavolumes of 25 mM phosphate buffer, pH 7.5, bringing the hTfR1.mAb concentration to 10 g/L (0.0679 mM). Then EDTA was added to a final concentration of 5 mM, after incubation at room temperature for 15 minutes, 1.05, 1.5, 2.0, 2.25, 2.5, or 3 meq of siRNA with a MCC linker at the 5′ end of the passenger strand was added. The reaction mixture was incubated at room temperature for 30 minutes, followed by the addition of 2 meq of NEM and incubation at room temperature for 30 minutes. The reaction mixture was analyzed by SAX chromatography to quantify the siRNA to antibody ratio (DAR). Reaction mixtures with greater than 70% DAR2 were achieved with this method as shown in FIG. 16 and Table 11.

The results indicate that this method can be used to selectively make DAR2 AOCs by increasing the meq of siRNA used.

TABLE 11

| | 1.05 ME | 1.5 ME | 2 ME | 2.25 ME | 2.5 ME | 3 ME |
|---|---|---|---|---|---|---|
| | siRNA | siRNA | siRNA | siRNA | siRNA | siRNA |
| DAR 0 | 10 | 7 | 7 | 7 | 7 | 7 |
| (% AUC) | | | | | | |
| DAR 1 | 82 | 62 | 35 | 23 | 19 | 14 |
| (% AUC) | | | | | | |
| DAR 2 | 8 | 31 | 58 | 70 | 74 | 79 |
| (% AUC) | | | | | | |

### Example 12: Preparation of DAR1 Anti-TfR1 Antibody-siRNA-Lipid Conjugate Having a MCC Linker

### Materials

The antibody is described in Example 1.

The siRNAs used are: control siRNA and two additional siRNAs with C20 or C22 alkyl chains off of the 2′ position of the 3rd base on the passenger strand.

TABLE 12

| | | SEQ | | SEQ |
|---|---|---|---|---|
| | | ID | | ID |
| siRNA | Guide Strand | NO: | Passenger Strand | NO: |
| Control | m(U)[ps].[fl2r](U)[ps].m(C)p.m | 1 | [5am6_p_m](C)[ps].m(C)[ps].m(C)p. | 2 |
| siRNA | (G)p.m(A)p.[fl2r](A)p.m(G)p.m(A) | | m(U)p.m(A)p.m(G)p.[fl2r](A)p.[fl2r] | |
| | p.m(C)p.m(A)p.m(G)p.m(U)p.m | | (A)p.[fl2r](C)p.m(U)p.m(G)p.m(U)p. | |
| | (U)p.[fl2r](C)p.m(U)p.[fl2r](A)p. | | m(C)p.m(U)p.m(U)p.m(C)p.m(G)[ps]. | |
| | m(G)p.m(G)p.m(G)[ps].m(U)[ps]. | | m(A)[ps].m(A) | |
| | m(U) | | | |
| siRNA with | [5vp_una2moe](U)[ps].[fl2r](U) | 3 | [5am6_p_m](C)[ps].m(C)[ps].[rc20] | 4 |
| C20 alky1 | [ps].m(C)p.m(G)p.m(A)p.[fl2r](A) | | (C)p.m(U)p.m(A)p.m(G)p.[fl2r](A)p. | |
| chain | p.m(G)p.m(A)p.m(C)p.m(A)p.m | | [fl2r](A)p.[fl2r](C)p.m(U)p.m(G)p.m | |
| | (G)p.m(U)p.m(U)p.[fl2r](C)p.m(U) | | (U)p.m(C)p.m(U)p.m(U)p.m(C)p.m | |
| | p.[fl2r](A)p.m(G)p.m(G)p.m(G) | | (G)[ps].m(A)[ps].m(A) | |
| | [ps].m(U)[ps].m(U) | | | |
| siRNA with | [5vp_una2moe](U)[ps].[fl2r](U) | 3 | [5am6_p_m](C)[ps].m(C)[ps].[rc22] | 5 |
| C22 alkyl | [ps].m(C)p.m(G)p.m(A)p.[fl2r](A) | | (C)p.m(U)p.m(A)p.m(G)p.[fl2r](A)p. | |
| chain | p.m(G)p.m(A)p.m(C)p.m(A)p.m | | [fl2r](A)p.[fl2r](C)p.m(U)p.m(G)p.m | |
| | (G)p.m(U)p.m(U)p.[fl2r](C)p.m(U) | | (U)p.m(C)p.m(U)p.m(U)p.m(C)p.m | |
| | p.[fl2r](A)p.m(G)p.m(G)p.m(G) | | (G)[ps].m(A)[ps].m(A) | |
| | [ps].m(U)[ps].m(U) | | | |

5vp_una2moe 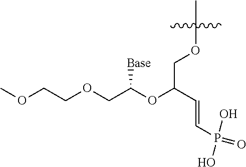
5am6_p_m 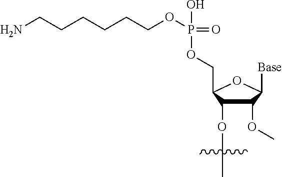
rc20 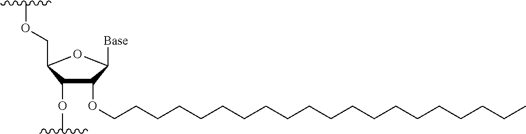
rc22

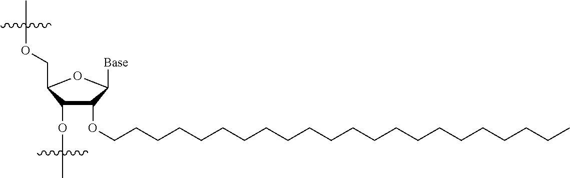

In this table, m indicates the 2′-O-methyl (2′-OMe) ribose modification; [fl2r] indicates the 2′-deoxy-2′-fluoro(2′-F) ribose modification; [ips] indicates phosphorothioate (PS) linkage; p indicates phosphodiester (PO) linkage

### Methods

The method for strong anion exchange (SAX) method for analyzing conjugates is described in Example 1.

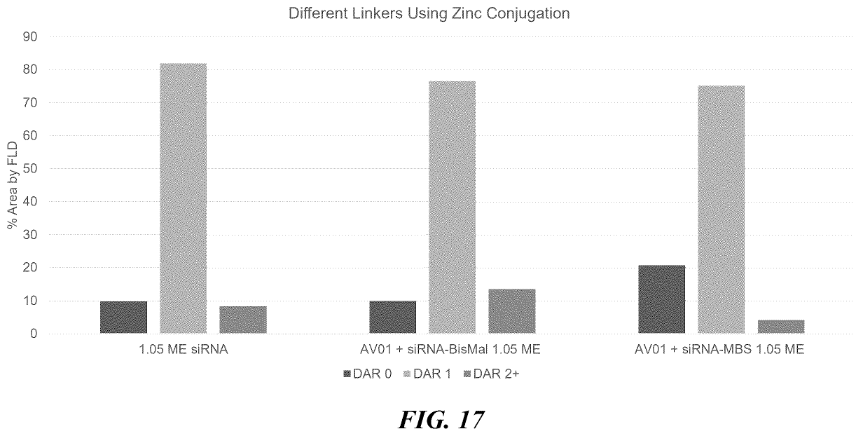

In this example, antibody-siRNA conjugates were prepared using a cysteine conjugation method using Zn. Antibody-siRNA conjugates were prepared by adding 10 molar equivalents (meq) of TCEP (1.36 mM) to the solution containing hTfR1.mAb at 20 g/L (0.136 mM) in a buffer solution comprising 50 mM citrate and 300 mM sucrose at pH 6.5 and incubated at room temperature for 2 hours. The reaction mixture was diafiltered with a 50 kDa MWCO membrane and 8 diavolumes of 25 mM phosphate buffer at pH 7.5, bringing the hTfR1.mAb concentration to 10 mg/ml (0.0679 mM). 1.5 meq of ZnCl₂ (0.1018 mM) was added to the reaction mixture and incubated at room temperature for 30 minutes followed by 10 meq of DHAA (0.679 mM) incubated at room temperature for 1 hour. The reaction mixture was diafiltered with a 50 kDa MWCO membrane and 8 diavolumes of 25 mM phosphate buffer, pH 7.5, bringing the hTfR1.mAb concentration to 10 g/L (0.0679 mM). Then EDTA was added to a final concentration of 5 mM, after incubation at room temperature for 15 minutes, 1.05 meq of siRNA with a MCC linker at the 5′ end of the passenger strand and an alkyl chain (C20, or C22) off of the 2′ position of the 3^{rd} nucleotide from the 5′ end of the passenger strand was added. The reaction mixture was incubated at room temperature for 30 minutes, followed by the addition of 2 meq of NEM and incubation at room temperature for 30 minutes. The reaction mixture was analyzed by SAX chromatography to quantify the siRNA to antibody ratio (DAR). Reaction mixtures with greater than 70% DAR1 were achieved with this method as shown in FIG. 17 and Table 13. The results indicate that this method can be used to make DAR1 lipid-siRNA AOCs

TABLE 13

| | AV01- | AV01-siRNA- | AV01-siRNA- |
|---|---|---|---|
| | siRNA Zn | C20 Zn | C22 Zn |
| | Conjugation | Conjugation | Conjugation |
| DAR 0 | 10 | 7 | 9 |
| (% AUC) | | | |
| DAR 1 | 82 | 70 | 82 |
| (% AUC) | | | |
| DAR 2 | 8 | 23 | 9 |
| (% AUC) | | | |

### Example 13: Preparation of Anti-TfR1 Antibody-siRNA Conjugate with Alternative Linkers

### Materials

The antibody and siRNA are described in Example 1.

### Methods

The method for strong anion exchange (SAX) method for analyzing conjugates is described in Example 1.

Preparation of Antibody-siRNA Conjugates with MCC, Bismal and MBS Linkers

The anti-TfR1 antibody-siRNA conjugate with the MCC, Bismal, and MBS linkers (hTfR1.mAb-MCC-siRNA, hTfR1.mAb-Bismal-siRNA, hTfR1.mAb-MBS-siRNA) conjugate was prepared by adding 10 meq of TCEP (1.36 mM) to the solution containing the anti-TfR1 monoclonal antibody at 20 g/L (0.136 mM) in 50 mM citrate and 300 mM sucrose at pH 6.5 and incubating at room temperature for 2 hours. The reaction mixture was diafiltered with a 50 kDa MWCO membrane and 8 diavolumes of 25 mM phosphate buffer at pH 7.5 bringing the antibody concentration to 10 mg/ml (0.0679 mM). 1.5 meq of ZnCl₂ (0.1018 mM) was added to the reaction mixture and incubated at room temperature for 30 minutes followed by 10 meq of dehydroascorbic acid (0.679 mM) incubated at room temperature for 1 hour. The reaction mixture was diafiltered with a 50 kDa MWCO membrane and 8 diavolumes of 25 mM phosphate buffer, pH 7.5 bringing the monoclonal antibody concentration to 10 g/L (0.0679 mM). EDTA (final concentration of 5 mM) was added to the reaction mixture followed by 1 meq of siRNA with a MCC, Bismal, or MBS linker at the 5′ end of the passenger strand (0.0679 mM). The reaction mixture was incubated at room temperature for 30 minutes, followed by the addition of 2 meq of NEM and incubating at room temperature for 30 minutes. The reaction mixture was analyzed by SAX chromatography to quantify the DAR distribution.

Analysis of Antibody-siRNA Conjugates with Bismal Linkers Using Strong Anion Exchange (SAX) Chromatography

### Results

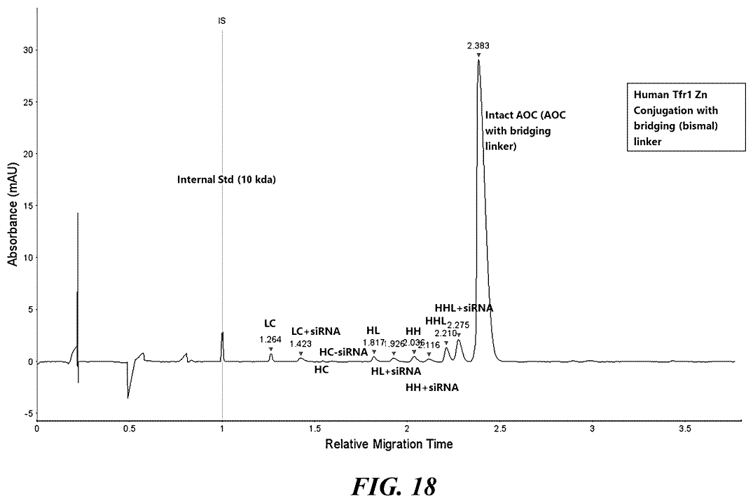

The reaction mixtures of antibody-siRNA conjugates prepared with Zn were analyzed by strong anion exchange chromatography (SAX) for DAR distribution of antibody-siRNA conjugates with MCC, Bismal and MBS linkers. As shown in FIG. 17 and Table 14, all three linkers resulted in greater than 70% DAR1 in the reaction mixture. Additionally, CGE analysis of the antibody-oligonucleotide conjugate (AOC) made with the Bismal linker and the zinc conjugation method showed that the primary peak was the intact AOC with one siRNA attached, indicating that the bismal linker was bridging the Cys between the heavy chain and light chain (FIG. 18), compared to an AOC made with MCC that had a primary peak of heavy-heavy-light-siRNA and a separate peak for LC (FIG. 4C) that was not covalently attached.

Overall, these results indicate that the conjugation of anti-TfR1 antibody to siRNA using Zn can be used for conjugation via various linkers including bridging linkers, such as a bismal linker that reacts with two different cysteine residues.

TABLE 14

| | AV01-MCC- | AV01-BisMal- | AV01-MBS- |
|---|---|---|---|
| | siRNA Zn | siRNA Zn | siRNA Zn |
| | Conjugation | Conjugation | Conjugation |
| DAR 0 | 10 | 10 | 21 |
| (% AUC) | | | |
| DAR 1 | 82 | 77 | 75 |
| (% AUC) | | | |
| DAR 2 | 8 | 13 | 4 |
| (% AUC) | | | |

### Example 14: Preparation of Anti-TfR1 Antibody-siRNA Conjugates without TFF Removal of DHAA

### Materials and Methods

The antibody-siRNA conjugates and methods for SAX are described in Example 1.

### Preparation of Anti-TfR1 Antibody-siRNA Conjugate

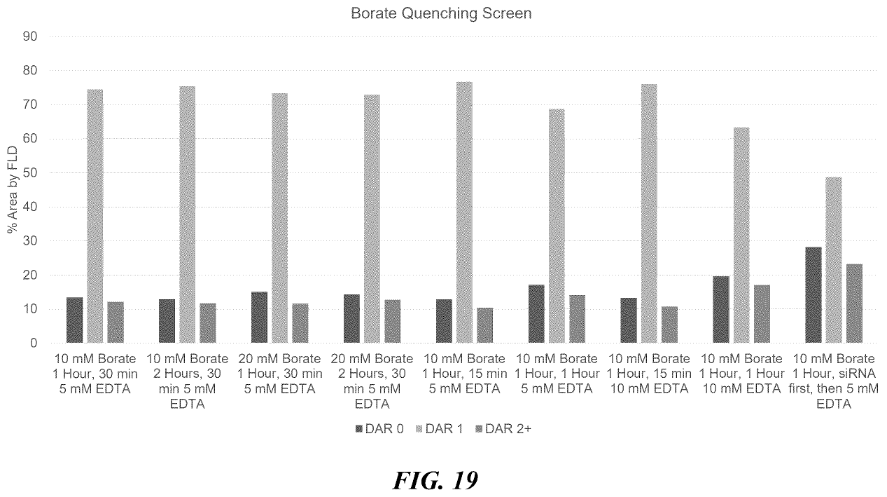

The anti-TfR1 antibody-siRNA conjugate (hTfR1.mAb-MCC-siRNA) was prepared by adding 10 molar equivalents (meq) of TCEP (1.36 mM) to the solution containing antibody at 20 g/L (0.136 mM) in 50 mM citrate and 300 mM sucrose at pH 6.5 and incubating at room temperature for 2 hours. The reaction mixture was diafiltered with a 50 kDa MWCO membrane and 8 diavolumes of 25 mM phosphate buffer, pH 7.5 bringing the antibody concentration to 22.5 mg/ml (0.1527 mM). 1.5 meq of ZnCl₂ (0.229 mM) was added to the reaction mixture and incubated at room temperature for 20 minutes followed by 10 meq of dehydroascorbic acid (1.375 mM) incubated at room temperature for 60 min. A solution of 250 mM sodium tetraborate decahydrate (250 mM pH 7.5) was added to the antibody solution to reach a final concentration of 10 or 20 mM. After 60 or 120 minutes, a solution of EDTA (500 mM) was added to the antibody solution to reach a final EDTA concentration of 5 or 10 mM. After 15, 30, or 60 minutes, 1.1 meq of siRNA with MCC linker attached to the 5′ end of the passenger strand was added to the antibody. In the last reaction, the siRNA was added prior to the addition of EDTA. The reaction mixture was incubated at room temperature for 30 minutes, followed by the addition of 2 meq of NEM and incubation at room temperature for 30 minutes. The reaction mixture was analyzed by strong anion exchange (SAX) chromatography to quantify the siRNA to antibody ratio (DAR) (FIG. 19, Table 15).

These results indicate that the second TFF step can be replaced by inactivation of the oxidizing agent but timing of each step will impact yields.

TABLE 15

| Borate and EDTA | DAR 0 | DAR 1 | DAR 2 |
|---|---|---|---|
| Conditions | (% AUC) | (% AUC) | (% AUC) |
| 10 mM Borate 1 Hour, | 13 | 75 | 12 |
| 30 min 5 mM EDTA | | | |
| 10 mM Borate 2 Hours, | 13 | 75 | 12 |
| 30 min 5 mM EDTA | | | |
| 20 mM Borate 1 Hour, | 15 | 73 | 12 |
| 30 min 5 mM EDTA | | | |
| 20 mM Borate 2 Hours, | 14 | 73 | 13 |
| 30 min 5 mM EDTA | | | |
| 10 mM Borate 1 Hour, | 13 | 77 | 10 |
| 15 min 5 mM EDTA | | | |
| 10 mM Borate 1 Hour, | 17 | 69 | 14 |
| 1 Hour 5 mM EDTA | | | |
| 10 mM Borate 1 Hour, | 13 | 76 | 11 |
| 15 min 10 mM EDTA | | | |
| 10 mM Borate 1 Hour, | 20 | 63 | 17 |
| 1 Hour 10 mM EDTA | | | |
| 10 mM Borate 1 Hour, | 28 | 49 | 23 |
| siRNA first, then | | | |
| 5 mM EDTA | | | |

### Example 15: Preparation of Anti-TfR1 Antibody-siRNA Conjugates with Alternate Reducing Agents

### Materials and Methods

The antibody-siRNA conjugates and methods for SAX are described in Example 1.

### Preparation of Anti-TfR1 Antibody-siRNA Conjugate

The anti-TfR1 antibody-siRNA conjugate (hTfR1.mAb-MCC-siRNA) was prepared by adding 10 molar equivalents (meq) of tris(2-carboxyethyl)phosphine (TCEP) or dithiothreitol (DTT) to the solution containing antibody at 24 g/L in 50 mM citrate and 300 mM sucrose at pH 6.5 and incubating at room temperature for 18 hours. The reaction mixture was diafiltered with a 50 kDa MWCO membrane and 8 diavolumes of 25 mM phosphate buffer, pH 7.5 bringing the antibody concentration to 22.5 mg/ml. 1.5 meq of ZnCl₂ was added to the reaction mixture and incubated at room temperature for 30 minutes followed by 10 meq of L-dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 22.5 mg/ml A solution of EDTA (500 mM) was added to the antibody solution to reach a final EDTA concentration of 5 mM. After 15 minutes, 1.1 meq of siRNA with MCC linker attached to the 5′ end of the passenger strand was added to the antibody. The reaction mixture was incubated at room temperature for 30 minutes, followed by analysis by strong anion exchange (SAX) chromatography to quantify the siRNA to antibody ratio (DAR). The results indicate that many reducing agents can be used in this process.

### SAX Analysis Results:

| Reductant | Equivalents | mAb | D1 | D2+ |
|---|---|---|---|---|
| TCEP | 10 | 16.5 | 78.8 | 4.7 |
| DTT | 10 | 15.6 | 79.2 | 5.2 |

### Example 16: Preparation of Palivizumab-siRNA Conjugates with Zinc Method

### Materials

The siRNA used was described in Example 1. The antibody used was Palivizumab.

### Preparation of Palivizumab-siRNA Conjugate

The Palivizumab-siRNA conjugate (anti-human IgG1 isotype control.mAb-MCC-siRNA) was prepared by adding 10 meq of TCEP to the solution containing antibody at 11.83 g/L in 20 mM Histidine Acetate, 0.15 M NaCl, pH 5.5 and incubating at room temperature for 18 hours. The reaction mixture was diafiltered with a 50 kDa MWCO membrane and 8 diavolumes of 25 mM phosphate buffer, pH 7.5 bringing the antibody concentration to 12.10 mg/ml. 1.5 meq of ZnCl₂ was added to the reaction mixture and incubated at room temperature for 30 minutes followed by 10 meq of dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 14.08 g/L. A solution of EDTA (500 mM) was added to the antibody solution to reach a final EDTA concentration of 5 mM. After 15 minutes, 1.05 meq of siRNA with MCC linker attached to the 5′ end of the passenger strand was added to the antibody. The reaction mixture was incubated at room temperature for 30 minutes, followed by analysis by strong anion exchange (SAX) chromatography to quantify the siRNA to antibody ratio (DAR). Following this 2 meq of NEM (in DMSO at 10 g/L) were added to the reaction mixture and incubated at room temperature for 30 minutes, and purified by SAX chromatography. The isolated DAR1 was analyzed by SAX, SEC and CE.

The results show that the zinc conjugation method results in improved yields and a comparable conjugation distribution for multiple antibodies, with the primary species on CE being the HHL-siRNA.

### SAX AUC Results

| mAb | D1 | D2 |
|---|---|---|
| 9.3 | 72.4 | 18.3 |

### Example 17: Preparation of DAR 2 Antibody-siRNA Conjugate with Two Different siRNA Payloads

### Materials and Methods

The human Tfr1 antibody used is described in Example 1.

The sequences of the siRNAs used in Example 17 are shown in Table 16.

TABLE 16

| | | SEQ | | SEQ |
|---|---|---|---|---|
| | | ID | | ID |
| siRNA | Guide Strand | NO: | Passenger Strand | NO: |
| RNA1 | m(U)[ps].[fl2r](U)[ps].m(C)p.m | 1 | [5am6_p_m](C)[ps].m(C)[ps].m(C)p. | 2 |
| | (G)p.m(A)p.[fl2r](A)p.m(G)p.m(A) | | m(U)p.m(A)p.m(G)p.[fl2r](A)p.[fl2r] | |
| | p.m(C)p.m(A)p.m(G)p.m(U)p.m | | (A)p.[fl2r](C)p.m(U)p.m(G)p.m(U)p. | |
| | (U)p.[fl2r](C)p.m(U)p.[fl2r](A)p. | | m(C)p.m(U)p.m(U)p.m(C)p.m(G)[ps]. | |
| | m(G)p.m(G)p.m(G)[ps].m(U)[ps]. | | m(A)[ps].m(A) | |
| | m(U) | | | |
| RNA2 | [5vp_una2moe](U)[ps].[fl2r](U) | 11 | [5am6_p_m](C)[ps].m(U)[ps].m(A)p. | 12 |
| | [ps].m(A)p.m(A)p.m(A)p.[fl2r](A) | | m(U)p.m(G)p.m(A)p.[fl2r](C)p.[fl2r] | |
| | p.m(U)p.m(C)p.m(U)p.m(A)p.m | | (U)p.[fl2r](G)p.m(U)p.m(A)p.m(G) | |
| | (C)p.m(A)p.m(G)p.[fl2r](U)p.m(C) | | p.m(A)p.m(U)p.m(U)p.m(U)p.m(U) | |
| | p.[fl2r](A)p.m(U)p.m(A)p.m(G) | | [ps].m(A)[ps].m(A) | |
| | [ps].m(U)[ps].m(U) | | | |

5vp_una2moe 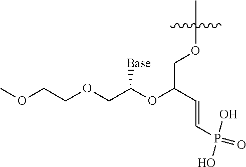
5am6_p_m

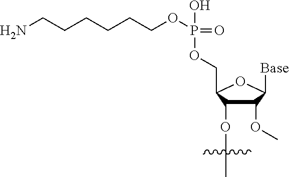

In this table, m indicates the 2′-O-methyl (2′-OMe) ribose modification; [fl2r] indicates the 2′-deoxy-2′-fluoro (2′-F) ribose modification; [ips] indicates phosphorothioate (PS) linkage; p indicates phosphodiester (PO) linkage

Preparation of Anti-TfR1 Antibody—2 Different siRNA Conjugate

The DAR2 AOC with two different siRNAs was prepared by adding 10 molar equivalents (meq) of TCEP to the solution containing antibody (hTfR1.mAb) at 24 g/L in 50 mM citrate and 300 mM sucrose at pH 6.5 and incubating at room temperature for 18 hours. The reaction mixture was diafiltered with a 50 kDa MWCO membrane and 8 diavolumes of 25 mM phosphate buffer, pH 7.5 bringing the antibody concentration to 27.18 g/L. 1.5 meq of ZnCl₂ was added to the reaction mixture and incubated at room temperature for 30 minutes followed by 10 meq of dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 15.08 g/L. A solution of EDTA (500 mM) was added to the antibody solution to reach a final EDTA concentration of 5 mM. After 15 minutes, 1.05 meq of siRNA (siRNA1) with MCC linker attached to the 5′ end of the passenger strand was added to the antibody. The reaction mixture was incubated at room temperature for 30 minutes, followed by analysis by strong anion exchange (SAX) chromatography to quantify the siRNA to antibody ratio (DAR). The reaction mixture was purified by SAX chromatography to isolate DAR1, which was then diafiltered with a 50 kDa MWCO membrane and 8 diavolumes of PBS bringing the antibody concentration to 13.05 g/L. 1.4 EQ of siRNA2 with MCC linker attached to the 5′ end of the passenger strand was added to the isolated DAR1. After 30 minutes, the reaction mixture was analyzed by SAX chromatography and further characterized by intact mass spectrometry.

The results indicate that this method can be used to generate a DAR2 AOC with two different siRNA sequences.

| | Observed | Theoretical | Error |
|---|---|---|---|
| Sample | Mass | Mass | (ppm) |
| anti-TfR1 | 171,871.85 | 171,871.84 | 0 |
| antibody- | | | |
| siRNA DAR2 | | | |

### Example 18: Preparation of DAR 1 and 2 Antibody-siRNA Conjugate with Lipid-siRNA

### Materials and Methods

The human Tfr1 antibody used is described in Example 1.

The sequences of the lipid-siRNAs used in Example 18 are shown in Table 17.

TABLE 17

| | | SEQ | | SEQ |
|---|---|---|---|---|
| | | ID | | ID |
| siRNA | Guide Strand | NO: | Passenger Strand | NO: |
| Lipid- | [5vp_una2moe](U)[ps].[fl2r](A) | 13 | [5am6_p_m](C)[ps].m(C)[ps].[rc22] | 14 |
| siRNA1 | [ps].m(A)p.m(U)p.m(A)p.[fl2r](C) | | (G)p.m(A)p.m(C)p.m(A)p.[fl2r](U)p. | |
| | p.m(C)p.m(G)p.m(A)p.m(G)p.m | | [fl2r](U)p.[fl2r](C)p.m(C)p.m(U)p.m | |
| | (G)p.m(A)p.m(A)p.[fl2r](U)p.m(G) | | (C)p.m(G)p.m(G)p.m(U)p.m(A)p.m | |
| | p.[fl2r](U)p.m(C)p.m(G)p.m(G) | | (U)[ps].m(U)[ps].m(A) | |
| | [ps].m(U)[ps].m(U) | | | |
| Lipid- | [5vp_una2moe](U)[ps].[fl2r](A) | 13 | [5am6_p_m](C)[ps].m(C)[ps].m(G)p. | 15 |
| siRNA2 | [ps].m(A)p.m(U)p.m(A)p.[fl2r](C) | | m(A)p.m(C)p.[rc16](A)p.[fl2r](U)p. | |
| | p.m(C)p.m(G)p.m(A)p.m(G)p.m | | [fl2r](U)p.[fl2r](C)p.m(C)p.m(U)p.m | |
| | (G)p.m(A)p.m(A)p.[fl2r](U)p.m(G) | | (C)p.m(G)p.m(G)p.m(U)p.m(A)p.m | |
| | p.[fl2r](U)p.m(C)p.m(G)p.m(G) | | (U)[ps].m(U)[ps].m(A) | |
| | [ps].m(U)[ps].m(U) | | | |

5vp_una2moe 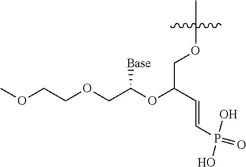
5am6_p_m 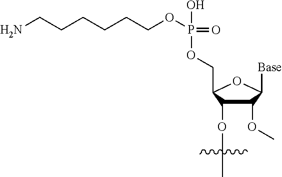
rc16 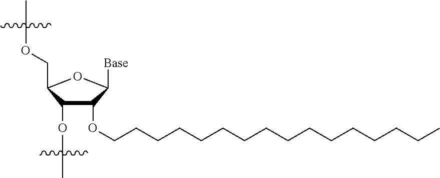
rc22

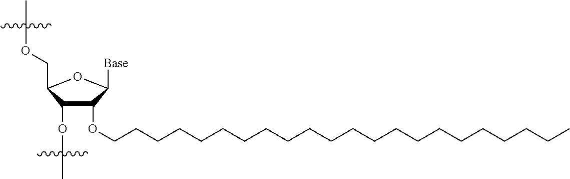

In this table, m indicates the 2′-O-methyl (2′-OMe) ribose modification; [fl2r] indicates the 2′-deoxy-2′-fluoro (2′-F) ribose modification; [ips] indicates phosphorothioate (PS) linkage; p indicates phosphodiester (PO) linkage

### Preparation of Anti-TfR1 Antibody-Lipid-siRNA DAR1 and DAR2 Conjugate

The anti-TfR1 antibody-siRNA conjugate (hTfR1.mAb-MCC-siRNA) was prepared by adding 10 molar equivalents (meq) of TCEP to the solution containing antibody at 24 g/L in 50 mM citrate and 300 mM sucrose at pH 6.5 and incubating at room temperature for 18 hours. The reaction mixture was diafiltered with a 50 kDa MWCO membrane and 8 diavolumes of 25 mM phosphate buffer, pH 7.5 bringing the antibody concentration to 29.80 g/L. 1.5 meq of ZnCl₂ was added to the reaction mixture and incubated at room temperature for 30 minutes followed by 10 meq of dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 10.55 g/L. A solution of EDTA (500 mM) was added to the antibody solution to reach a final EDTA concentration of 5 mM. After 15 minutes, 1.05 or 2.15 meq of siRNA (lipid-siRNA1 or lipid siRNA2) with MCC linker attached to the 5′ end of the passenger strand was added to the antibody. The reaction mixture was incubated at room temperature for 30 minutes, followed by analysis by strong anion exchange (SAX) chromatography to quantify the siRNA to antibody ratio (DAR).

The results indicate that this zinc method can be used to generate AOCs with lipid-siRNAs that are >70% DAR1 or >70% DAR2.

### SAX AUC Results

| | total siRNA- | | | |
|---|---|---|---|---|
| Payload | MCC mEQ | mAb | D1 | D2 |
| Lipid- | 1.05 | 10.5 | 79.1 | 10.3 |
| siRNA1 | 2.15 | 9.2 | 11.9 | 78.9 |
| Lipid- | 1.05 | 10 | 79 | 10.9 |
| siRNA2 | 2.15 | 8.8 | 13.4 | 77.7 |

### Example 19: Preparation of DAR1 Antibody-siRNA Conjugate with Alternate Zinc Salts

### Materials:

The human Tfr1 antibody used is described in Example 1. The siRNA used is described in Example 1.

| | SEQ | | SEQ |
|---|---|---|---|
| | ID | | ID |
| Guide strand | NO: | Passenger strand | NO: |
| m(U)[ps].[fl2r](U)[ps].m(C)p.m(G) | 1 | [5am6_p_m](C)[ps].m(C)[ps].m(C)p. | 2 |
| p.m(A)p.[fl2r](A)p.m(G)p.m(A) | | m(U)p.m(A)p.m(G)p.[fl2r](A)p.[fl2r] | |
| p.m(C)p.m(A)p.m(G)p.m(U)p.m | | (A)p.[fl2r](C)p.m(U)p.m(G)p.m(U)p. | |
| (U)p.[fl2r](C)p.m(U)p.[fl2r](A)p. | | m(C)p.m(U)p.m(U)p.m(C)p.m(G)[ps]. | |
| m(G)p.m(G)p.m(G)[ps].m(U)[ps]. | | m(A)[ps].m(A) | |
| m(U) | | | |

5am6_p_m

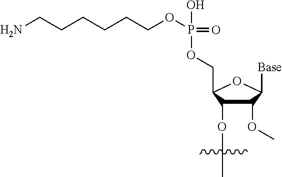

In this table, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage

Preparation of Anti-TfR1 Antibody-siRNA DART with Different Zinc Salts

The anti-TfR1 antibody-siRNA conjugate (hTfR1.mAb-MCC-siRNA) was prepared by adding 10 molar equivalents (meq) of TCEP to the solution containing antibody at 24 g/L in 50 mM citrate and 300 mM sucrose at pH 6.5 and incubating at room temperature for 18 hours. The reaction mixture was diafiltered with a 50 kDa MWCO membrane and 8 diavolumes of 25 mM phosphate buffer, pH 7.5 bringing the antibody concentration to 29.20 g/L. 1.5 meq of Zinc salt (zinc citrate, zinc trifluoromethane sulfonate, or zinc acetylacetonate) was added to the reaction mixture and incubated at room temperature for 30 minutes followed by 10 meq of dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 10.7-10.9 g/L. A solution of EDTA (500 mM) was added to the antibody solution to reach a final EDTA concentration of 5 mM. After 15 minutes, 1.05 meq of siRNA with MCC linker attached to the 5′ end of the passenger strand was added to the antibody. The reaction mixture was incubated at room temperature for 30 minutes, followed by analysis by strong anion exchange (SAX) chromatography to quantify the siRNA to antibody ratio (DAR).

### SAXAUC Results

| Zinc Salt | mAb | D1 | D2 |
|---|---|---|---|
| Zinc Citrate | 12 | 75.2 | 11.7 |
| Zinc Trifluoromethane | 15.9 | 70.2 | 13.9 |
| sulfonate | | | |
| Zinc Acetylacetonate | 14.7 | 71.6 | 13.7 |

### Example 20: Measurement of Antibody-Zinc Intermediate Complex Using SEC-MS

### Materials

The anti-hTfR1 (AV01) and anti-HER2 (Trastuzumab) antibodies are described in Examples 1 and 10.

### Methods

The method for strong anion exchange (SAX) method for analyzing conjugates is described in Example 1.

### Preparation of Antibody-Zinc Intermediate for Analysis by MS

The intermediate was prepared by adding 10 meq of TCEP to the solution containing the anti-TfR1 monoclonal antibody at 20 g/L in 50 mM citrate and 300 mM sucrose at pH 6.5 or anti-HER2 monoclonal antibody at 20 g/L in histidine buffer pH 6, and incubating at room temperature for 2 hours. The reaction mixture was diafiltered with a 50 kDa MWCO membrane and 8 diavolumes of 25 mM phosphate buffer at pH 7.5 bringing the antibody concentration to 10 mg/ml. 1.5 meq of ZnCl₂ was added to the reaction mixture and incubated at room temperature for 30 minutes followed by 10 meq of dehydroascorbic acid incubated at room temperature for 1 hour. The reaction mixture was diafiltered with a 50 kDa MWCO membrane and 8 diavolumes of 25 mM phosphate buffer, pH 7.5 bringing the monoclonal antibody concentration to 10 g/L. The intermediate was stored at −80 C until the next step.

### Deglycosylation and MS

The antibody-zinc complex was deglycosylated with PNGase F and then analyzed by SEC-MS to determine the relative amounts antibody with and without zinc bound, and the number of zinc ions bound per molecule. This experiment was performed with both anti-TfR1 antibody and trastuzumab. Both molecules contained 60-70% antibody-zinc complex with only one zinc ion, and 30-40% unbound antibody. These results suggest that a zinc intermediate is formed and maintained prior to removal by EDTA.

| Sample | Species | % Abundance | Observed Mass |
|---|---|---|---|
| anti-TfR1 | anti-TfR1 antibody | 37 | 144409.74 |
| antibody | anti-TfR1 antibody + Zn⁺ | 58 | 144474.79 |
| | anti-TfR1 antibody + | 4 | 144637.02 |
| | Zn⁺ + glycation | | |
| Trastuzumab | Trastuzumab | 26 | 145165.36 |
| | Trastuzumab + Zn⁺ | 58 | 145233.55 |
| | Trastuzumab + glycation | 2 | 145329.34 |
| | Trastuzumab + | 14 | 145394.59 |
| | Zn⁺ + glycation | | |

### Example 21: Preparation of Anti-TfR1 Antibody-PMO/ASO Conjugates

### Materials

The antibody is described in Example 1. The PMO and ASO used have the sequences below.

TABLE 18

| | | SEQ ID |
|---|---|---|
| | Sequence | NO |
| PMO | [mph](C)[pda].[mph](G)[pda].[mph](C)[pda].[mph](C)[pda].[mph](G) | 333 |
| | [pda].[mph](C)[pda].[mph](C)[pda].[mph](A)[pda].[mph](T)[pda].[mph](T) | |
| | [pda].[mph](T)[pda].[mph](C)[pda].[mph](T)[pda].[mph](C)[pda].[mph](A) | |
| | [pda].[mph](A)[pda].[mph](C)[pda].[mph](A)[pda].[mph](G)[pda].[mph] | |
| | (A)[pda].[mph](T)[pda].[mph](C)[pda].[mph](T)[pda].[mph](G)[pda].[mph] | |
| | (T)[pda].[mph](C)[3bAla] | |
| ASO | [5am6_p_lna]([5mC])[ps].[lna](A)[ps].[moe](G)[ps].[moe]([5mC])[ps].d | 334 |
| | (G)[ps].d(C)[ps].d(C)[ps].d(C)[ps].d(A)[ps].d(C)[ps].d(C)[ps].d(A)[ps]. | |
| | [moe](G)[ps].[moe](T)[ps].[lna]([5mC])[ps].[lna](A) | |

5am6_p_lna

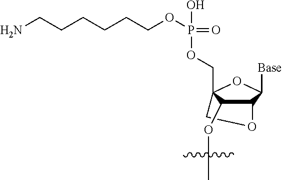

In this table, [mph](N)[pda] refers a phosphodiamidate morpholino nucleotide with base N (base N refers any base, e.g., A, T, G, C, U, or a derivative thereof); [5mC] indicates the 5-methylcytosine; [moe] indicates the 2′-O-methoxyethyl (2′-O-MOE) ribose modification; [ps] indicates phosphorothioate (PS) linkage; p indicates phosphodiester (PO) linkage; [3bAla] indicates a beta alanine at the 3′end.

### Methods

### Hydrophobic Interaction Chromatography (HIC) Method for Analyzing Conjugates

Drug to antibody ratio (DAR) of the antibody-PMO conjugate was determined by HIC using the fluorescence detector with an excitation wavelength of 280 nm and emission of 345 nm. Relative % area of the unreacted mAb (DAR0) and conjugated antibody species (DAR1, DAR2, etc.) was determined. Separation was achieved using a MabPac™ HIC-Butyl column (5 um, 4.6×100 mm) and gradient elution with ammonium sulfate, phosphate buffer and isoproponal mobile phases.
MPA: 1.2 M Ammonium Sulfate, 100 mM Phosphate Buffer pH 7.2
MPB: 100 mM Phosphate Buffer, 20% Isopropanol pH 7.2
FLD: Ex: 280 Em: 345
Column: Thermo Scientific, MabPac™ HIC-Butyl column (5 um, 4.6×100 mm)

$$Flow rate = 1 ml / min$$

### Gradient

| Time (min) | % B |
|---|---|
| 0 | 0 |
| 1.5 | 0 |
| 26.5 | 100 |
| 28.5 | 100 |
| 29 | 0 |
| 33 | 0 |

### SAX Method for Analyzing ASO Conjugates

Drug to antibody ratio (DAR) of the antibody-ASO conjugate was determined by strong anion-exchange chromatography (SAX-HPLC) using the fluorescence detector with an excitation wavelength of 280 nm and emission of 345 nm. Different species in the bioconjugate sample were separated based on their different affinity to the anion exchanger. Relative % area of the unreacted mAb (DAR0) and conjugated antibody species (DAR1, DAR2) was determined. Separation was achieved using a Thermo ProPAC SAX column and gradient elution with tris buffer and sodium chloride mobile phase.
MPA: 10 mM Tris pH 7.2 20% Ethanol
MPB: 10 mM Tris, 1.5M NaCl pH 7.2 20% Ethanol
FLD: Ex: 280 Em: 345
Column: Thermo Scientific, ProPac™ SAX-10, Bio LC™, 4×250 mm

$$Flow rate = 0.9 ml / min$$

### Gradient

| Time (min) | % B |
|---|---|
| 0 | 10 |
| 3 | 10 |
| 13 | 100 |
| 16 | 100 |
| 17 | 10 |
| 22 | 10 |

### Preparation of Anti-TfR1 Antibody-PMO Conjugate

The anti-TfR1 antibody-PMO conjugate (hTfR1.mAb-MCC-PMO) was prepared by adding 10 molar equivalents (meq) of TCEP to the solution containing antibody at 24 g/L in 50 mM citrate and 300 mM sucrose at pH 6.5 and incubating at room temperature for 18 hours. The reaction mixture was diafiltered with a 50 kDa MWCO membrane and 8 diavolumes of 25 mM phosphate buffer, pH 7.5 bringing the antibody concentration to 29.8 mg/ml. 1.5 meq of ZnCl₂ (0.229 mM) was added to the reaction mixture and incubated at room temperature for 30 minutes followed by 10 meq of L-dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 10.55 mg/mL. A solution of EDTA (500 mM) was added to the antibody solution to reach a final EDTA concentration of 5 mM. After 15 minutes, 74.8 uL or 119.2 uL of PMO with MCC linker attached (A260=281) was added to the antibody (8.44 mg). The reaction mixture was incubated at room temperature for 30 minutes, followed by analysis by HIC chromatography to quantify the PMO to antibody ratio (DAR). The results indicate that this zinc method can be applied to oligonucleotides other than siRNAs.

### RP Analysis Results:

| PMO | | | | | |
|---|---|---|---|---|---|
| (uL) | mAb | D1 | D2 | D3 | D4 |
| 74.8 | 15.2 | 17.6 | 60.9 | 6.2 | 0 |
| 119.2 | 9.6 | 6 | 69.8 | 11.3 | 3.2 |

### Preparation of Anti-TfR1 Antibody-ASO Conjugate

The anti-TfR1 antibody-ASO conjugate (hTfR1.mAb-MCC-ASO) was prepared by adding 10 molar equivalents (meq) of TCEP to the solution containing antibody at 24 g/L in 50 mM citrate and 300 mM sucrose at pH 6.5 and incubating at room temperature for 18 hours. The reaction mixture was diafiltered with a 50 kDa MWCO membrane and 8 diavolumes of 25 mM phosphate buffer, pH 7.5 bringing the antibody concentration to 29.8 mg/ml. 1.5 meq of ZnCl₂ (0.229 mM) was added to the reaction mixture and incubated at room temperature for 30 minutes followed by 10 meq of L-dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 10.55 mg/mL. A solution of EDTA (500 mM) was added to the antibody solution to reach a final EDTA concentration of 5 mM. After 15 minutes, the solution (7.38 mg of antibody) was brought to approximately 300 mM NaCl and 177.6 ul of ASO (A260=100.6) with MCC linker attached was added to the antibody. The reaction mixture was incubated at room temperature for 30 minutes, followed by analysis by SAX chromatography to quantify the ASO to antibody ratio (DAR). The results indicate that this zinc method can be applied to oligonucleotides other than siRNAs.

### SAX Analysis Results:

| mAb | D1 | D2 | D3 |
|---|---|---|---|
| 10.7 | 4.2 | 80.5 | 4.5 |

### Example 22: Preparation of Anti-TfR1 Antibody-siRNA Conjugate Using Alternate Buffers for Zinc Incubation with Antibody

### Materials

The antibody and siRNA used are described in Example 1.

| | SEQ | | SEQ |
|---|---|---|---|
| | ID | | ID |
| Guide strand | NO: | Passenger strand | NO: |
| m(U)[ps].[fl2r](U)[ps].m(C)p.m | 1 | [5am6_p_m](C)[ps].m(C)[ps].m(C)p. | 2 |
| (G)p.m(A)p.[fl2r](A)p.m(G)p.m(A) | | m(U)p.m(A)p.m(G)p.[fl2r](A)p.[fl2r] | |
| p.m(C)p.m(A)p.m(G)p.m(U)p.m | | (A)p.[fl2r](C)p.m(U)p.m(G)p.m(U)p. | |
| (U)p.[fl2r](C)p.m(U)p.[fl2r](A)p. | | m(C)p.m(U)p.m(U)p.m(C)p.m(G)[ps]. | |
| m(G)p.m(G)p.m(G)[ps].m(U)[ps]. | | m(A)[ps].m(A) | |
| m(U) | | | |

5am6_p_m

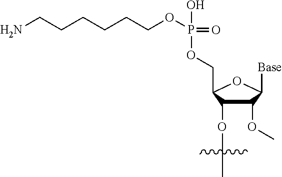

In this table, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage

The anti-TfR1 antibody-siRNA conjugate (hTfR1.mAb-MCC-siRNA) was prepared by adding 10 molar equivalents (meq) of TCEP to the solution containing antibody at 24 g/L in 50 mM citrate and 300 mM sucrose at pH 6.5 and incubating at room temperature for 18 hours. The reaction mixture was diafiltered with a 50 kDa MWCO membrane and 8 diavolumes of either 25 mM phosphate buffer, pH 7.5, 10 mM HEPES pH 7.55, or 10 mM MES pH 7.25. The antibody concentration was brought to 27.64 mg/ml, 28.4 mg/mL or 28.25 mg/mL in 10 mM HEPES, 10 MES or 25 mM phosphate buffer respectively. 1.5 meq of ZnCl₂ (0.229 mM) was added to the reaction mixture and incubated at room temperature for 30 minutes followed by 10 meq of L-dehydroascorbic acid incubated at room temperature for 60 min. The reaction mixture was diafiltered with a 50 kDa MWCO membrane and 8 diavolumes of 25 mM phosphate buffer, pH 7.5 bringing the antibody concentration to 26.99 mg/mL, 29.1 mg/mL, 29.6 mg/mL for reactions that were incubated in zinc in buffers of 10 mM HEPES, 10 mM MES or 25 mM phosphate buffer respectively. A solution of EDTA (500 mM) was added to the antibody solution to reach a final EDTA concentration of 5 mM. After 15 minutes, 1.05 meq of siRNA with MCC linker attached to the 5′ end of the passenger strand was added to the antibody. The reaction mixture was incubated at room temperature for 30 minutes, followed by analysis by HIC chromatography to quantify the siRNA to antibody ratio (DAR). The results indicate that many buffers can be used for the antibody incubation step with zinc.

### SAX AUC Results

| Zinc Incubation Buffer | mAb | D1 | D2 |
|---|---|---|---|
| 10 mM HEPES | 10.4 | 79.6 | 9.9 |
| 10 mM MES | 10.3 | 80.1 | 9.6 |
| 25 mM Phosphate Buffer | 9.7 | 81.6 | 8.8 |

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A method of preparing a binding moiety-oligonucleotide conjugate comprising the steps of:
a) contacting a binding moiety in an acidic buffer with a reducing agent and a transition metal or its salts thereof to generate a first mixture;
b) contacting the first mixture with an oxidizing agent to generate a second mixture;
c) adding an oligonucleotide molecule conjugated with a linker to the second mixture to generate a binding moiety-oligonucleotide conjugate comprising the binding moiety conjugated to the oligonucleotide molecule via the linker; and
d) isolating the binding moiety-oligonucleotide conjugate, thereby preparing the binding moiety-oligonucleotide conjugate.

2. The method of claim 1, wherein the acidic buffer has a pH of less than about 7.0.

3. The method of claim 1, wherein the acidic buffer comprises a citrate/sucrose buffer or a histidine buffer.

4. The method of claim 1, wherein the step a) comprises about 10 molar equivalents of the reducing agent.

5. The method of claim 1, wherein the reducing agent comprises tris(2-carboxyethyl)phosphine (TCEP) or dithiothreitol (DTT).

6. The method of claim 1, wherein the step a) comprises about 1-10 molar equivalents of the transition metal or its salts thereof.

7. The method of claim 1, wherein the transition metal or its salts thereof comprises zinc (Zn) or its salts thereof, silver (Ag) or its salts thereof, or gold (Au) or its salts thereof.

8. The method of claim 1, wherein (i) the binding moiety contacts the reducing agent and the transition metal or its salts thereof concurrently, or (ii) the binding moiety contacts the reducing agent prior to contacting the transition metal or its salts thereof.

9. The method of claim 1, wherein the step b) comprises less than about 15, 14, 13, 12, or 11 molar equivalents of the oxidizing agent.

10. The method of claim 1, wherein the step b) comprises about 1-10 molar equivalents of the oxidizing agent.

11. The method of claim 1, wherein the oxidizing agent comprises dehydroascorbic acid (DHAA).

12. The method of claim 1, wherein the step c) comprises about 1-3 molar equivalents of the oligonucleotide molecule conjugated with the linker (oligonucleotide-linker).

13. The method of claim 1, wherein the step d) comprises isolating the binding moiety-oligonucleotide conjugate via chromatography or filtration.

14. The method of claim 13, wherein the chromatography comprises strong anion chromatography (SAX).

15. The method of claim 13, wherein the filtration comprises tangential flow filtration (TFF).

16. The method of claim 1, wherein the method further comprises e) adding an additional oligonucleotide-linker to the isolated binding moiety-oligonucleotide conjugate, wherein the additional oligonucleotide-linker comprises an oligonucleotide molecule that is different from the oligonucleotide molecule in step c), thereby obtaining the binding moiety-oligonucleotide conjugate having different oligonucleotides.

17. The method of claim 1, wherein a yield of the isolated binding moiety-oligonucleotide conjugate having the DAR of about 1 or about 2 is greater than 50%, 60%, 70%, 80% or more.

18. A method of preparing an antibody-oligonucleotide conjugate or an antigen binding fragment-oligonucleotide conjugate comprising steps of:
a) contacting an antibody or antigen binding fragment thereof with a reducing agent;
b) reacting the antibody or antigen binding fragment thereof with a transition metal or its salts thereof;
c) contacting the antibody or antigen binding fragment thereof from step b) with an oxidizing agent;
d) quenching the oxidizing agent using a mineral or organic buffer; and
e) chelating the transition metal using a chelating agent; and
f) contacting an oligonucleotide molecule conjugated with a linker (oligonucleotide-linker) to the antibody or antigen binding fragment thereof, thereby generating an antibody-oligonucleotide conjugate or antigen binding fragment-oligonucleotide conjugate.

19. The method of claim 18, wherein the organic or mineral buffer comprises a borate buffer.

20. The method of claim 18, wherein the method further comprises purifying the antibody or antigen binding fragment thereof obtained from the step (a).

21. The method of claim 18, wherein the method further comprises capping the antibody or antigen binding fragment thereof in the antibody-oligonucleotide conjugate or the antigen binding fragment-oligonucleotide conjugate generated from the step f) using an alkylating reagent.

22. The method of claim 18, wherein the alkylating agent comprises N-ethylmaleimide (NEM).

23. The method of claim 18, wherein the step g) comprises about 1-1.5 molar equivalents of the alkylating agent.

24. The method of claim 18, wherein the chelating agent comprises ethylenediaminetetraacetic acid (EDTA).

25. The method of claim 18, wherein the method further comprises isolating the antibody-oligonucleotide conjugate or antigen binding fragment-oligonucleotide conjugate via chromatography or filtration.


SEQUENCE LISTING (334 sequences)

SEQ ID NO: 1 - RNA, 21 nt, synthetic construct
  modified_base 19..21: mod_base = OTHER; note = phosphorothioate internucleotide linkage
  source 1..21: mol_type = other RNA
  modified_base 6: mod_base = OTHER; note = 2'-fluoro ribonucleotide
  modified_base 17..21: mod_base = OTHER; note = 2'-O-methyl ribonucleotide
  modified_base 14: mod_base = OTHER; note = 2'-fluoro ribonucleotide
  modified_base 1: mod_base = OTHER; note = 2'-O-methyl ribonucleotide
  modified_base 16: mod_base = OTHER; note = 2'-fluoro ribonucleotide
  modified_base 3..5: mod_base = OTHER; note = 2'-O-methyl ribonucleotide
  modified_base 1..3: mod_base = OTHER; note = phosphorothioate internucleotide linkage
  modified_base 2: mod_base = OTHER; note = 2'-fluoro ribonucleotide
  modified_base 15: mod_base = OTHER; note = 2'-O-methyl ribonucleotide
  modified_base 7..13: mod_base = OTHER; note = 2'-O-methyl ribonucleotide
ttcgaagacagttctagggtt

SEQ ID NO: 2 - RNA, 19 nt, synthetic construct
  modified_base 2..6: mod_base = OTHER; note = 2'-O-methyl ribonucleotide
  modified_base 17..19: mod_base = OTHER; note = phosphorothioate internucleotide linkage
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 7..9: mod_base = OTHER; note = 2'-fluoro ribonucleotide
  modified_base 1..3: mod_base = OTHER; note = phosphorothioate internucleotide linkage
  modified_base 10..19: mod_base = OTHER; note = 2'-O-methyl ribonucleotide
  source 1..19: mol_type = other RNA
ccctagaactgtcttcgaa

SEQ ID NO: 3 - RNA, 21 nt, synthetic construct
  modified_base 16: mod_base = OTHER; note = 2'-fluoro ribonucleotide
  modified_base 6: mod_base = OTHER; note = 2'-fluoro ribonucleotide
  modified_base 19..21: mod_base = OTHER; note = phosphorothioate internucleotide linkage
  modified_base 17..21: mod_base = OTHER; note = 2'-O-methyl ribonucleotide
  modified_base 7..13: mod_base = OTHER; note = 2'-O-methyl ribonucleotide
  modified_base 14: mod_base = OTHER; note = 2'-fluoro ribonucleotide
  source 1..21: mol_type = other RNA
  modified_base 15: mod_base = OTHER; note = 2'-O-methyl ribonucleotide
  modified_base 3..5: mod_base = OTHER; note = 2'-O-methyl ribonucleotide
  modified_base 2: mod_base = OTHER; note = 2'-fluoro ribonucleotide
  modified_base 1..3: mod_base = OTHER; note = phosphorothioate internucleotide linkage
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
ttcgaagacagttctagggtt

SEQ ID NO: 4 - RNA, 19 nt, synthetic construct
  modified_base 17..19: mod_base = OTHER; note = phosphorothioate internucleotide linkage
  modified_base 10..19: mod_base = OTHER; note = 2'-O-methyl modified nucleotide
  modified_base 4..6: mod_base = OTHER; note = 2'-O-methyl ribonucleotide
  modified_base 2: mod_base = OTHER; note = 2'-O-methyl ribonucleotide
  modified_base 3: mod_base = OTHER; note = (2R,3S,4R,5R)-5-(hydroxymethyl)-3-(icosyloxy)tetrah ydrofuran-2,4-diol nucleotide
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 7..9: mod_base = OTHER; note = 2'-fluoro ribonucleotide
  modified_base 1..3: mod_base = OTHER; note = phosphorothioate internucleotide linkage
ccctagaactgtcttcgaa

SEQ ID NO: 5 - RNA, 19 nt, synthetic construct
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 4..6: mod_base = OTHER; note = 2'-O-methyl ribonucleotide
  modified_base 17..19: mod_base = OTHER; note = phosphorothioate internucleotide linkage
  source 1..19: mol_type = other RNA
  modified_base 1..3: mod_base = OTHER; note = phosphorothioate internucleotide linkage
  modified_base 7..9: mod_base = OTHER; note = 2'-fluoro ribonucleotide
  modified_base 10..19: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 2: mod_base = OTHER; note = 2'-O-methyl ribonucleotide
ccctagaactgtcttcgaa

SEQ ID NO: 6-10 - skipped (5 entries)

SEQ ID NO: 11 - RNA, 21 nt, synthetic construct
  modified_base 7..13: mod_base = OTHER; note = 2'-O-methyl ribonucleotide
  modified_base 1..3: mod_base = OTHER; note = phosphorothioate internucleotide linkage
  modified_base 6: mod_base = OTHER; note = 2'-fluoro ribonucleotide
  modified_base 15: mod_base = OTHER; note = 2'-O-methyl ribonucleotide
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 16: mod_base = OTHER; note = 2'-fluoro ribonucleotide
  modified_base 2: mod_base = OTHER; note = 2'-fluoro ribonucleotide
  modified_base 3..5: mod_base = OTHER; note = 2'-O-methyl ribonucleotide
  source 1..21: mol_type = other RNA
  modified_base 14: mod_base = OTHER; note = 2'-fluoro ribonucleotide
  modified_base 17..21: mod_base = OTHER; note = 2'-O-methyl modified nucleotide
  modified_base 17..19: mod_base = OTHER; note = phosphorothioate internucleotide linkage
ttaaaatctacagtcatagtt

SEQ ID NO: 12 - RNA, 19 nt, synthetic construct
  source 1..19: mol_type = other RNA
  modified_base 7..9: mod_base = OTHER; note = 2'-fluoro ribonucleotide
  modified_base 17..19: mod_base = OTHER; note = phosphorothioate internucleotide linkage
  modified_base 1..3: mod_base = OTHER; note = phosphorothioate internucleotide linkage
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 10..19: mod_base = OTHER; note = 2'-O-methyl modified nucleotide
  modified_base 2..6: mod_base = OTHER; note = 2'-O-methyl modified nucleotide
ctatgactgtagattttaa

SEQ ID NO: 13 - RNA, 21 nt, synthetic construct
  modified_base 1..3: mod_base = OTHER; note = phosphorothioate internucleotide linkage
  modified_base 2: mod_base = OTHER; note = 2'-fluoro ribonucleotide
  modified_base 14: mod_base = OTHER; note = 2'-fluoro ribonucleotide
  modified_base 3..5: mod_base = OTHER; note = 2'-O-methyl modified nucleotide
  modified_base 7..13: mod_base = OTHER; note = 2'-O-methyl modified nucleotide
  modified_base 16: mod_base = OTHER; note = 2'-fluoro ribonucleotide
  modified_base 17..21: mod_base = OTHER; note = 2'-O-methyl modified nucleotide
  source 1..21: mol_type = other RNA
  modified_base 6: mod_base = OTHER; note = 2'-fluoro ribonucleotide
  modified_base 15: mod_base = OTHER; note = 2'-O-methyl modified nucleotide
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 19..21: mod_base = OTHER; note = phosphorothioate internucleotide linkage
taataccgaggaatgtcggtt

SEQ ID NO: 14 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 17..19: mod_base = OTHER; note = phosphorothioate internucleotide linkage
  modified_base 2: mod_base = OTHER; note = 2'-O-methyl modified nucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 7..9: mod_base = OTHER; note = 2'-fluoro ribonucleotide
  modified_base 10..19: mod_base = OTHER; note = 2'-O-methyl modified nucleotide
  modified_base 4..6: mod_base = OTHER; note = 2'-O-methyl modified nucleotide
  modified_base 1..3: mod_base = OTHER; note = phosphorothioate internucleotide linkage
ccgacattcctcggtatta

SEQ ID NO: 15 - RNA, 19 nt, synthetic construct
  modified_base 7..9: mod_base = OTHER; note = 2'-fluoro ribonucleotide
  modified_base 17..19: mod_base = OTHER; note = phosphorothioate internucleotide linkage
  modified_base 10..19: mod_base = OTHER; note = 2'-O-methyl modified nucleotide
  source 1..19: mol_type = other RNA
  modified_base 6: mod_base = OTHER; note = (2R,3S,4R,5R)-3-(hexadecyloxy)-5-(hydroxymethyl)tet rahydrofuran-2,4-diol nucleotide
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 1..3: mod_base = OTHER; note = phosphorothioate internucleotide linkage
  modified_base 2..5: mod_base = OTHER; note = 2'-O-methyl modified nucleotide
ccgacattcctcggtatta

SEQ ID NO: 16-222 - skipped (207 entries)

SEQ ID NO: 223 - protein, 4 aa, synthetic construct
GGFG

SEQ ID NO: 224 - protein, 4 aa, synthetic construct
ALAL

SEQ ID NO: 225 - protein, 4 aa, synthetic construct
GFLG

SEQ ID NO: 226 - protein, 17 aa, synthetic construct
  VARIANT 13: note = Q or E
  VARIANT 6: note = N or Q
EINPIXGRSNYAXKFQG

SEQ ID NO: 227 - protein, 11 aa, synthetic construct
  VARIANT 8: note = N or S
RTSENIYXNLA

SEQ ID NO: 228 - protein, 7 aa, synthetic construct
  VARIANT 2: note = A or G
  VARIANT 7: note = D or E
AXTNLAX

SEQ ID NO: 229 - protein, 10 aa, synthetic construct
  VARIANT 10: note = May be deleted
QHFWGTPLTF

SEQ ID NO: 230 - protein, 7 aa, synthetic construct
  VARIANT 7: note = D or E
AATNLAX

SEQ ID NO: 231-280 - skipped (50 entries)

SEQ ID NO: 281 - protein, 9 aa, synthetic construct
YTFTNYWMH

SEQ ID NO: 282 - protein, 17 aa, synthetic construct
EINPINGRSNYAQKFQG

SEQ ID NO: 283 - protein, 7 aa, synthetic construct
GTRAMHY

SEQ ID NO: 284 - protein, 17 aa, synthetic construct
EINPINGRSNYAEKFQG

SEQ ID NO: 285 - protein, 17 aa, synthetic construct
EINPIQGRSNYAEKFQG

SEQ ID NO: 286 - protein, 11 aa, synthetic construct
RTSENIYNNLA

SEQ ID NO: 287 - protein, 7 aa, synthetic construct
AATNLAD

SEQ ID NO: 288 - protein, 9 aa, synthetic construct
QHFWGTPLT

SEQ ID NO: 289 - protein, 7 aa, synthetic construct
AATNLAE

SEQ ID NO: 290 - protein, 10 aa, synthetic construct
QHFWGTPLTF

SEQ ID NO: 291 - protein, 11 aa, synthetic construct
RTSENIYSNLA

SEQ ID NO: 292 - protein, 7 aa, synthetic construct
AGTNLAD

SEQ ID NO: 293 - protein, 116 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 294 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 295 - protein, 116 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 296 - protein, 116 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 297 - protein, 116 aa, synthetic construct
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 298 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 299 - protein, 107 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 300 - protein, 107 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 301 - protein, 107 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

SEQ ID NO: 302 - protein, 107 aa, synthetic construct
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 303 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 304 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 305 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 306 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 307 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 308 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 310 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 311 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 312 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 313 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 314 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 315 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 316 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 317 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 318 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 319 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 320 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 322 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 324 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 326 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 327 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 214 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 214 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 214 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 6 aa, Homo sapiens
KSCDKT

SEQ ID NO: 332 - protein, 6 aa, Homo sapiens
HTCPPC

SEQ ID NO: 333 - DNA, 26 bp, synthetic construct
  modified_base 1..25: mod_base = OTHER; note = phosphodiamidate morpholino nucleotide
  modified_base 26: mod_base = OTHER; note = morpholino cytosine linked to beta alanine
  source 1..26: mol_type = other DNA
cgccgccatttctcaacagatctgtc

SEQ ID NO: 334 - DNA, 16 bp, synthetic construct
  source 1..16: mol_type = other DNA
  modified_base 14: mod_base = OTHER; note = 2'-O-methoxyethyl thymine
  modified_base 2: mod_base = OTHER; note = locked nucleic acid
  misc_feature 1..4: note = RNA
  misc_feature 5..12: note = DNA
  modified_base 13: mod_base = OTHER; note = 2'-O-methoxyethyl ribonucleotide
  misc_feature 13..16: note = RNA
  modified_base 15: mod_base = OTHER; note = Locked 5-methylcytosine
  modified_base 3: mod_base = OTHER; note = 2'-O-methoxyethyl ribonucleotide
  modified_base 16: mod_base = OTHER; note = locked nucleic acid
  modified_base 1..16: mod_base = OTHER; note = phosphorothioate linkage
  modified_base 4: mod_base = OTHER; note = 2'-O-methoxyethyl 5-methylcytosine
  modified_base 1: mod_base = OTHER; note = 5'aminohexyl phosphate locked 5-methylcytosine
cagcgcccaccagtca